(19) United States
(12) Patent Application Publication
(10) Pub. No.: US 2025/0145693 A1
(43) Pub. Date: May 8, 2025

(54) HERV-K ANTIBODY, CELL, VACCINE, AND DRUG THERAPEUTICS

(71) Applicant: SunnyBay BioTech, Inc., FREMONT, CA (US)

(72) Inventors: Feng WANG-JOHANNING, FREMONT, CA (US); Gary JOHANNING, FREMONT, CA (US)

(21) Appl. No.: 18/692,760

(22) PCT Filed: Sep. 17, 2022
(86) PCT No.: PCT/US2022/076625

§ 371 Date: Mar. 15, 2024

Related U.S. Application Data
(63) Continuation-in-part of application No. PCT/US2021/071505, filed on Sep. 18, 2021.
Application No. PCT/US2021/071505 is a continuation-in-part of application No. PCT/US2021/071506, filed on Sep. 18, 2021.
(60) Provisional application No. 63/352,967, filed on Jun. 16, 2022.

(51) Int. Cl. C07K 16/10; A61K 39/00; A61K 39/42; A61P 35/00; C07K 14/005; C07K 14/725; G01N 33/574

(52) U.S. Cl. CPC C07K 16/1036; A61K 39/0011; A61K 39/42; A61P 35/00; C07K 14/005; C07K 14/7051; G01N 33/57415; A61K 2039/505; A61K 2039/5158; A61K 2039/585; C07K 2317/24; C07K 2317/622; C07K 2317/77; C07K 2317/92; C12N 2740/12022

(57) ABSTRACT

The invention relates to peptides, proteins, nucleic acids, and cells for use in immunotherapeutic methods. In particular, the invention relates to the immunotherapy of cancer. The invention provides T cell receptors (TCRs), tumor infiltrating lymphocytes (TILs), and vaccines that recognize HERV-K. The invention provides TCR sequences generated from tumor infiltrating lymphocytes that recognize HERV-K antigens as peptides bound to the Major Histocompatibility Complex (MHC), resulting in an interaction between the HLA-peptide complex and the CDS TCR. Peptides bound to molecules of the MHC, or peptides as such, can also be targets of antibodies, soluble TCRs, and other binding molecules.

### FIELD OF THE INVENTION

This invention relates generally to cancer antigens.

### BACKGROUND OF THE INVENTION

Human endogenous retroviruses (HERVs) are well-known as genomic repeat sequences, with many copies in the genome. Approximately 8% of the human genome is of retroviral origin. See, Lander et al. Nature. 409, 860-921 (2001). Retroviruses typically lose infectivity because of the accumulation of genetic mutations. These genes are predominantly silent and not expressed in normal adult human tissues, except during pathologic conditions such as cancer.

The most biologically active HERVs are members of the HERV-K family. HERV-K has a complete sequence capable of expressing all the elements needed for a replication-competent retrovirus but remain silent in normal cells. Larsson, Kato, & Cohen, Current Topics Microbiol. Immunol., 148, 115-132 (1989); Ono, Yasunaga, Miyata, & Ushikubo, J. Virol. 60, 589-598 (1986). The inventors and others have reported that, sometimes, such as in tumors, expression of HERV-K is activated, and its envelope protein can be detected in several types of tumors at much higher levels than in normal tissues. See International Pat. Publ. WO 2010/138803 (Board of Regents, the University of Texas System); Wang-Johanning et al., Cancer Res., 77, Abstract nr LB-221 (2017), Johanning et al., Expression of human endogenous retrovirus-K is strongly associated with the basal-like breast cancer phenotype. Sci. Rep., 7, 41960 (2017); and Li et al., Clinical Cancer Research (2017). This association indicates that HERV-K could be an excellent tumor associated antigen and an ideal target for cancer immunotherapy. HERV-K is expressed in tumors and is absent in normal tissues, which minimizes off-target effects.

An important consideration in developing a cancer therapeutic is the expression profile of the tumor associated antigen. HERV-K is transcriptionally active in cancer tissues and cell lines. The inventors specifically identified HERV proteins and sequences in cancer cell lines and patient tumors. The inventors observed the expression of HERVs, especially HERV-K sequences, in breast, lung, prostate, ovarian, colon, pancreatic, and other solid tumors. They also found that the expression of HERV-K env transcripts in breast cancer was specifically associated with basal breast cancer, an aggressive subtype. Johanning et al., Expression of human endogenous retrovirus-K is strongly associated with the basal-like breast cancer phenotype. Sci. Rep., 7, 41960 (2017).

Several diagnostic products can be used as companion diagnostics for patient selection. One strategy targets endogenous viral antigens found only on cancer cells—not on normal tissues. The inventors' group discovered that both HERV-K RNAs (env or gag) and anti-HERV-K antibodies appear in the circulation of cancer patients.

An improved understanding of the tumor microenvironment of breast cancer is important for the design of rational and efficient therapy. One problem that has limited the success of therapy against solid tumors is the absence of tumor antigens highly expressed in tumor cells but not normal cells.

In the inventors' previous work, they showed that the HERV-K Env protein is commonly expressed on the surface of breast cancer cells. Wang-Johanning et al., J. Natl. Cancer Inst., 104, 189-210 (2012). Epithelial-mesenchymal transition (EMT) lowers infiltration of CD4 or CD8 T cells in some tumors. Chae et al., Science Reports, 8, 2918 (2018). HERV-K expression was demonstrated to induce epithelial-mesenchymal transition, leading to an increase in cell motility, both of which favor tumor dissemination. See Lemaitre et al., PLOS Pathog., 13, e1006451 (2017). Overexpression of HERV-K leads to cancer onset and contributes to cancer progression.

### SUMMARY OF THE INVENTION

The inventors found that checkpoint molecule levels in serum and tumor-infiltrating lymphocytes (TILs) are highly correlated to HERV-K antibody titers, especially in aggressive breast cancer patients (patients with invasive ductal carcinoma (IDC) or invasive mammary carcinoma (IMC)). The phenotypic and functional characteristics of TILs in breast cancer are related to HERV-K status, and the combination of checkpoint inhibition and HERV-K antibody therapy could result in better killing efficacy.

HERV-K could be an excellent tumor associated antigen. HERV-K could also be an ideal target for cancer immunotherapy because the virus is absent in normal tissues and expressed in tumors, which minimizes off-target effects.

In a first embodiment, the invention provides therapeutic humanized anti-HERV-K antibodies The invention also provides a fusion therapeutic humanized anti-HERV-K antibody of a bispecific T cell engager (BiTE) for CD3 or CD8, a DNA-encoded BITE (DBITE), or an antibody-drug conjugate (ADC). Cancer cells overexpressing HERV-K can be good targets and good models for the anti-HERV-K humanized antibodies and antibody-drug conjugates of the invention, because more antibodies may be bound per cell.

In a second embodiment, the invention provides two humanized antibody clones (HUM1) generated from bacteria and a humanized antibody generated from mammalian cells (hu6H5). Both clones can bind antigens produced from recombinant HERV-K Env surface fusion protein (KSU) and lysates from MDA-MB-231 breast cancer cells. The hu6H5 generated from mammalian cells was compared with our other forms of anti-HERV-K antibodies. The hu6H5 has binding affinity to HERV-K antigen that is similar to murine antibodies (m6H5), chimeric antibodies (cAb), or humanized antibody (HUM1). The hu6H5 antibody induces cancer cells to undergo apoptosis, inhibits cancer cell proliferation, and kills cancer cells that express HERV-K antigen. Importantly, the hu6H5 antibody was demonstrated to reduce tumor viability in mouse MDA-MB-231 xenografts, and notably was able to reduce cancer cell metastasis to lung and lymph nodes. Mice bearing human breast cancer tumors that were treated with these humanized antibodies prolonged survival compared to control mice that did not receive antibody treatment.

In a third embodiment, the invention provides HERV-K env gene generated from a breast cancer patient as an oncogene which can induce cancer cell proliferation, tumor growth, and metastasis to lungs and lymph nodes. Cells expressing HERV-K showed reduced expression of genes associated with tumor suppression, including Caspases 3 and 9, pRB, SIRT-1 and CIDEA, and increased expression of genes associated tumor formation, including Ras, p-ERK, P-P-38, and beta Catenin.

In a fourth embodiment, the invention provides BiTEs directed against T cell CD3 or CD8 and the tumor-associated antigen HERV-K. The inventors produced such a BiTE, which was comprised of antibodies targeting either CD3 or CD8 and HERV-K (VL-VH 6H5scFv - - - VH-VLhuCD3 or CD8+c-myc+FLAG) or (VL-VH hu6H5scFv - - - VH-VLhuCD3 or huCD8+c-myc+FLAG). FLAG-tag, a peptide recognized by an antibody (DYKDDDDK) (SEQ ID NO: 33) and Myc-tag, a short peptide recognized by an antibody (EQKLISEEDL) (SEQ ID NO: 34).

In a fifth embodiment, the invention provides T cells expressing a lentiviral CAR expression vector that bears a humanized or fully human HERV-K scFv. These T-cells effectively lyse and kill tumor cells from several different cancers. Humanized K-CARs expressed from lentiviral vectors are pan-cancer CAR-Ts.

In a sixth embodiment, the invention provides humanized single chain variable fragment (scFv) antibody. This antibody can bind antigens produced from recombinant HERV-K Env surface fusion protein (KSU) and lysates from MDA-MB-231 breast cancer cells. A CAR produced from this humanized scFv can be cloned into a lentiviral vector. This recombinant vector can be used in combination with therapies, including but are not limited to K-CAR T cells plus checkpoint inhibitors, proinflammatory cytokines such as interleukin (IL)-12 and IL-18, oncolytic viruses, and kinase inhibitors. The kinase inhibitors include but not limited to p-RSK and p-ERK.

In a seventh embodiment, the invention provides HERV-K staining that overlaps in many cases with staining of the serum tumor marker CK. HERV-K can be a CTC marker as well as a target for HERV-K antibody therapy.

In an eighth embodiment, the invention provides HERV-K as a stem cell marker. Targeting of HERV-K can block tumor progression by slowing or preventing growth of cancer stem cells. Targeting of HERV-K with circulating therapeutic antibodies or other therapies can also kill CTCs and prevent metastasis of these circulating cells to distant sites.

In a ninth embodiment, the invention provides that forced overexpression of HERV-K with agents that induce expression of HERV-K by innate immune response (such as Poly I:C treatment) or LTR hypomethylation (such as by 5-Aza) provokes cancer cells to increase production of a target that would make them more susceptible to targeted therapy to include targeted immunotherapy.

In a tenth embodiment, the invention improves an in vivo enrichment technique (IVE: ≈20-fold enhancement) in SCID/beige mice, allowing for rapid expansion and B cell activation. This improved technique can produce many antigen-specific plasmablasts. For donors who have cancer with a higher titer of antibodies, the improved technique uses a protocol with humanized mice (HM) or human tumor mice (HTM) instead SCID/beige mice. For normal donors who do not have cancer and who have no memory B cells, the improved technique uses a protocol with modifications: Mice are treated with cytokine cocktails (days 1, 7, and 14) and boosted by antigens on days 14 and 21. Sera are collected from mice and binding affinity is tested by ELISA. After increased antibody titers are detected, spleens are harvested, analyzed, and used to make hybridomas. Higher antibody titers were detected in mice using an IVE protocol.

In an eleventh embodiment, the invention provides a method to determine cells that not only produce antibodies but are also able to bind antigen and kill cancer cells. This method can efficiently stimulate and expand CD40-B cells to large numbers in high purity (>90%) and induce secretion of their antibodies.

In a twelfth embodiment, the invention provides a method of post-incubation of treated B cells. Glass cover slips are washed and tagged with fluorescent anti-human IgG antibody and read using a microengraving technology to reveal discrete spots that correspond to secretion of antigen-specific antibodies by single B cells.

In a thirteenth embodiment, the invention provides for the development of a platform to determine the binding kinetics and cell-to-cell interactions of every cell in a microwell slab.

In a fourteenth embodiment, the invention strikingly provides significantly enhanced expression of six circulating immune checkpoint proteins in the plasma of breast cancer patients. The invention also provides a marked drop in immune checkpoint protein levels in patients at 6 months or 18 months post-surgery vs. pre-surgery. Importantly, a positive association between soluble immune checkpoint protein molecule levels and HERV-K antibody titers induced by HERV-K expression in the tumor results. HERV-K antibody titers can influence immune checkpoint protein levels in breast cancer. Thus, the expression of HERV-K can control the immune responses of breast cancer patients.

In another aspect, these findings collectively show that the immunosuppressive domain (ISD) of HERV-K is a yet unrecognized immune checkpoint on cancer cells, analogous to the PD-L1 immune checkpoint.

In a fifteenth embodiment, the invention provides that blockade of the ISD with immune checkpoint inhibitors of HERV-K, including but not limited to monoclonal antibodies and drugs targeting the ISD of HERV-K, is a cancer immunomodulator therapy that will allow T cells to continue working and unleash immune responses against cancer as well as enhance existing responses, to promote elimination of cancer cells.

In a sixteenth embodiment, the invention provides humanized and fully human (hTab) antibodies targeting HERV-K. These antibodies enhance checkpoint blockade antibody treatment efficacy. Effective combined cancer therapies include but are not limited to combinations of (a) HERV-K humanized or hTAb (1.5 mg/kg), (b) K-CAR, (c) K-BITE, (d) HERV-K shRNAs or CRISPR/Cas9 genome editing technology to knock down HERV-K gene expression, (e) or preventative or therapeutic HERV-K vaccines, including full-length and truncated HERV-K Env proteins and HERV-K Env peptides. Effective combined cancer therapies include full-length and truncated HERV-K Env proteins and HERV-K Env peptides, combined with factors including but not limited to (a) anti-ICP antibody, (b) cancer chemotherapy, (c) 5-Azacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion, (f) induction of S or G2 phase cell cycle arrest, (g) inhibitors of PI3K/AKT/mTOR or MAPK/ERK signaling pathways, or (f) signal transduction to HIF1α.

In a seventeenth embodiment, the invention provides humanized antibodies targeting HERV-K that can be used for ADCs to deliver the drugs into cancer cells and tumors.

In an eighteenth embodiment, the invention provides antibodies targeting HERV-K that can be used for tumor imaging.

In a nineteenth embodiment, the invention provides a new CAR using hu6H5 scFv.

In a twentieth embodiment, the invention provides a new BITE using hu6H5 scFv including CD3 BiTEs and CD8 BiTEs.

The inventors found HERV-K viral particles present in cancer patient blood. Viral particles were also detected in an invasive ductal carcinoma patient's serum by transmission electron microscopy (TEM) using uranyl acetate (UA) negative staining. In addition, viral particles were found on the inside of MDA-MB-231 xenograft and a metastatic adenocarcinoma (Acc 65) xenograft in mice by transmission electron microscopy. Reverse transcriptase activity was compared in various cells. Pooled plasma fractions obtained from a patient with metastatic adenocarcinoma (Acc 65). MMTV-RT was used as a positive control. The highest RT activity was demonstrated in patient Acc 65.

Full length genes with open reading frames of gag, pol, and env were demonstrated by RT-PCR followed by sequence analysis. The inventors found that full length ENV and SU protein bands were detected in some fractions of an invasive ductal carcinoma (IDC) patient sera, but not in normal female controls using an immunoblot assay with anti-HERV-K monoclonal antibody (m6H5) for detection. Enhanced reverse transcriptase (RT) activities were also demonstrated in cancer patients (Acc 65) and cancer cells, relative to activities in benign or normal female donors without cancer.

The inventors found that overexpression of the HERV-K env gene promotes expression of multiple oncogenes including Ras (especially KRas), p-ERK, c-myc, HIF-1alpha, and others. HERV-K env gene downregulated expression of caspases 3 and 9, p-RB, CIDEA, p-P38, and eNOS. HERV-K env gene downregulated AMPK alpha expression, but upregulated AMPK beta expression. Flow cytometry was used to determine changes in gene expression in MDA-MB-231 cells stably transfected with HERV-K env gene. Down regulated expression of caspases 3 and 9, pRB, SIRT-1, eNOS, AMPK alpha, and CIDEA was demonstrated in 231K compared with 231C cells. 231K cells are MDA-MB-231 cells transduced with an HERV-K expression vector, while 231C cells are MDA-MB-231 cells transduced with an empty control expression vector. Upregulated expression of ERK1, beta catenin, p-p-38, and AMPK beta paralleled up-regulated expression of HERV-K in 231K cells.

The inventors found that the HERV-K env gene isolated from a viral particle was further demonstrated to promote tumor development, especially metastasis, in vitro and in vivo. Mice were inoculated with 231C and 231 K cells, and mouse survival rates were compared. A shorter survival rate was observed in mice inoculated with 231K cells compared with their control cells (231C). The 231K cells metastasized to lungs, lymph nodes, and ascites fluid, and tumor cells cultured from lung and ascites fluid continued to grow.

The inventors found that checkpoint molecule levels in serum and TILs are highly correlated to HERV-K antibody titers, especially in aggressive breast cancer patients (patients with invasive ductal carcinoma or invasive mammary carcinoma (IMC)). The phenotypic and functional characteristics of TILs in breast cancer are related to HERV-K status, and the combination of checkpoint inhibition and HERV-K therapies that include antibodies, T cell receptors (TCRs), vaccines, peptides, shRNAs, and other drugs could result in better killing efficacy.

The invention relates to peptides, proteins, nucleic acids, and cells for use in immunotherapeutic methods. In particular, the invention relates to the immunotherapy of cancer. The invention provides TCRs, TILs, and vaccines that recognize HERV-K. In a twenty-first embodiment, the invention provides TCR sequences generated from TILs that recognize HERV-K antigens as peptides bound to the Major Histocompatibility Complex (MHC), resulting in an interaction between the HLA-peptide complex and the CD8 TCR.

The invention provides viral particles and the oncogene of Kenv isolated from the viral particles.

The invention also relates to tumor-associated HERV-K T cell peptide epitopes, alone or in combination with other tumor-associated HERV-K peptides and proteins that can for example serve as active pharmaceutical ingredients of vaccine compositions that stimulate anti-tumor immune responses, including innate and adoptive immune responses, or to stimulate T cells ex vivo and transfer into patients. Peptides bound to molecules of the Major Histocompatibility Complex, or peptides as such, can also be targets of antibodies, soluble TCRs, and other binding molecules. In a twenty-second embodiment, the invention provides a method for increasing T cell effector function by providing a TCR having the property of recognizing tumor-specific HERV-K protein fragment/Major Histocompatibility Complex (MHC) combinations as tumor-specific peptides or proteins on the inside of cells. In a twenty-third embodiment, the T cell antigen coupler (TAC), a chimeric receptor that co-opts the endogenous TCR, will also promote a more efficient anti-tumor response and reduced toxicity when compared with HERV-K CAR-T.

In a twenty-fourth embodiment, the invention provides cancer cells overexpressing HERV-K. These cancer cells can be particularly good targets and good models for TCR, vaccine, peptide, shRNA, and other HERV-K directed drug therapies.

In a twenty-fifth embodiment, the invention provides a platform that enables functional matching TCR sequences acquired from a small number of homogenous HERV-K specific T cell (K-T cell) populations derived from a single clonally expanded K-T cell.

In a twenty-sixth embodiment, the invention provides HERV-K specific T cells (K-T cells) derived from tumor-infiltrating lymphocytes or peripheral blood mononuclear cells exhibiting secretion of IFNγ by ELISPOT.

In a twenty-seventh embodiment, the invention provides K-T TIL cells with cytotoxicity toward their autologous mammosphere cells.

In a twenty-eighth embodiment, the invention provides that forced overexpression of HERV-K with agents that induce expression of HERV-K by innate immune response (such as Poly I:C treatment) or LTR hypomethylation (such as by 5-Aza) provokes cancer cells to increase production of a target that would make them more susceptible to targeted therapy to include targeted immunotherapy.

In a twenty-ninth embodiment, the invention provides dendritic cells (DCs) transfected with HERV-K surface (SU) envelope (Env) protein. These cells have a much greater number of IFNγ spots than DCs transfected with a control GST protein, indicating a much greater immune response. When the peripheral blood mononuclear cells or TILs were also treated with anti-PD-L1, anti-CTLA-4, anti-LAG-3, and anti-TIM-3 antibodies the immune response was even stronger, especially using both anti-LAG-3 and anti-TIM-3 antibodies in comparison to both anti-PD-1 and anti-CTLA-4 antibodies. Thus, LAG-3 and TIM-3 exhaustion in the HERV K-T cells can be countered by anti-LAG3 and anti-TIM-3 therapy.

In one aspect, these findings support the concept that HERV-K triggers an immune response that can be complemented by immune checkpoint protein therapy. Therefore HERV-K effectively convert breast cancer from cold into hot tumors if it combines with the correct checkpoint blockade therapy partners.

In a thirtieth embodiment, the invention provides significantly increased percentages of CD8 T cells infiltrating tumors of mice inoculated with 4T1-pLVXKenv (4T1_K) cells and immunized with either HERV-K full-length surface protein (KSU) or full-length TM protein (KTM), in comparison to mice inoculated with cells transduced with vector only (4T1_C) and immunized with KSU or KTM. GST protein was a control antigen. Significantly decreased Treg cell percentages can be detected in tumors from mice inoculated with 4T1_K than with 4T1_C cells and immunized with KSU, a change not observed for KTM immunosuppressive protein immunization.

In a thirty-first embodiment, the invention provides increased macrophage, neutrophil, NK, NKT cells, and myeloid-derived suppressor cells (MDSC) in mice immunized with KSU than with KTM or GST, after challenge with tumor cells expressing HERV-K. These findings indicate that CD8 T cells play a role in killing tumor cells expressing HERV-K in vaccinated mice.

In a thirty-second embodiment, the invention provides reduced weight of pLVXKenv relative to pLVX tumors in mice immunized with KSU (50% reduced weight), showing the protective effect of KSU vaccination. This protective effect disappears in mice immunized with the TM (1.65-fold increased tumor weight). Thus, the immunosuppressive domain (ISD) of TM can prevent an immune response to the vaccine.

In a thirty-third embodiment, the invention provides that BALB/c female mice (6 weeks old) inoculated subcutaneously with 4T1_K (1×10⁵ cells) on day 0. Mice are treated with HERV-K surface protein (HERV-K SU) (100 μg), or with Amph-CpG (1.2 nmol) or CpG (1.2 nmol) on day 6, day 13 and day 19 after tumor inoculation (N=6/group). The tumor size can be monitored longitudinally throughout the study.

In a thirty-fourth embodiment, the invention provides three peptides bound consistently to HERV-K mAbs from several lots. These peptides are translated into HERV-K-specific vaccines, starting with peptide #135, which binds to all the anti-HERV-K mAbs.

In a thirty-fifth embodiment, the invention provides that transduction of breast cancer cells with the inventive shRNAenv inhibitor of HERV-K env mRNA showed synergy with standard of care therapy effects on cell proliferation and progression. Thus, the sensitivity of breast cancer cells toward anticancer agents can be greatly increased by a factor of at least 5 after KD of HERV-K.

In a thirty-sixth embodiment, the invention provides significantly reduced migration and invasion in MCF-7, HS578T cells, or MDA-MB-231 cells after treatment with paclitaxel or SRI-28731 (0.1 μM), or after KD of HERV-K.

In a thirty-seventh embodiment, the invention provides S phase arrest in MCF-7 and Hs578T breast cancer cell lines transduced with shRNAenv compared with control cells. G2 arrest occurs in the Hs578T cells treated with paclitaxel or SRI-28731, especially in the shRNAenv cells.

In a thirty-eighth embodiment, the invention provides a phosphoprotein array analysis of MCF-7 cells transduced with shRNAenv or with shRNAc and treated with SRI-28731 revealed STAT3 Y705, STAT3 S727, Hck, RSK1/2/3, AMPKα2 as the five major upregulated proteins, and ERK1/2, p38×, JNK1/2/3, c-Jun, and Lck as the five major downregulated proteins after HERV-K KD cells were treated with SRI-28731.

In another aspect, these phosphoprotein array data support the concept that HERV-K expression in cancer activates two important signaling pathways: MAPK/Ras and P13K/AKT.

In a thirty-ninth embodiment, the invention provides a HERV-K KD by shRNA. Visualization and Integrated Discovery (DAVID) pathway analysis revealed the most differentially expressed classes to be proteoglycans in cancer (proteoglycans were recently shown to be critical for HERV-K entry into cells), p53 signaling pathway and others. These data show that HERV-K expression is strikingly and closely associated with proteoglycans in cancer. HERV-K KD in cancer cells can have a very strong effect on expression of proteoglycans in these cells.

In a fortieth embodiment, the invention provides enhanced expression of HERV-K was detected in three paclitaxel-resistant breast cancer cell lines.

In a forty-first embodiment, the invention provides significantly increased serum levels of the reactive oxygen species hydrogen peroxide (H₂O₂) and malondialdehyde (MDA) but decreased serum levels of catalase (CAT) were observed in patients with breast cancer, especially in paclitaxel-resistant breast cancer patients.

In a forty-second embodiment, the invention provides that reactive oxygen species induces HERV-K expression, cancer cell proliferation, and cancer cell migration.

In a forty-third embodiment, the invention provides elevated expression of HERV-K mRNA in three breast cancer cell lines treated with H₂O₂ to achieve intracellular levels of reactive oxygen species that are positively associated with HERV-K expression.

In a forty-fourth embodiment, the invention provides that reactive oxygen species and chemotherapeutic agents regulate the expression of HERV-K, HIF-1α, P-RSK, P-ERK, and Ras.

In a forty-fifth embodiment, the invention provides cells treated with graded concentrations of H₂O₂ ranging from 1-50 μM showed enhanced expression of HERV-K, Ras, p-ERK, and HIF-1α proteins at H₂O₂ concentrations of 5 μM and 10 μM in the three breast cancer cell lines.

In a forty-sixth embodiment, the invention provides that reactive oxygen species increases biomarkers of EMT via induction of HERV-K expression.

In a forty-seventh embodiment, the invention provides HERV-K, p-MEK, and p-ERK, as well as expression favoring EMT markers such as E-cadherin and N-cadherin, vimentin and Slug in breast cancer cells treated with H₂O₂.

In a forty-eighth embodiment, the invention provides HERV-K as an upstream modulator of the Ras/ERK signaling pathway. HERV-K expression is stimulated by physiological levels of reactive oxygen species. Thus, reactive oxygen species (ROS) (5 μM to 10 μM) upregulates the expression of HERV-K, and HERV-K in turn induces EMT. In another aspect, these data indicate that reactive oxygen species (ROS) or HERV-K inhibitors or both can blockade the EMT that initiates invasion and metastasis of cancer cells.

In a forty-ninth embodiment, the invention provides PBMCs in vitro stimulated (IVS) with their autologous dendritic cells pulsed with KSU protein (K-T cells). The inventors observed an enhanced percentage of target cell lysis using CD8⁺ K-T effector cells, when compared with CD8⁺ T cells. The inventors also observed a decrease in lysis of target cells with HERV-K shRNAenv KD. Significantly increased killing of the PDX mammosphere cells by K-T cells was demonstrated, compared with T cell killing. A greater release of IFNγ cytokine and granzyme B was detected with increased concentrations of KSU used to pulse IVS cells.

In a fiftieth embodiment, the invention provides that administration of T cells pulsed with dendritic cells loaded with HERV-K (K-T cells) led to reduced tumor weights and decreased expression of cell signaling pathway intermediates that are integral to the formation and growth of cancer.

In a fifty-first embodiment, the invention provides the evaluation of expression of HERV-K in tumors and other organs by IHC or by FACS using anti-HERV-K 6H5 mAb. Significantly reduced expression of HERV-K Env protein was demonstrated in tumor or lung tissues of mice treated with K-T cells.

In a fifty-second embodiment, the invention provides reduced expression of MDM2 or CDK5 and increased expression of P53 correlates with decreased expression of HERV-K in mice treated with K-T cells compared with other cell therapies. Decreased expression of HERV-K Env protein, MDM2, p-ERK and Ras is further demonstrated by immunoblot in tumor tissues of mice treated with K-T cells.

In a fifty-third embodiment, the invention provides reduced weight of pLVXKₑₙᵥ relative to pLVX tumors in mice immunized with KSU (50% reduced weight), showing the protective effect of KSU vaccination. This protective effect disappears in mice immunized with the TM (1.65-fold increased tumor weight). Thus, the immunosuppressive domain (ISD) of TM can prevent an immune response to the vaccine.

In a fifty-fourth embodiment, the invention provides three Ras genes in humans, which are key molecular regulators controlling cell proliferation, transformation, differentiation, and survival. The HERV-K activates Ras genes using a mechanism not involving mutational activation of Ras.

In another aspect, the significance of these data is that HIF-1α, a key transcription factor activated by reactive oxygen species, and whose expression increases in breast cancer and indicates poor patient prognosis, is upregulated in tandem with HERV-K and Ras signaling pathway intermediates in several breast cancer cell lines. These data show that blocking HIF-1α expression via HERV-K KD can be an avenue for cancer therapy.

In a fifty-fifth embodiment, the invention provides combined cancer therapies that include but are not limited to combinations of (a) HERV-K humanized therapeutic antibodies or HERV-K fully human antibodies (1.5 mg/kg), (b) K-CAR, (c) K-BITE, (d) HERV-K shRNAs, locked nucleic acid-based antisense oligonucleotides, or CRISPR/Cas9 genome editing technology to knock down HERV-K gene expression, or (e) preventative or therapeutic HERV-K vaccines, including full-length and truncated HERV-K Env proteins and HERV-K Env peptides. Effective combined cancer therapies include full-length and truncated HERV-K Env proteins and HERV-K Env peptides, combined with factors including but not limited to (a) anti-ICP antibody, (b) cancer chemotherapy, (c) 5-zacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion, (f) induction of S or G2 phase cell cycle arrest, (g) inhibitors of PI3K/AKT/mTOR or MAPK/ERK signaling pathways, or (f) signal transduction to HIF1a.

### BRIEF DESCRIPTION OF THE DRAWINGS

FIG. 1. illustrates the baseline immune status in relation to HERV-K status in breast cancer patients: combined HERV-K and immune checkpoint assays. Expression of soluble immune checkpoint proteins was determined by Luminex assay in breast cancer patients including DCIS and aggressive breast cancer vs. normal donors. A striking finding was a significantly enhanced expression of six circulating ICPs in the plasma of breast cancer patients. See FIG. 1A. A further finding was a marked drop in immune checkpoint protein levels in patients at six months (FIG. 1B; Timepoint 2) or eighteenth months (data not shown) post-surgery vs. pre-surgery (Timepoint 1). Importantly, a positive association between soluble ICP molecule levels and HERV-K antibody titers induced by HERV-K expression in the tumor was observed (FIG. 1(C)), suggesting that HERV-K antibody titers would influence ICP levels in breast cancer. The expression of HERV-K can thus control immune responses of breast cancer patients.

FIG. 2 is a bar graph showing ELISpot and flow analysis of patient PBMCs after pulsing. ELISpot plates were coated with IFN-γ capture antibody one day before the experiment and blocked with medium containing 10% FBS for 30 min. PBMCs from patients #407, 441, 436 and 460 and both PBMCs and TILs from patients 443 and 438 were pulsed for one week with dendritic cells loaded with KSU and GST. 5×10⁴ cells T cells and protein pulsed dendritic cells (30:1) were seeded in each well of a 96-well plate with each treatment done in triplicate. KSU and GST were delivered into dendritic cells using the Bioporter protocol. After twenty hours, cells were exposed to a detection antibody for two hours, followed by addition of streptavidin-HRP for one hour and color development with TMB. Flow cytometry was used to test for expression of the following T cell and exhaustion markers: CD3, CD4, CD8, PD-1, CTLA-4, LAG-3, and TIM-3. The breast cancer types were: 407: IMC, IDC, DCIS; 441: IDC; 436: DCIS; 460: IDC; 443: IDC; 438: IDC.

FIG. 3. FIG. 3A is a bar graphs showing percentages of CD33, CD3, and CD19⁺ cells quantified in huCD45⁺ cells obtained at four weeks post-inoculation of TNBC PDX cells, and in the MDA-MB-231 HTM model at seven weeks post-inoculation, with co-implantation of CD34⁺ hematopoietic stem cells. FIG. 3C is an immunofluorescence staining used to detect the expression of HERV-K using anti-HERV-K mAb 6H5 in an MDA-MB-231 tumor obtained from an HTM. F-actin was used as the control (two left panels). huCD3⁺ cells were also detected in tumor tissues (two right panels). Anti-HERV-K antibody titers were detected by ELISA in HTM models inoculated with MDA-MB-231 (HTM1) or MDA-MB-468 (HTM2) and with HM1 and HM2 immunized with HERV-K SU Env protein using anti-human IgG mAb.

FIG. 4. ELISPOT analysis of IFNγ secretion by T cells from patients 390 (FIG. 4A) and 351 (FIG. 4B). Increased IFNγ secretion by DCs pulsed with HERV-K SU protein or GST (control protein) with or without anti-PD-L1, CTLA-4, LAG-3, TIM-3, or combined LAG-3⁺ TIM-3 mAbs. FIG. 4C. Classes of T cells were determined by flow cytometry. LAG-3⁺, PD-1⁺, and TIM-3⁺ percentages were significantly increased in CD8⁺ T when compared to CD4⁺ T cells of K-specific T cells treated with anti-LAG-3 antibody. D) The TIM-3⁺ CD8⁺ fraction showed a more significant decrease than the TIM-3⁺ CD4⁺ fraction of K-specific T cells treated with anti-LAG-3 plus anti-TIM-3 mAbs.

FIG. 5. Significantly increased tumor CD8 T cell infiltration was demonstrated in mice inoculated with 4T1_K and immunized with KTM or KSU than in mice inoculated with 4T1_C cells. FIG. 5A. However, significantly decreased Treg cells were detected in tumors from mice inoculated with 4T1_K than with 4T1_C cells, and immunized with KSU, a change not observed for KTM protein immunization. GST protein was used as control. FIG. 5C. Multiple cytokine arrays showed increased IFNγ and TNF-α in spleens from mice inoculated with 4T1_K or 4T1_C cells and immunized with KSU than when immunized with KTM protein. FIG. 5D. Innate immune responses were determined. Increased macrophage, neutrophil, NK, NKT, or MDSC cells were demonstrated in mice inoculated with 4T1_K than with 4T1_C cells and immunized with KSU but not with KTM or GST protein. FIG. 5E. Increased tumor weights were observed in mice immunized with KSU, KTM, or GST protein, then challenged with CT26_K cells, and treated with anti-CD8 antibody. FIG. 5F.

FIG. 6. C57BL/6 mice were immunized with CDN (15 μg) plus 25 μg/mouse of GST, KSU-GST, or TM-GST fusion proteins on day 1, day 14, and day 28, and inoculated with 3×10⁵ B16F10 pLVX-Kenv or B16F10 pLVX cells following the last tumor antigen immunization. FIG. 6A. Tumor weight was compared among groups. FIG. 6B. ELISA was used to determine anti-HERV-K SU antibody titers among groups. The inventors determined the status of CD3⁺ CD4⁺FoxP3 FIG. 6C, CD3⁺ CD8 (FIG. 6D), and NK (FIG. 6E) of tumors of mice immunized with GST, KSU or TM, and challenged with B16F10 cells transduced with HERV-K TM (Kenv) or a control plasmid with no insert (pLVX).

FIG. 7. Peptide mapping of the full-length HERV-K env gene sequence (144 peptides). Antibodies from several lots bound to the 15-mer peptides #58, #88, and #135 (red arrows).

FIG. 8. Effect of HERV-K knockdown on growth of breast cancer cells treated with paclitaxel or SRI-28731. Significantly reduced cell proliferation was observed for MDAMB-231, Hs578T, and MCF-7 cells, after HERV-K KD by shRNAenv and treatment with paclitaxel (FIG. 8A) or SRI-28731 (FIG. 8B) compared with their parent cells or shRNAc-transduced cells. Enhanced sensitivity of cell lines toward drugs was demonstrated in Hs578T and MCF-7 breast cancer cell lines transduced with shRNAenv compared with cells transduced with shRNAc or parent cells. See FIG. 8C.

FIG. 9. HERV-K regulates drug sensitivity, migration, and invasion of breast cancer cell lines. FIG. 9A. Enhanced sensitivity of cell lines toward SRI-28731 was demonstrated in MCF-7 breast cancer cell lines transfected with shRNAenv compared with cells transfected with shRNAc or parent cells. Significantly enhanced sensitivities were demonstrated toward levels ranging from 10 μM to 0.0032 μM of SRI-28731 in MCF-7 cells treated with shRNAenv. FIG. 9B. Enhanced sensitivity of cell line toward doxorubicin (DOX) was demonstrated in MDA-MB-231 breast cancer cells with KD of the HERV-K env gene.

FIG. 10. Significantly reduced migration and invasion of MCF-7 (FIG. 10A), MDA-MB-231 (FIG. 10B), or Hs578T (FIG. 10C) cells after treatment with paclitaxel or SRI-28731 (0.1 μM), or after KD of HERV-K (FIG. 10D). Images of cell invasion are shown. Synergy between HERV-K KD and drug treatment was observed in the three cell lines. * P>0.01; ** P>0.001; *** P>0.0001; and **** P<0.0001.

FIG. 11. Phosphoprotein array analysis of MCF-7 breast cancer cells treated with SRI-28731 after shRNA knockdown of HERV-K.

FIG. 12. FIG. 12A shows a Venn diagram of significant differentially expressed genes (FDR<0.05) due to HERV-K KD in MCF-7 and MDA-MB-231 cell lines. FIG. 12B. Heatmap depicting the unsupervised hierarchical clustering of the top 20 (FDR<0.05) differentially expressed genes in the MDA-MB-231 cell line. These genes are a subset of 733 common genes between the 2 cell lines, as seen in the Venn diagram. RNA expression is Log 2 fold change over control; red to green color gradation is based on the ranking of each condition from minimum to maximum. FIG. 12C. KEGG pathways analysis of 733 common genes by DAVID. Numbers beside the bars represent gene members of the respective KEGG pathway present in 733 common genes.

FIG. 13. Enhanced expression of HERV-K in paclitaxel-resistant breast cancer cells. FIG. 13B. Overexpression of HERV KSU RNA was observed in two TNBC cell lines (MDA-MB-231 and Hs578T) with paclitaxel exposure. P: parent cells, Tax: paclitaxel-resistant breast cancer cells. β-actin was used as control. Middle panel: Overexpression of HERV-K Env protein was detected in paclitaxel-resistant breast cancer cells (red scan) relative to their parent cells (green scan). The isotype control is colored gray. FIG. 13C. Significantly increased proliferation was demonstrated in paclitaxel-resistant MDA-MB-231, Hs578T, and MCF-7 cells compared with parent cells. Data are presented as mean±SD.

FIG. 14. Evaluation of serum levels of hydrogen peroxide, MDA, and catalase activity in BC patients with drug-resistance. Significantly increased serum levels of H₂O₂ and MDA, and decreased serum levels of catalase were observed in BC patients and in paclitaxel-resistant patients compared with normal female donors (ND).

FIG. 15. FIG. 15A shows that significantly increased intracellular levels of H₂O₂ were demonstrated in drug-resistant cell lines. FIG. 15B shows that significant positive association of HERV-K expression with intracellular levels of reactive oxygen species, as assessed using Pearson's correlation. Significantly increased proliferation (FIG. 31C) and migration (FIG. 15D) was observed in BC cells treated with H₂O₂ (5 μM).

FIG. 16 FIG. 16A shows the expression of HERV-K and activities of HIF-1a and Ras/ERK pathways in drug-resistant or H₂O₂ treated BC cell lines. FIG. 16B. However, elevated concentrations of H₂O₂ (25 μM and 50 μM) resulted in decreased expression of HERV-K and corresponding decreased HIF-1a and Ras/ERK signaling.

FIG. 17. H₂O₂ alters expression of HERV-K and cancer signaling pathways in a time-dependent manner. FIG. 17(A). Higher expression of HERV-K was detected in BC cells treated with H₂O₂ for approximately 18 hours. FIG. 17(B) Treatment of HERV-K KD BC cell lines with H₂O₂ (5 μM) longer than 24 hours reversed the shRNAenv-induced block in expression of HERV-K, HIF-1α, P-RSK, P-ERK, and Ras.

FIG. 18. CTL assays were used to determine the cytotoxicity of K-T cells toward BC cells at various ratios of effector to target. FIG. 18A. Significantly greater lysis was demonstrated in MDA-MB-231cell line using K-T cells from patients 277, 278, and 243 (top panel) and three normal donors (bottom panel) at an effector/target ratio of 20:1. Enhanced specific lysis was observed in MDA-MB-231 cells using CD8⁺ K-T cells from patient 243 with CD4⁺ T depletion, compared to no depletion of CD4⁺ T cells (top-right panel). Decreased specific lysis was observed in MDA-MB-231 cells with KD of HERV-K by shRNAenv (bottom-right). FIG. 18B. CTL assays were used to determine the cytotoxicity of K-T cells toward mammosphere cells at various ratios of effector to target. Significantly greater lysis of mammospheres was observed by K-T cells than by T cells generated from a normal donor at an effector/target ratio of 1:1, 5:1, and 25:1.

FIG. 19. An ELISA assay was used to detect cytokine release from breast cancer cells treated with K-T or control T cells. Significantly enhanced Granzyme B (FIG. 19A) and IFN-γ (FIG. 19B) release into the culture media obtained from a breast cancer patient (243) or a normal donor (ND427478) was demonstrated in target cells treated with K-T generated by pulsing with 10, 20, or 40 μg/ml of KSU protein. A greater release of cytokine was detected with increased concentration of KSU for both normal donors and breast cancer patients. PMA/IONO was used as a positive control to stimulate maximal cytokine release.

FIG. 20. Significantly reduced tumor weight (FIG. 20A) and growth (FIG. 20B) was observed in mice treated with K-T cells compared to treatment with other controls including T cells or PBS.

FIG. 21A. Greater numbers of metastatic MDA-MB-231 cells (green color) were observed in brain, lung, liver, kidney, spleen, and bone biopsies of mice treated with PBS than with T cells (left panel). FIG. 21B. Significantly reduced numbers of metastatic foci were observed in mice treated with K-T cells (right panel).

FIG. 22. Significantly reduced expression of HERV-K was observed in tumor or lung tissues obtained from mice treated with K-T cells compared to other treatments, as detected by flow cytometry.

FIG. 23. RT-PCR was used to determine the expression of HERV-K env gene SU and TM in 4T1 or B16F10 cells stably transfected with pLVXKenv (full-length HERV-K SU+TM) or pLVX and analyzed for expression using HERV-K type 1 SU or TM primers. FIG. 23A. BALB/c mice were inoculated with 3×10⁵ 4T1pLVXKenv or 4T1pLVX cells on day 0. These mice were then treated: FIG. 23B with CpG (1.24 nmol), HERV-K SU protein (10 μg, 20 μg, and 20 μg), or CpG+ HERV-K SU protein on days 6, 13, and 19. Tumor weights were compared at week 4 post tumor cell inoculation; (FIG. 23C) with Aza (0.5 mg/kg) daily for five days in two weekly cycles, anti-PD-1 (200 μg per mouse every four days; four treatments), or Aza+anti-PD-1. Mice treated with PBS were used as controls, and tumor weights were compared among groups; and (FIG. 23D) with anti-HERV-K antibody (6H5; once) and with IL2 (for five days). Significantly decreased T regulatory (Treg) cells were detected in tumor cells stably transfected with pLVXKenv than with pLVX controls in mice treated with IL-2, 6H₅, and IL-2 plus 6H5. (*=P<0.05, **=P<0.01, and ***=P<0.001).

FIG. 24. Knockdown (KD) of HERV-K env gene in MCF-7 BC cells with an shRNA targeting gene (shRNAenv), or MCF-7 cells treated with a control shRNA (shRNAc). MCF-7 cells were also transduced with a vector that overexpresses HERV-K (pLVXKenv) and a pLVXc control vector. Expression of Ras was determined by RT-PCR in MCF-7 parent cells. Downregulation of K-Ras N-Ras and H-Ras was observed in HERV-K KD cells, and upregulated expression of Ras was observed in the pLVXKenv HERV-K overexpressing cells. Immunoblot assays showed increased levels of HIF-1alpha, p-RSK, HERV-K, p-ERK 1 or 2, and Ras protein in MCF-7 shRNAc and MCF-7 shRNAₑₙᵥ cells transduced with Kenv or Kmut (a mutation of HERV-K env not recognized by shRNA). Increased expression of KRas was also detected by RT-PCR in immortalized, nontumorigenic MCF-10AT breast cells stably transfected with pLVXKenv, compared to cells transfected with pLVX (vector only). Enhanced Ras activity was further detected in MCF-10AT+pLVXKenv cells. Negative and Positive are controls included in the assay kit.

FIG. 25A. Increased expression of KRas was also detected by RT-PCR in immortalized, nontumorigenic MCF-10A breast cells stably transfected with pLVXKenv, compared to cells transfected with pLVX (vector only). Enhanced (FIG. 25B) cell proliferation and Ras activity (FIG. 25C) was further detected in MCF-10A+pLVXKenv cells. Negative and Positive are controls included in the assay kit. Enhanced transformation was observed in MCF-10A+pLVXKenv cells.

### DETAILED DESCRIPTION OF THE INVENTION

### Utility of the Invention

This specification provides methods for generated a humanized anti-HERV-K antibody. Anti-tumor effects of hu6H5 were demonstrated in vitro and in vivo.

This invention provides methods for treating patients suffering from cancer. In a fifty-sixth embodiment, the invention provides to a method of treating cancer comprising administering a therapeutic humanized anti-HERV-K antibody or a fusion thereof consisting of a CAR, a BiTE or an ADC, a cancer vaccine, and optionally combine with one or more immune checkpoint blockers. Each of these therapeutics individually target the immune system. In a fifty-seventh embodiment, the methods of the invention inhibit metastases. In a fifty-eighth embodiment, the methods of the invention reduce tumor size. In a fifty-ninth embodiment, the methods of the invention inhibit the growth of tumor cells. In a sixtieth embodiment, the methods of the invention detect cancer and cancer metastasis.

This specification provides methods for isolated HERV-K viral particle from cancer patient blood and tumor tissues. The HERV-K viral particles were presented in patients with IDC and adenocarcinoma. Full lengths of gag, pol, and env with higher RT activities were demonstrated in these samples.

RT-PCR was used to determine if viral particles express full length gag, pol and env genes. Full length gag, pol, and env PCR products were further sequenced and the sequencing results were blasted on https://blast.ncbi.nlm.nih.gov/Blast.cgi. Putative conserved domains including gag, pol, and env were detected.

An env gene obtained from the viral particles can function in tumor development, especially tumor metastasis due to upregulated multiple oncogenes and downregulated multiple tumor suppressed genes.

This specification provides that HERV-K env gene is an oncogene not only enhanced tumor viability, but also causes metastasis to other organs in vivo.

This specification provides methods for treating patients suffering from cancer. In a sixty-first embodiment, the invention provides to a method of treating cancer comprising administering a therapeutic humanized anti-HERV-K antibody or a fusion thereof, a cancer vaccine, and optionally combine with one or more immune checkpoint blockers. Each of these therapeutics individually target the immune system. In a sixty-second embodiment, the invention provides a method of treating cancer comprising administering a TCR construct or a TAC construct recognizing tumor-specific HERV-K protein fragment/Major Histocompatibility Complex (MHC) combinations presented on the surface of the tumor cell. In a sixty-third embodiment, the methods of the invention prolong survival of subjects with cancer. In a sixty-fourth embodiment, the methods of the invention inhibit metastases. In a sixty-fifth embodiment, the methods of the invention reduce tumor size. In a sixty-sixth embodiment, the methods of the invention inhibit the growth of tumor cells. In a sixty-seventh embodiment, the methods of the invention detect cancer and cancer metastasis.

### Definitions

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skill in the molecular biology art. For any apparent discrepancy between the meaning of a term in the art and a definition provided in this specification, the meaning provided in this specification shall prevail.

5-Aza has the biomedical art-recognized meaning of 5-azacytidine.

6H5 in this specification means a particular anti-HERV-K monoclonal antibody described herein.

About has the plain meaning, which varies depending on the context in which the term is used. If there are uses of which are not clear to persons of ordinary skill in the biomedical art given the context in which it is used, about will mean up to plus or minus 10% of the value.

Antibody-drug conjugate (ADC) has the biomedical art-recognized meaning of highly potent biological drugs built by attaching a small molecule anticancer drug or another therapeutic agent to an antibody, with either a permanent or a labile linker. The antibody targets a specific antigen only found on target cells.

Antigen Presenting Cells (APC) has the biomedical art-recognized meaning of cells that can process a protein antigen, break it into peptides, and present it in conjunction with class II Major Histocompatibility Complex molecules on the cell surface where it may interact with appropriate T cell receptors.

Artificial Antigen Presenting Cells (aAPC) has the biomedical art-recognized meaning of platforms of antigen presenting cells that are engineered for T-cell activation.

B7 family has the biomedical art-recognized meaning of inhibitory ligands with undefined receptors. The B7 family encompasses B7-H3 and B7-H4, both upregulated on tumor cells and tumor infiltrating cells. The complete hB7-H3 and hB7-H4 sequence can be found under GenBank Accession Nos. Q5ZPR3 and AAZ17406, respectively.

BiTE has the biomedical art-recognized meaning of a bispecific T cell engager. A BiTE means a recombinant bispecific protein that has two linked scFvs from two different antibodies, one targeting a cell-surface molecule on T cells (for example, CD38) and the other targeting antigens on the surface of malignant cells. The two scFvs are linked together by a short flexible linker. The term DNA-encoded BiTE (DbiTE) includes any BiTE-encoding DNA plasmid that can be expressed in vivo.

Cancer antigen or tumor antigen has the biomedical art-recognized meaning of (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

Cancer Vaccine has the biomedical art-recognized meaning of a treatment that induces the immune system to attack cells with one or more tumor associated antigens. The vaccine can treat existing cancer (e.g., therapeutic cancer vaccine) or prevent the development of cancer in some individuals (e.g., prophylactic cancer vaccine). The vaccine creates memory cells that will recognize tumor cells with the antigen and therefore prevent tumor growth. In some embodiments, the cancer vaccine comprises an immunostimulatory oligonucleotide.

CG Oligodeoxynucleotides (CG ODNs), also referred to as CpG ODNs, have the biomedical art-recognized meaning of short single-stranded synthetic DNA molecules that contain a cytosine nucleotide (C) followed by a guanine nucleotide (G). In some embodiments, the immunostimulatory oligonucleotide is a CG ODN.

Combination Therapy has the biomedical art-recognized meaning and embraces administration of each agent or therapy in a sequential manner in a regimen that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements can be administered at separate times and/or by different routes, but which act in combination to provide a beneficial effect by co-action or pharmacokinetic and pharmacodynamics effect of each agent or tumor treatment approaches of the combination therapy.

Circulating Tumor Cells (CTCs) has the biomedical art-recognized meaning of cancer cells that split away from the primary tumor and appear in the circulatory system as singular units or clusters.

Cytolytic T Cell or Cytotoxic T Cell (CTL) has the biomedical art-recognized meaning of a type of immune cell that can kill certain cells, including foreign cells, cancer cells, and cells infected with a virus. CTLs can be separated from other blood cells, grown in the laboratory, and then given to a patient to kill cancer cells. A CTL is a type of white blood cell and a type of lymphocyte.

Cytotoxic T Lymphocyte Associated Antigen-4 (CTLA-4) has the biomedical art-recognized meaning of a T cell surface molecule and is a member of the immunoglobulin superfamily. This protein downregulates the immune system by binding to CD80 and CD86. The term CTLA-4 as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. P16410.

Dendritic Cell (DC) has the biomedical art-recognized meaning of a special type of immune cell that boosts immune responses by showing antigens on its surface to other cells of the immune system.

Ductal Carcinoma In Situ (DCIS) has the biomedical art-recognized meaning of the presence of abnormal cells inside a milk duct in the breast.

Derived From a Designated Polypeptide or Protein has the biomedical art-recognized meaning of the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the in the molecular biological art as having its origin in the sequence. Polypeptides derived from another peptide can have one or more mutations relative to the starting polypeptide, e.g., one or more amino acid residues which were substituted with another amino acid residue, or which has one or more amino acid residue insertions or deletions. A polypeptide can comprise an amino acid sequence which is not naturally occurring. Such variants necessarily have less than 100% sequence identity or similarity with the starting molecule. In some embodiments, the peptides are encoded by a nucleotide sequence. Nucleotide sequences of the invention can be useful for several applications, including cloning, gene therapy, protein expression and purification, mutation introduction, DNA vaccination of a host in need thereof, antibody generation for, e.g., passive immunization, PCR, primer and probe generation, and the like.

Effector Cell has the biomedical art-recognized meaning of an immune cell which is involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express specific Fc receptors (FcRs) and carry out specific immune functions.

Epithelial-Mesenchymal Transition (EMT) has the biomedical art-recognized meaning of a biological process in which epithelial cells lose cell-cell junctions, apical-basal polarity, epithelial markers, and acquire cell motility, a spindle-cell shape, and mesenchymal markers.

Env has the biomedical art-recognized meaning of the viral envelope protein. Likewise, env has the biomedical art-recognized meaning of the corresponding viral envelope RNA.

Epitope has the biomedical art-recognized meaning of determinant capable of specific binding to an antibody. Epitopes usually consist of surface groupings of molecules such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents. The epitope can comprise amino acid residues directly involved in the binding (also called immunodominant component of the epitope) and other amino acid residues, which are not directly involved in the binding, such as amino acid residues which are effectively blocked by the specifically antigen binding peptide (in other words, the amino acid residue is within the footprint of the specifically antigen binding peptide).

Fluorescence Activated Cell Sorting (FACS) the biomedical art-recognized meaning.

Gold Nanoparticles (GNP) has the biomedical art-recognized meaning of small gold particles with a diameter of 1 to 100 nm which, once dispersed in water, are also known as colloidal gold.

Hydrogen Peroxide (H₂O₂) has the biomedical art-recognized meaning of a peroxide and oxidizing agent with disinfectant, antiviral and anti-bacterial activities. H₂O₂ is an endogenous reactive oxygen species.

Human Endogenous Retrovirus (HERV) has the biomedical art-recognized meaning and includes any variants, isoforms and species homologs of endogenous retroviruses which are naturally expressed by cells or are expressed on cells transfected with endogenous retroviral genes. HERV is a retrovirus that is present in the form of proviral DNA integrated into the genome of all normal cells and is transmitted by Mendelian inheritance patterns. HERV-X, where X is an English letter, has the biomedical art-recognized meaning of other families of HERVs, which have been further classified on the basis of the tRNA that binds to the viral primer binding site (PBS) to prime reverse transcription. HERV-K thus implies a provirus or ERV lineage that uses a lysine tRNA, no matter their relationship to one another. Common HERV families include HERV-T (a representative small to medium-sized HERV family), HERV-L (the oldest family that infected a common ancestor of mammals), HERV-H (the most abundant family in humans), HERV-W (which has been co-opted by host to function in placenta formation); and HERV-K (the only family for which a functional infectious virus has been reconstructed in vitro, and which is capable of producing retroviral particles).

Human Endogenous Retrovirus (HERV) has the biomedical art-recognized meaning and includes members of the HERV-K family of endogenous retroviruses. HERV-K is expressed on many tumor types, including, but not limited to, melanoma, breast cancer (Wang-Johanning et al., (2003)), ovarian cancer (Wang-Johanning et al., (2007)), lymphoma, and teratocarcinoma. Infected cells, including those infected by HIV, also express HERV-K. This provides an attractive opportunity that one CAR design targeting HERV-K may be used to treat a variety of cancers and infections.

Humanized Mice (HM) has the biomedical art-recognized meaning of a general term that refers to a mouse that has been engrafted with something from a human.

Human Tumor Mice (HTM) has the biomedical art-recognized meaning of human cancer cells or cell lines have been xenografted into immunodeficient mice to create human tumor/mouse chimeras.

hTAb has the biomedical art-recognized meaning of a fully human tumor antibody.

Immune Checkpoint (ICP) has the biomedical art-recognized meaning of immune checkpoint. ICP molecules act as gatekeepers of immune responses.

Invasive Ductal Carcinoma (IDC) has the biomedical art-recognized meaning of cancer that occurs when abnormal cells growing in the lining of the milk ducts change and invade breast tissue beyond the walls of the duct.

Immunohistochemistry (IHC) has the biomedical art-recognized meaning of a process that uses antibodies to detect the location of proteins and other antigens in tissue sections.

Invasive Lobular Carcinoma (ILC) has the biomedical art-recognized meaning of breast cancer that begins in one of the glands that make milk, called lobules, and spreads to other parts of the breast.

Immune Cell has the biomedical art-recognized meaning of a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

Immune Checkpoint Blocker has the biomedical art-recognized meaning of a molecule that totally or partially reduces, inhibits, interferes with, or modulates one or more checkpoint proteins. In some embodiments, the immune checkpoint blocker prevents inhibitory signals associated with the immune checkpoint. In some embodiments, the immune checkpoint blocker is an antibody, or fragment thereof that disrupts inhibitory signaling associated with the immune checkpoint. In some embodiments, the immune checkpoint blocker is a small molecule that disrupts inhibitory signaling. In some embodiments, the immune checkpoint blocker is an antibody, fragment thereof, or antibody mimic, that prevents the interaction between checkpoint blocker proteins, e.g., an antibody, or fragment thereof, that prevents the interaction between PD-1 and PD-L1. In some embodiments, the immune checkpoint blocker is an antibody, or fragment thereof, that prevents the interaction between CTLA-4 and CD80 or CD86. In some embodiments, the immune checkpoint blocker is an antibody, or fragment thereof, that prevents the interaction between LAG3 and its ligands, or TIM-3 and its ligands. The checkpoint blocker can also be in the form of the soluble form of the molecules themselves or variants thereof.

Immune Checkpoint has the biomedical art-recognized meaning of costimulatory and inhibitory signals that regulate the amplitude and quality of T cell receptor recognition of an antigen. In some embodiments, the inhibitory signal may be the interaction between PD-1 and PD-L1; the interaction between CTLA-4 and CD80 or CD86 to displace CD28 binding; the interaction between LAG3 and Major Histocompatibility Complex class II molecules; the interaction between TIM3 and galectin 9; or some other interaction known in the biomedical art.

Immunostimulatory Oligonucleotide has the biomedical art-recognized meaning of an oligonucleotide that can stimulate, induce or enhance an immune response.

In Vivo has the biomedical art-recognized meaning of processes that occur in a living organism. The term mammal or subject or patient as used herein includes both humans and non-humans and includes, but is not limited to, humans, non-human primates, canines, felines, rodents, bovines, equines, and pigs.

Inhibits Growth, when referring to cells, such as tumor cells, has the biomedical art-recognized meaning and includes any measurable decrease in the cell growth when contacted with HERV-K specific therapeutic agents as compared to the growth of the same cells not in contact with the HERV-K specific therapeutic agents, e.g., the inhibition of growth of a cell culture. Such a decrease in cell growth can occur by a variety of mechanisms exerted by the anti-HERV-K agents, either individually or in combination, e.g., apoptosis.

Immunosuppressive Domain (ISD) has the biomedical art-recognized meaning.

In Vitro Stimulation (IVS) and In Vitro Stimulated has the biomedical art-recognized meaning of stimulation of T cells of cancer patients with the autologous tumor cell line.

K-CAR or HERV-Kₑₙᵥ CAR has the biomedical art-recognized meaning of a HERV-K envelope gene (surface or transmembrane) chimeric antigen receptor (CAR) genetic construct. The term HERV-Kenv CAR-T cells or K-CAR-T cells has the biomedical art-recognized meaning of T cells that were transduced with a K-CAR or HERV-Kenv CAR lentiviral or Sleeping Beauty expression system.

K-T cell in this specification means HERV-K specific T cell produced by exposure to HERV-K pulsed human dendritic cells, which are potent antigen presenting cells.

KD in this specification means knockdown, usually by an shRNA.

KSU in this specification means HERV-K envelope surface fusion protein.

KTM in this specification means HERV-K Env transmembrane protein.

Linked, Fused, or Fusion, are used interchangeably in this specification to mean the joining together of two more elements or components or domains, by whatever means including chemical conjugation or recombinant means. Methods of chemical conjugation, e.g., using heterobifunctional crosslinking agents, are known in the biomedical art.

Linker or Linker Domain has the biomedical art-recognized meaning of a sequence which connects two or more domains in a linear sequence, e.g., a humanized antibody targeting HERV-K and an antibody targeting a T cell protein. The constructs suitable for use in the methods disclosed herein can use one or more linker domains, such as polypeptide linkers.

Lymphocyte Activation Gene-3 (LAG3) is an inhibitory receptor associated with inhibition of lymphocyte activity by binding to Major Histocompatibility Complex class II molecules. This receptor enhances the function of Treg cells and inhibits CD8⁺ effector T cell function. LAG3 as used herein includes human LAG3 (hLAG3), variants, isoforms, and species homologs of hLAG3, and analogs having at least one common epitope. The complete hLAG3 sequence can be found under GenBank Accession No. P18627.

Mammosphere has the biomedical art-recognized meaning of breast or mammary cells cultured under non-adherent non-differentiating conditions that form discrete clusters of cells.

MDA-MB-231 pLVXC or 231-C in this specification means MDA-MB-231 cells that were transduced with pLVXC.

MDA-MB-231 pLVXK or 231-K in this specification means MDA-MB-231 cells that were transduced with pLVXK.

Nucleic Acid has the biomedical art-recognized meaning of deoxyribonucleotides or ribonucleotides and polymers thereof in either single-stranded or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues. See Batzer et al., Nucleic Acid Res., 19, 5081 (1991); Ohtsuka et al., Biol. Chem., 260, 2605-2608 (1985); and Rossolini et al., Mol. Cell. Probes, 8, 91-98 (1994). For arginine and leucine, modifications at the second base can also be conservative. The term nucleic acid is used interchangeably with gene, cDNA, and mRNA encoded by a gene.

Peripheral Blood Mononuclear Cell (PBMC) has the biomedical art-recognized meaning of a variety of specialized immune cells that work together to protect the body from harmful pathogens. PBMCs act as a line of defense from infection and disease.

Patient-Derived Xenograft (PDX) has the biomedical art-recognized meaning. A PDX is typically produced by transplanting human tumor cells or tumor tissues into an immunodeficient murine model of human cancer.

Percent Identity, in the context of two or more nucleic acid or polypeptide sequences, has the biomedical art-recognized meaning that two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent identity can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequences relative to the reference sequence, based on the designated program parameters. Optimal alignment of sequences for comparison can be conducted, e.g., by the local homology algorithm of Smith & Waterman, Adv. Appl. Math., 2, 482 (1981), by the homology alignment algorithm of Needleman & Wunsch, J. Mol. Biol., 48, 443 (1970), by the search for similarity method of Pearson & Lipman, Proc. Natl. Acad. Sci., U.S.A., 85, 2444 (1988), by computerized implementations of these algorithms (GAP, BESHERV-KIT, FASTA, and HERV-KASTA in the Wisconsin Genetics Software Package, Genetics Computer Group, 575 Science Dr., Madison, WI, USA), or by visual inspection. One example of an algorithm that is suitable for determining percent sequence identity and sequence similarity is the BLAST algorithm, which is described in Altschul et al., J. Mol. Biol., 215, 403-410 (1990). Software for performing BLAST analyses is publicly available through the National Center for Biotechnology Information website.

Pharmaceutically Acceptable has the biomedical art-recognized meaning of those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

pLVXC in this specification means a control expression vector.

pLVXK in this specification means an HERV-K expression vector.

pLVXKₑₙᵥ in this specification means the pLVX vector that expresses the full length HERV-K envelope protein, both extracellular surface (SU) and transmembralTM) domains.

Polypeptide Linker has the biomedical art-recognized meaning of a peptide or polypeptide sequence (e.g., a synthetic peptide or polypeptide sequence) which connects two or more domains in a linear amino acid sequence of a polypeptide chain. Such polypeptide linkers can provide flexibility to the polypeptide molecule. The polypeptide linker can be used to connect (e.g., genetically fuse) one or more Fc domains and/or a drug.

Programmed Death Ligand-1 (PD-L1) has the biomedical art-recognized meaning of one of two cell surface glycoprotein ligands for PD-1 (the other being PD-L2) that downregulates T cell activation and cytokine secretion upon binding to PD-1. PD-L1 as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

Programmed Death-1 (PD-1) receptor has the biomedical art-recognized meaning of an immuno-inhibitory receptor belonging to the CD28 family. PD-1 is expressed predominantly on previously activated T cells in vivo, and binds to two ligands, PD-L1 and PD-L2. PD-1 as used herein includes human PD-1 (hPD-1), variants, isoforms, and species homologs of hPD-1, and analogs having at least one common epitope with hPD-1. The complete hPD-1 sequence can be found under GenBank Accession No. AAC51773.

Recombinant Host Cell (or simply Host Cell) has the biomedical art-recognized meaning of a cell into which an expression vector was introduced. Such terms refer not only to the particular subject cell, but also to the progeny of such a cell. Because some modifications can occur in succeeding generations due to either mutation or environmental influences, such progeny might not, in fact, be identical to the parent cell, but are still included within the scope of host cell as used herein. Recombinant host cells include, for example, transfectomas, such as CHO cells, HEK293 cells, NS/0 cells, and lymphocytic cells.

Reactive Oxygen Species (ROS) has the biomedical art-recognized meaning of a type of unstable molecule that contains oxygen and that easily reacts with other molecules in a cell.

Reverse Transcriptase (RT) enzyme activity has the biomedical art-recognized meaning of an enzyme-mediated process that is responsible for the reverse transcription of retroviral single-stranded RNA into double-stranded DNA.

Single Chain Variable Fragment (scFv) has the biomedical art-recognized meaning of a particular kind of antibody fragment. A scFv is a fusion protein of the variable regions of the heavy (VH) and light chains (VL) of immunoglobulins, connected with a short linker peptide of ten to about 25 amino acids.

shRNA has the biomedical art-recognized meaning of short hairpin RNA or small hairpin RNA.

shRNAc has the biomedical art-recognized meaning of a scrambled shRNA control nucleotide sequence.

shRNAenv has the biomedical art-recognized meaning of an shRNA that targets the envelope gene of HERV-K.

SU in this specification means the HERV-K surface protein.

Sufficient amount or amount sufficient to means an amount sufficient to produce a desired effect, e.g., an amount sufficient to reduce the size of a tumor.

Synergy or Synergistic Effect regarding an effect produced by two or more individual components has the biomedical art-recognized meaning of a phenomenon in which the total effect produced by these components, when utilized in combination, is greater than the sum of the individual effects of each component acting alone.

T Cell Cytoxicity has the biomedical art-recognized meaning and includes any immune response that is mediated by CD8⁺ T cell activation. Exemplary immune responses include cytokine production, CD8⁺ T cell proliferation, granzyme or perforin production, and clearance of an infectious agent.

T Cell has the biomedical art-recognized meaning of a CD4+ T cell or a CD8+ T cell. The term T cell encompasses TH1 cells, TH2 cells and TH17 cells.

T Cell Membrane Protein-3 (TIM3) is an inhibitory receptor involved in the inhibition of lymphocyte activity by inhibition of TH1 cells responses. Its ligand is galectin 9, which is upregulated in various types of cancers.

TIM3 as used herein includes human TIM3 (hTIM3), variants, isoforms, and species homologs of hTIM3, and analogs having at least one common epitope. The complete hTIM3 sequence can be found under GenBank Accession No. Q8TDQo.

T Cell Receptor (TCR) has the biomedical art-recognized meaning of a complex of integral membrane proteins that participate in the activation of T-cells in response to an antigen.

Soluble T Cell Receptor (sTCR) has the biomedical art-recognized meaning of soluble versions of the α/β TCR.

Transmission Electron Microscopy (TEM) has the biomedical art-recognized meaning of a technique of imaging the internal structure of solids using a beam of high-energy electrons transmitted through the solid.

Therapeutically Effective Amount is an amount that is effective to ameliorate a symptom of a disease. A therapeutically effective amount can be a prophylactically effective amount as prophylaxis can be considered therapy.

TM in this specification means the HERV-K transmembrane protein.

Triple-Negative Breast Cancer (TNBC) has the biomedical art-recognized meaning.

Transgenic Non-Human Animal has the biomedical art-recognized meaning of a non-human animal having a genome comprising one or more human heavy and/or light chain transgenes or transchromosomes (either integrated or non-integrated into the animal's natural genomic DNA) and which can express fully human antibodies. For example, a transgenic mouse can have a human light chain transgene and either a human heavy chain transgene or human heavy chain transchromosome, such that the mouse produces human anti-HERV-K antibodies when immunized with HERV-K antigen and/or cells expressing HERV-K. The human heavy chain transgene can be integrated into the chromosomal DNA of the mouse, as is the case for transgenic mice, for instance HuMAb mice or the human heavy chain transgene can be maintained extrachromosomally, as is the case for transchromosomal KM mice as described in International Pat. Publ. WO 2002/43478. Such transgenic and transchromosomal mice can produce multiple isotypes of human mAbs to a given antigen (such as IgG, IgA, IgM, IgD, or IgE) by undergoing V-D-J recombination and isotype switching. Transgenic, nonhuman animal can also be used for production of antibodies against a specific antigen by introducing genes encoding such specific antibody, for example by operatively linking the genes to a gene which is expressed in the milk of the animal.

Treatment has the biomedical art-recognized meaning of the administration of an effective amount of a therapeutically active compound of the invention with the purpose of easing, ameliorating, arresting, or eradicating (curing) symptoms or disease states.

Tumor-infiltrating lymphocytes (TILs) has the biomedical art-recognized meaning of lymphoid cells (T cells) that infiltrate solid tumors and appear naturally reactive to autologous tumor antigens.

Vector has the biomedical art-recognized meaning of a nucleic acid molecule capable of transporting another nucleic acid to which it was linked. One type of vector is a plasmid, which has the biomedical art-recognized meaning of a circular double-stranded DNA loop into which additional DNA segments can be ligated. Another type of vector is a viral vector, wherein additional DNA segments can be ligated into the viral genome. Some vectors are capable of autonomous replication in a host cell into which they are introduced (for instance bacterial vectors having a bacterial origin of replication and episomal mammalian vectors). Other vectors (such as non-episomal mammalian vectors) can be integrated into the genome of a host cell upon introduction into the host cell, and thereby are replicated along with the host genome. Moreover, some vectors can direct the expression of genes to which they are operatively linked. Such vectors are referred to herein as recombinant expression vectors (or simply, expression vectors). Expression vectors useful in recombinant DNA techniques are often in the form of plasmids. In this specification, the terms plasmid and vector are used interchangeably as the plasmid is the most commonly used form of vector. However, the invention is intended to include such other forms of expression vectors, such as viral vectors (such as replication-defective retroviruses, adenoviruses, and adeno-associated viruses), which serve equivalent functions.

Unless otherwise defined, scientific and technical terms used with this application shall have the meanings commonly understood by persons having ordinary skill in the biomedical art. This invention is not limited to the particular methodology, protocols, reagents, etc., described herein and can vary.

The disclosure described herein does not concern a process for cloning humans, methods for modifying the germ line genetic identity of humans, uses of human embryos for industrial or commercial purposes, or procedures for modifying the genetic identity of animals likely to cause them suffering with no substantial medical benefit to man or animal and animals resulting from such processes.

### Cancer Therapeutic Antibodies

The development of cancer therapeutic antibodies, such as Herceptin® (trastuzumab), Avastin® (bevacizumab), Erbitux® (cetuximab), and others saved many tens of thousands of lives worldwide. In particular, the treatment of HER2-positive metastatic breast or ovarian cancer using trastuzumab has dramatically changed patient outcomes. Antibody therapeutics offer distinct advantages relative to small molecule drugs, namely: (i) defined mechanisms of action; (ii) higher specificity and fewer-off target effects; and (iii) predictable safety and toxicological profiles. As extensive studies with anti-Her2 and anti-EGFR monoclonals attest, only a few antibodies out of many thousands identified based on their ability to bind to their molecular target with high affinity exhibit properties required for clinically effective cancer cell killing. The efficacy of therapeutic antibodies results primarily from their ability to elicit potent tumor cytotoxicity either via direct induction of apoptosis in target cells or through effector-mediated functions like antibody dependent cell-mediated cytotoxicity (ADCC) and complement dependent cytotoxicity (CDC).

The major methodologies for antibody isolation are: (i) in vitro screening of libraries from immunized animals or from synthetic libraries using phage or microbial display, and (ii) isolation of antibodies following B cell immortalization or cloning. An advance that accelerated the approval of therapeutic mAbs was the generation of humanized antibodies by the complementary-determining region (CDR) grafting technique. In CDR grafting, non-human antibody CDR sequences are transplanted into a human framework sequence to maintain target specificity.

### Humanized Antibody and Antibody-Drug Conjugate (ADC) Pharmaceutical Compositions

Cancer cells overexpressing HERV-K may be particularly good targets for the anti-HERV-K humanized antibodies and ADCs of the invention, since more antibodies may be bound per cell. Thus, in a sixty-eighth embodiment, a cancer patient to be treated with anti-HERV-K humanized antibodies or ADCs of the invention is a patient, e.g., a breast cancer, ovarian cancer, pancreatic cancer, lung cancer or colorectal cancer patient who was diagnosed to have overexpression of HERV-K in their tumor cells.

Upon purifying anti-HERV-K humanized antibodies or ADCs they may be formulated into pharmaceutical compositions using well known pharmaceutical carriers or excipients.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed. (Mack Publishing Co., Easton, Pa., 1995).

The pharmaceutically acceptable carriers or diluents as well as any other known adjuvants and excipients should be suitable for the humanized antibodies or ADCs of the invention and the chosen mode of administration. Suitability for carriers and other components of pharmaceutical compositions is determined based on the lack of significant negative impact on the desired biological properties of the chosen compound or pharmaceutical composition of the invention (e.g., less than a substantial impact (10% or less relative inhibition, 5% or less relative inhibition, etc.)) on antigen binding.

A pharmaceutical composition of the invention may also include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The actual dosage levels of the humanized antibodies or ADCs in the pharmaceutical compositions of the invention may be varied to obtain an amount of the humanized antibodies or ADCs which is effective to achieve the desired therapeutic response for a particular patient, composition, and mode of administration, without being toxic to the patient. The selected dosage level will depend upon a variety of pharmacokinetic factors including the activity of the particular compositions of the invention used, the route of administration, the time of administration, the rate of excretion of the particular compound being used, the duration of the treatment, other drugs, compounds and/or materials used in combination with the particular compositions used, the age, sex, weight, condition, general health and prior medical history of the patient being treated, and like factors well known in the medical arts.

The pharmaceutical composition may be administered by any suitable route and mode. Suitable routes of administering the humanized antibodies or ADCs of the invention are well known in the art and may be selected by those of ordinary skill in the molecular biological art.

In a sixty-ninth embodiment, the pharmaceutical composition of the invention is administered parenterally.

The phrases parenteral administration and administered parenterally as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In a seventieth embodiment, the pharmaceutical composition is administered by intravenous or subcutaneous injection or infusion.

Pharmaceutically acceptable carriers include any and all suitable solvents, dispersion media, coatings, antibacterial and antifungal agents, isotonicity agents, antioxidants and absorption delaying agents, and the like that are physiologically compatible with humanized antibodies or ADCs of the invention.

Examples of suitable aqueous and nonaqueous carriers which may be used in the pharmaceutical compositions of the invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except as far as any conventional media or agent is incompatible with the anti-HERV-K humanized antibodies or ADCs of the invention, use thereof in the pharmaceutical compositions of the invention is contemplated.

Proper fluidity may be maintained, for example, using coating materials, such as lecithin, by the maintenance of the required particle size for dispersions, and using surfactants.

The pharmaceutical compositions of the invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the invention may also comprise isotonicity agents, such as sugars, polyalcohols, such as mannitol, sorbitol, glycerol, or sodium chloride in the compositions.

The pharmaceutical compositions of the invention may also contain one or more adjuvants appropriate for the chosen route of administration such as preservatives, wetting agents, emulsifying agents, dispersing agents, preservatives, or buffers, which may enhance the shelf life or effectiveness of the pharmaceutical composition. The anti-HERV-K humanized antibodies or ADCs of the invention may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the molecular biological art. Methods for the preparation of such formulations are generally known to those skilled in the molecular biological art. See e.g., Sustained and Controlled Release Drug Delivery Systems, J. R. Robinson, ed. (Marcel Dekker, Inc., New York, 1978).

In a seventy-first embodiment, the anti-HERV-K humanized antibodies or ADCs of the invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except as far as any conventional media or agent is incompatible with the active compound, use thereof in the pharmaceutical compositions of the invention is contemplated. Supplementary active compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, using a coating such as lecithin, by the maintenance of the required particle size for dispersion and using surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the anti-HERV-K humanized antibodies or ADCs in the required amount in an appropriate solvent with one or a combination of ingredients, e.g., as enumerated above, as required, followed by sterilization microfiltration. Generally, dispersions are prepared by incorporating the anti-HERV-K humanized antibodies or ADCs into a sterile vehicle that contains a basic dispersion medium and the required other ingredients e.g., from those enumerated above. For sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the anti-HERV-K humanized antibodies or ADCs in the required amount in an appropriate solvent with one or a combination of ingredients enumerated above, as required, followed by sterilization microfiltration. Generally, dispersions are prepared by incorporating the anti-HERV-K humanized antibodies or ADCs into a sterile vehicle that contains a basic dispersion medium and the required other ingredients from those enumerated above. For sterile powders for the preparation of sterile injectable solutions, examples of methods of preparation are vacuum drying and freeze-drying (lyophilization) that yield a powder of the anti-HERV-K humanized antibodies or ADCs plus any additional desired ingredient from a previously sterile-filtered solution thereof.

The pharmaceutical composition of the invention may contain one anti-HERV-K humanized antibodies or ADCs of the invention or a combination of anti-HERV-K humanized antibodies or ADCs of the invention.

The efficient dosages and the dosage regimens for the anti-HERV-K humanized antibodies or ADCs depend on the disease or condition to be treated and may be determined by the persons skilled in the molecular biological art. An exemplary, non-limiting range for a therapeutically effective amount of a compound of the invention is about 2-12 mg/kg. An exemplary, non-limiting range for a therapeutically effective amount of an anti-HERV-K humanized antibodies or ADCs of the invention is about 0.1-5 mg/kg.

A physician having ordinary skill in the molecular biological art may readily determine and prescribe the effective amount of the pharmaceutical composition required. For example, the physician could start doses of the anti-HERV-K humanized antibodies or ADCs used in the pharmaceutical composition at levels lower than that required to achieve the desired therapeutic effect and gradually increase the dosage until the desired effect is achieved. A suitable daily dose of a composition of the invention will be that amount of the compound which is the lowest dose effective to produce a therapeutic effect. Such an effective dose will generally depend upon the factors described above. Administration can be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for an anti-HERV-K humanized antibodies or ADCs of the invention to be administered alone, it is preferable to administer the anti-HERV-K humanized antibodies or ADCs as a pharmaceutical composition as described above.

In a seventy-second embodiment, the anti-HERV-K humanized antibodies or ADCs may be administered by infusion in a weekly dosage of from 10 to 1500 mg/m², such as from 30 to 1500 mg/m², or such as from 50 to 1000 mg/m², or such as from 10 to 500 mg/m², or such as from 100 to 300 mg/m². Such administration may be repeated, e.g., one time to eight times. The administration may be performed by continuous infusion over a period of from two hours to twenty-four hours.

In a seventy-third embodiment, the anti-HERV-K humanized antibodies or ADCs may be administered by infusion every third week in a dosage of from 30 to 1500 mg/m², such as from 50 to 1000 mg/m² or 100 to 300 mg/m². Such administration may be repeated, e.g., one time to eight times. The administration may be performed by continuous infusion over a period of from two hours to twenty-four hours.

In a seventy-fourth embodiment, the anti-HERV-K humanized antibodies or ADCs may be administered by slow continuous infusion over a prolonged period, such as more than 24 hours, to reduce toxic side effects.

In a seventy-fifth embodiment the anti-HERV-K humanized antibodies or ADCs may be administered in a weekly dosage of 50 mg to 2000 mg, most preferably from about 2 mg/kg to about 12 mg/kg, for up to sixteen times or more, preferably at least fifty doses (where the antibody is administered every week). The administration may be performed by continuous infusion over a period from two hours to twenty-four hours. Preferred dosage regimens include 4 mg/kg antibody administered as a 2-hour infusion, followed by a weekly maintenance dose of 2 mg/kg antibody which can be administered as a 30-minute infusion if the initial loading dose is well tolerated. Such regimen may be repeated one or more times as necessary, for example, after six months or 12 months. The dosage may be determined or adjusted by measuring the amount of anti-HERV-K humanized antibodies or ADCs of the invention in the blood upon administration, by for instance taking out a biological sample and using anti-idiotypic antibodies which target the antigen binding region of the anti-HERV-K humanized antibodies or ADCs of the invention.

In a seventy-sixth embodiment, the anti-HERV-K humanized antibodies or ADCs may be administered by maintenance therapy, such as, e.g., once a week for a period of 6 months or more.

In a seventy-seventh embodiment, the ADC may be administered by a regimen including one infusion of an ADC of the invention followed by an infusion of an anti-HERV-K antibody of the invention, such as antibody 6H5hum.

### Bispecific T Cell Engagers (BiTEs)

In a seventy-eighth embodiment, provided herein is a method of treating a HERV-K-positive cancer in a subject in need thereof, comprising administering to the subject a therapeutically effective amount of a bispecific antibody comprising two different antigen-binding regions, one which has a binding specificity for CD3 or CD8 and one which has a binding specificity for HERV-K.

In a seventy-ninth embodiment, the invention relates to a bispecific antibody comprising a first single chain human variable region which binds to HERV-K, in series with a second single chain human variable region which binds to T cell activation ligand CD3 or CD8. T the first and second single chain human variable regions are in amino to carboxy order, wherein a linker sequence intervenes between each of said segments, and wherein a spacer polypeptide links the first and second single chain variable regions.

In an eightieth embodiment of the method, the administering is intravenous or intraperitoneal.

In an eighty-first embodiment of the method, the bispecific binding molecule is not bound to a T cell during said administering step.

In an eighty-second embodiment of a method described herein, the method further comprises administering T cells to the subject. In an eighty-third embodiment, the T cells are bound to molecules identical to said bispecific binding molecule.

In an eighty-fourth embodiment, provided herein is a pharmaceutical composition comprising a therapeutically effective amount of the bispecific binding molecule, a pharmaceutically acceptable carrier, and T cells. In an eighty-fifth embodiment, the T cells are bound to the bispecific binding molecule. In an eighty-sixth embodiment, the binding of the T cells to the bispecific binding molecule is noncovalently. In an eighty-seventh embodiment, the administering is performed in combination with T cell infusion to a subject for treatment of a HERV-K-positive cancer. In an eighty-eighth embodiment, the administering is performed after treating the patient with T cell infusion. In an eighty-ninth embodiment, the T cells are autologous to the subject to whom they are administered. In a forty-sixth embodiment, the T cells are allogeneic to the subject to whom they are administered. In a ninetieth embodiment, the T cells are human T cells.

In a ninety-first embodiment of a method described herein, the subject is a human.

In a ninety-second embodiment of the method, the bispecific binding molecule is contained in a pharmaceutical composition, which pharmaceutical composition further comprises a pharmaceutically acceptable carrier.

In a ninety-third embodiment of the bispecific binding molecule, the bispecific binding molecule does not bind an Fc receptor in its soluble or cell-bound form. In a ninety-fourth embodiment of the bispecific binding molecule, the heavy chain was mutated to destroy an N-linked glycosylation site. In a ninety-fifth embodiment of the bispecific binding molecule, the heavy chain has an amino acid substitution to replace an asparagine that is an N-linked glycosylation site, with an amino acid that does not function as a glycosylation site. In a ninety-sixth embodiment of the bispecific binding molecule, the heavy chain was mutated to destroy a C1q binding site. In a ninety-seventh embodiment, the bispecific binding molecule does not activate complement.

In a ninety-eighth embodiment of the bispecific binding molecule, the HERV-K-positive cancer is breast cancer, ovarian cancer, prostate cancer, pancreatic cancer, melanoma, colorectal cancer, small cell lung cancer, non-small cell lung cancer or any other neoplastic tissue that expresses HERV-K. In a ninety-ninth embodiment, the HERV-K-positive cancer is a primary tumor or a metastatic tumor, e.g., brain, bone, or lung metastases.

### DNA-Encoded Bi-Specific T Cell Engagers (DBITE)

Specific antibody therapy, including mAbs and bispecific T cell engagers (BiTEs), are important tools for cancer immunotherapy. BiTEs are a class of artificial bi-specific monoclonal antibodies that has the potential to transform the immunotherapy landscape for cancer. BiTEs direct a host's immune system, more specifically the T cells' cytotoxic activity, against cancer cells. BiTEs have two binding domains. One domain binds to the targeted tumor (like HERV-K-expressing cells) while the other engages the immune system by binding directly to molecules on T cells. This double-binding activity drives T cell activation directly at the tumor resulting in a killing function and tumor destruction. DBiTEs share many advantages of bi-specific monoclonal antibodies. Both are composed of engineered DNA sequences which encode two antibody fragments. The patient's own cells become the factory to manufacture functional BiTES encoded by the delivered DBiTE sequences. Delivery of BiTEs and permitting combinations of DBiTEs to be administered at one time as a multi-pronged approach to treat resistant cancer. Synthetic DNA designs for BiTE-like molecules include engineering and encoding them in optimized synthetic plasmid DNA cassettes. DBiTEs are then injected locally into the muscle and muscle cells convert the genetic instructions into protein to allow for direct in vivo launching of the molecule directly into the bloodstream to the seek and destroy tumors. See, Perales-Puchalt et al., JCI Insight, 4 (8), e126086 (Apr. 18, 2019). In preclinical studies, DBiTEs demonstrated a unique profile compared to conventional BiTEs, overcoming some of the technical challenges associated with production. For further information, see also, PCT Patent Publications WO 2016/054153 (The Wistar Institute of Anatomy and Biology) and WO 2018/041827 (Psioxus Therapeutics Limited).

### HERV-K CAR-T Therapies

Many formulations of CARs specific for target antigens have been developed. See e.g., International Pat. Publ. WO 2014/186469 (Board of Regents, the University of Texas System). This specification provides a method of generating chimeric antigen receptor (CAR)-modified T cells with long-lived in vivo potential for the purpose of treating, for example, leukemia patients exhibiting minimal residual disease (MRD). In aggregate, this method describes how soluble molecules such as cytokines can be fused to the cell surface to augment therapeutic potential. The core of this method relies on co-modifying CAR T cells with a human cytokine mutein of interleukin-15 (IL-15), henceforth referred to as mIL15. The mIL15 fusion protein is comprised of codon-optimized cDNA sequence of IL-15 fused to the full length IL15 receptor alpha via a flexible serine-glycine linker. This IL-15 mutein was designed in such a fashion so as to: (i) restrict the mIL15 expression to the surface of the CAR+ T cells to limit diffusion of the cytokine to non-target in vivo environments, thereby potentially improving its safety profile as exogenous soluble cytokine administration has led to toxicities; and (ii) present IL-15 in the context of IL-15Ra to mimic physiologically relevant and qualitative signaling as well as stabilization and recycling of the IL15/IL15Ra complex for a longer cytokine half-life. T cells expressing mIL15 are capable of continued supportive cytokine signaling, which is critical to their survival post-infusion. The mIL15+CAR+ T cells generated by non-viral Sleeping Beauty System genetic modification and subsequent ex vivo expansion on a clinically applicable platform yielded a T cell infusion product with enhanced persistence after infusion in murine models with high, low, or no tumor burden. Moreover, the mIL 15 CAR+ T cells also demonstrated improved anti-tumor efficacy in both the high and low tumor burden models. A hu6H5 scFv was used to generate a K-CAR in a lentiviral vector.

### T Cell Receptors (TCRs)

T cell receptor (abbreviated TCR) has the biotechnological art-recognized meaning of a heterodimeric molecule comprising an alpha polypeptide chain (alpha chain) and a beta polypeptide chain (beta chain), wherein the heterodimeric receptor is capable of binding to a peptide antigen presented by an HLA molecule. The term also includes so-called gamma/delta TCRs. This description also relates to nucleic acids, vectors, and host cells for expressing TCRs and peptides of this description; and methods of using the same. The peptides of the invention were shown to be capable of stimulating T cell responses and/or are over-presented and thus can be used to produce antibodies and/or TCRs, such as soluble TCRs (sTCRs), according to the invention. The peptides when complexed with the respective Major Histocompatibility Complex can be used to produce antibodies and/or TCRs, in particular sTCRs, according to the invention, as well. Respective methods are well known to the person of skill in the molecular biological art and can be found in the molecular biological literature as well. Thus, the peptides of the invention are useful for generating an immune response in a patient by which tumor cells can be destroyed. An immune response in a patient can be induced by direct administration of the described peptides or suitable precursor substances (e.g., elongated peptides, proteins, or nucleic acids encoding these peptides) to the patient, ideally in combination with an agent enhancing the immunogenicity (i.e., an adjuvant). The immune response originating from such a therapeutic vaccination can be expected to be highly specific against tumor cells because the target peptides of the invention are not presented on normal tissues in comparable copy numbers, preventing the risk of undesired autoimmune reactions against normal cells in the patient. In this context, particularly preferred are the peptides of the invention selected from the group consisting of sequences from Example 5.

In a hundredth embodiment, the description provides a method of producing a TCR as described herein, the method comprising culturing a host cell capable of expressing the TCR under conditions suitable to promote expression of the TCR.

In a hundred-and-first embodiment, the description provides methods disclosed in this specification, wherein the antigen is loaded onto class I or II Major Histocompatibility Complex molecules expressed on the surface of a suitable antigen-presenting cell or artificial antigen-presenting cell by contacting enough of the antigen with an antigen-presenting cell or the antigen is loaded onto class I or II MHC tetramers by tetramerizing the antigen/class I or II Major Histocompatibility Complex monomers.

The alpha and beta chains of alpha/beta TCR's, and the gamma and delta chains of gamma/delta TCRs, are generally regarded as each having two domains, namely variable and constant domains. The variable domain consists of a concatenation of variable region (V) and joining region (J). The variable domain can also include a leader region (L). Beta and delta chains can also include a diversity region (D). The alpha and beta constant domains can also include C-terminal transmembrane (TM) domains that anchor the alpha and beta chains to the cell membrane.

TCRs described in this specification can comprise a detectable label selected from the group consisting of a radionuclide, a fluorophore and biotin. TCRs of this description can be conjugated to a therapeutically active agent, such as a radionuclide, a chemotherapeutic agent, or a toxin.

In a hundred-and-second embodiment, a TCR of this description having at least one mutation in the alpha chain and/or having at least one mutation in the beta chain has modified glycosylation compared to the unmutated TCR.

In a hundred-and-third embodiment, a TCR comprising at least one mutation in the TCR alpha chain and/or TCR beta chain has a binding affinity for, and/or a binding half-life for, a peptide-HLA molecule complex, which is at least double that of a TCR comprising the unmutated TCR alpha chain and/or unmutated TCR beta chain. Affinity-enhancement of tumor-specific TCRs, and its exploitation, relies on the existence of a window for optimal TCR affinities. The existence of such a window is based on observations that TCRs specific for HLA-A2-restricted pathogens have KD values that are generally about 10-fold lower when compared to TCRs specific for HLA-A2-restricted tumor-associated self-antigens. Although tumor antigens have the potential to be immunogenic because tumors arise from the individual's own cells only mutated proteins or proteins with altered translational processing will be seen as foreign by the immune system. Antigens that are up-regulated or overexpressed (so called self-antigens) will not necessarily induce a functional immune response against the tumor: T cells expressing TCRs that are highly reactive to these antigens will have been negatively selected within the thymus in a process known as central tolerance, meaning that only T cells with low-affinity TCRs for self-antigens remain. Therefore, affinity of TCRs or variants of this description to peptides can be enhanced by methods well known in the art.

This invention further relates to a method of identifying and isolating a TCR according to this description, said method comprising incubating PBMCs from HLA-A*02-negative healthy donors with A2/peptide monomers, incubating the PBMCs with tetramer-phycoerythrin (PE) and isolating the high avidity T cells by fluorescence activated cell sorting (FACS)-Calibur analysis.

This invention further relates to a method of identifying and isolating a TCR according to this description, said method comprising obtaining a transgenic mouse with the entire human TCRap gene loci (1.1 Mb and 0.7 Mb), whose T cells express a diverse human TCR repertoire that compensates for mouse TCR deficiency, immunizing the mouse with a peptide, incubating PBMCs obtained from the transgenic mice with tetramer-phycoerythrin (PE), and isolating the high avidity T cells by fluorescence activated cell sorting (FACS)-Calibur analysis.

In a hundred-and-fourth embodiment, to obtain T cells expressing TCRs of this description, nucleic acids encoding TCR-alpha and/or TCR-beta chains of this description are cloned into expression vectors, such as gamma retrovirus or lentivirus. The recombinant viruses are generated and then tested for functionality, such as antigen specificity and functional avidity. An aliquot of the final product is then used to transduce the target T cell population (generally purified from patient PBMCs), which is expanded before infusion into the patient.

In a hundred-and-fifth embodiment, to obtain T cells expressing TCRs of this description, TCR RNAs are synthesized by techniques known in the art, e.g., in vitro transcription systems. The in vitro-synthesized TCR RNAs are then introduced into primary CD8+ T cells obtained from healthy donors by electroporation to re-express tumor specific TCR-alpha and/or TCR-beta chains.

To increase the expression, nucleic acids encoding TCRs of this description can be operably linked to strong promoters, such as retroviral long terminal repeats (LTRs), cytomegalovirus (CMV), murine stem cell virus (MSCV) U3, phosphoglycerate kinase (PGK), beta-actin, ubiquitin, and a simian virus 40 (SV40)/CD43 composite promoter, elongation factor (EF)-1a and the spleen focus-forming virus (SFFV) promoter. In a hundred-and-sixth embodiment, the promoter is heterologous to the nucleic acid being expressed.

In addition to strong promoters, TCR expression cassettes of this description can contain additional elements that can enhance transgene expression, including a central polypurine tract (cPPT), which promotes the nuclear translocation of lentiviral constructs, and the woodchuck hepatitis virus posttranscriptional regulatory element (wPRE), which increases the level of transgene expression by increasing RNA stability.

The alpha and beta chains of a TCR of the invention can be encoded by nucleic acids located in separate vectors or can be encoded by polynucleotides located in the same vector.

Achieving high-level TCR surface expression requires that both the TCR-alpha and TCR-beta chains of the introduced TCR be transcribed at high levels. To do so, the TCR-alpha and TCR-beta chains of this invention can be cloned into bi-cistronic constructs in a single vector, which was shown to be capable of over-coming this obstacle. The use of a viral intraribosomal entry site (IRES) between the TCR-alpha and TCR-beta chains results in the coordinated expression of both chains, because the TCR-alpha and TCR-beta chains are generated from a single transcript that is broken into two proteins during translation, ensuring that an equal molar ratio of TCR-alpha and TCR-beta chains are produced.

Nucleic acids encoding TCRs of this description can be codon optimized to increase expression from a host cell. Redundancy in the genetic code allows some amino acids to be encoded by more than one codon, but some codons are less optimal than others because of the relative availability of matching tRNAs as well as other factors. Modifying the TCR-alpha and TCR-beta gene sequences such that each amino acid is encoded by the optimal codon for mammalian gene expression, as well as eliminating mRNA instability motifs or cryptic splice sites, was shown to significantly enhance TCR-alpha and TCR-beta gene expression.

Mispairing between the introduced and endogenous TCR chains can result in the acquisition of specificities that pose a significant risk for autoimmunity. For example, the formation of mixed TCR dimers can reduce the number of CD3 molecules available to form properly paired TCR complexes, and therefore can significantly decrease the functional avidity of the cells expressing the introduced TCR.

To reduce mispairing, the C-terminus domain of the introduced TCR chains of this description can be modified to promote interchain affinity, while decreasing the ability of the introduced chains to pair with the endogenous TCR. These strategies can include replacing the human TCR-alpha and TCR-beta C-terminus domains with their murine counterparts (murinized C-terminus domain); generating a second interchain disulfide bond in the C-terminus domain by introducing a second cysteine residue into both the TCR-alpha and TCR-beta chains of the introduced TCR (cysteine modification); swapping interacting residues in the TCR-alpha and TCR-beta chain C-terminus domains (knob-in-hole); and fusing the variable domains of the TCR-alpha and TCR-beta chains directly to CD3 (CD3 fusion).

In a hundred-and-seventh embodiment, a host cell is engineered to express a TCR of this description. In a hundred-and-eighth embodiment, the host cell is a human T cell or T cell progenitor. In a hundred-and-ninth embodiment, the T cell or T cell progenitor is obtained from a cancer patient. In a hundred-and-tenth embodiment, the T cell or T cell progenitor is obtained from a healthy donor. Host cells of this description can be allogeneic or autologous with respect to a patient to be treated. In a hundred-and-eleventh embodiment, the host is a gamma/delta T cell transformed to express an alpha/beta TCR.

T cell antigen coupler (TAC), a TCR-dependent, Major Histocompatibility Complex-independent therapy, has three components: (1) an antigen-binding domain, (2) a TCR-recruitment domain, and (3) a co-receptor domain (hinge, transmembrane, and cytosolic regions). TAC, a chimeric receptor that co-opts the endogenous TCR, induces more efficient anti-tumor responses and reduced toxicity when compared with past-generation CARs. The TAC receptor is designed to trigger aggregation of the native TCR following binding of tumor antigens by co-opting the native TCR via the CD3 binding domain.

In a solid tumor model, TAC-T cells outperform CD28-based CAR-T cells with increased anti-tumor efficacy, reduced toxicity, and faster tumor infiltration. Intratumoral TAC-T cells are enriched for Ki-67⁺ CD8⁺ T cells, demonstrating local expansion. TAC-engineered T cells induce robust and antigen-specific cytokine production and cytotoxicity in vitro, and strong anti-tumor activity in a variety of xenograft models including solid and liquid tumors.

Upon purifying anti-HERV-K T-cell, drug, and vaccine therapeutics they may be formulated into pharmaceutical compositions using well known pharmaceutical carriers or excipients.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed. (Mack Publishing Co., Easton, Pa., 1995).

The pharmaceutically acceptable carriers or diluents as well as any other known adjuvants and excipients should be suitable for the T-cell, drug and vaccine therapeutics of the invention and the chosen mode of administration. Suitability for carriers and other components of pharmaceutical compositions is determined based on the lack of significant negative impact on the desired biological properties of the chosen compound or pharmaceutical composition of the invention (e.g., less than a substantial impact (10% or less relative inhibition, 5% or less relative inhibition, etc.)) on antigen binding.

A pharmaceutical composition of the invention may also include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The actual dosage levels of the T-cell, drug, and vaccine therapeutics in the pharmaceutical compositions of the invention may be varied to obtain an amount of the T-cell, drug and vaccine therapeutics which are effective to achieve the desired therapeutic response for a particular patient, composition, and mode of administration, without being toxic to the patient. The selected dosage level will depend upon a variety of pharmacokinetic factors including the activity of the particular compositions of the invention used, the route of administration, the time of administration, the rate of excretion of the particular compound being used, the duration of the treatment, other drugs, compounds and/or materials used in combination with the particular compositions used, the age, sex, weight, condition, general health and prior medical history of the patient being treated, and like factors well known in the medical arts.

The pharmaceutical composition may be administered by any suitable route and mode. Suitable routes of administering the T-cell, drug and vaccine therapeutics of the invention are well known in the biomedical art and may be selected by those of ordinary skill in the biomedical art.

In a hundred-and-twelfth embodiment, the pharmaceutical composition of the invention is administered parenterally.

The phrases parenteral administration and administered parenterally as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In a hundred-and-thirteenth embodiment, the pharmaceutical composition is administered by intravenous or subcutaneous injection or infusion.

Pharmaceutically acceptable carriers include any and all suitable solvents, dispersion media, coatings, antibacterial and antifungal agents, isotonicity agents, antioxidants and absorption delaying agents, and the like that are physiologically compatible with T-cell, drug, and vaccine therapeutics of the invention.

Examples of suitable aqueous and nonaqueous carriers which may be used in the pharmaceutical compositions of the invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except as far as any conventional media or agent is incompatible with the anti-HERV-K T-cell, drug, and vaccine therapeutics of the invention, use thereof in the pharmaceutical compositions of the invention is contemplated.

Proper fluidity may be maintained, for example, using coating materials, such as lecithin, by the maintenance of the required particle size for dispersions, and using surfactants.

The pharmaceutical compositions of the invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the invention may also comprise isotonicity agents, such as sugars, polyalcohols, such as mannitol, sorbitol, glycerol, or sodium chloride in the compositions.

The pharmaceutical compositions of the invention may also contain one or more adjuvants appropriate for the chosen route of administration such as preservatives, wetting agents, emulsifying agents, dispersing agents, preservatives, or buffers, which may enhance the shelf life or effectiveness of the pharmaceutical composition. The anti-HERV-K T-cell, drug and vaccine therapeutics of the invention may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the molecular biological art. Methods for the preparation of such formulations are generally known to those skilled in the molecular biological art. See e.g., Sustained and Controlled Release Drug Delivery Systems, J. R. Robinson, ed. (Marcel Dekker, Inc., New York, 1978).

In a hundred-and-fourteenth embodiment, the anti-HERV-K T-cell, drug and vaccine therapeutics of the invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except as far as any conventional media or agent is incompatible with the active compound, use thereof in the pharmaceutical compositions of the invention is contemplated. Supplementary active compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, using a coating such as lecithin, by the maintenance of the required particle size for dispersion and using surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the anti-HERV-K T-cell, drug, and vaccine therapeutics in the required amount in an appropriate solvent with one or a combination of ingredients, e.g., as enumerated above, as required, followed by sterilization microfiltration. Generally, dispersions are prepared by incorporating the anti-HERV-K T-cell, drug and vaccine therapeutics into a sterile vehicle that contains a basic dispersion medium and the required other ingredients, e.g., from those enumerated above. For sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the anti-HERV-K T-cell, drug, and vaccine therapeutics in the required amount in an appropriate solvent with one or a combination of ingredients enumerated above, as required, followed by sterilization microfiltration. Generally, dispersions are prepared by incorporating the anti-HERV-K humanized T-cell, drug and vaccine therapeutics into a sterile vehicle that contains a basic dispersion medium and the required other ingredients from those enumerated above. For sterile powders for the preparation of sterile injectable solutions, examples of methods of preparation are vacuum drying and freeze-drying (lyophilization) that yield a powder of the anti-HERV-K T-cell, drug, and vaccine therapeutics plus any additional desired ingredient from a previously sterile-filtered solution thereof.

The pharmaceutical composition of the invention may contain one anti-HERV-K T-cell, drug and vaccine therapeutics of the invention or a combination of anti-HERV-K T-cell, drug and vaccine therapeutics of the invention, and anti-HERV-K humanized antibodies or ADCs.

The efficient dosages and the dosage regimens for the anti-HERV-K T-cell, drug, and vaccine therapeutics depend on the disease or condition to be treated and may be determined by the persons skilled in the molecular biological art. An exemplary, non-limiting dose range for a therapeutically effective amount of HERV-K TCR T cells is about 1×10⁹ to 1×10¹² cells. HERV-K specific cancer vaccines include HERV-K antigen-derived peptides, proteins, DNA, and mRNA, as well as DCs. Exemplary, non-limiting ranges for therapeutically effective amounts of HERV-K cancer vaccines will vary with the type of vaccine used, as listed above (HERV-K antigen-derived peptides, proteins, DNA, mRNA, DCs). An exemplary, non-limiting range for a therapeutically effective amount of an HERV-K DC vaccine is 5×10⁶ to 1×10⁷ cells per vaccine dose. An exemplary, non-limiting range for a therapeutically effective amount of an HERV-K mRNA or DNA vaccine is 50 μg to 500 μg of mRNA or DNA per dose. The mRNA vaccines are dosed at 2-week intervals for 4 cycles.

A physician having ordinary skill in the molecular biological art may readily determine and prescribe the effective amount of the pharmaceutical composition required. For example, the physician could start doses of the anti-HERV-K T-cell, drug and vaccine therapeutics used in the pharmaceutical composition at levels lower than that required to achieve the desired therapeutic effect and gradually increase the dosage until the desired effect is achieved. A suitable daily dose of a composition of the invention will be that amount of the compound which is the lowest dose effective to produce a therapeutic effect. Such an effective dose will generally depend upon the factors described above. Administration can be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for a T-cell, drug, or vaccine therapeutic of the invention to be administered alone, it is preferable to administer the anti-HERV-K T-cell, drug, or vaccine therapeutic as a pharmaceutical composition as described above.

In a hundred-and-fifteenth embodiment, the anti-HERV-K T-cell, drug, and vaccine therapeutics may be administered by slow continuous infusion over a long period, such as more than twenty-four hours, to reduce toxic side effects.

The dosage may be determined or adjusted by measuring the amount of anti-HERV-K T-cell, drug, and vaccine therapeutics of the invention in the blood upon administration, by for instance analyzing a biological sample.

In a hundred-and-sixteenth embodiment, the anti-HERV-K T-cell, drug, and vaccine therapeutics may be administered by maintenance therapy, such as, e.g., once a week for a period of six months or more.

In a hundred-and-seventeenth embodiment, the anti-HERV-K T-cell, drug, and vaccine therapeutics may be administered by a regimen including initial infusion of one therapeutic of the invention followed by a second infusion of an additional anti-HERV-K T-cell, drug, and vaccine therapeutic of the invention.

In a hundred-and-eighteenth embodiment of the method of treating, the method further comprises administering to the subject the chemotherapeutic agents doxorubicin, cyclophosphamide, paclitaxel, docetaxel, and/or carboplatin. In a hundred-and-nineteenth embodiment of the method of treating, the method further comprises administering to the subject radiotherapy. In a hundred-and-twentieth embodiment of the method of treating, the administering is performed in combination with multi-modality anthracycline-based therapy.

### Combination Therapies

The therapies of this specification can be used without modification, relying on the binding of the antibodies or fragments to the surface antigens of HERV-K+ cancer cells in situ to stimulate an immune attack thereon. Alternatively, the aforementioned method can be carried out using the antibodies or binding fragments to which a cytotoxic agent is bound. Binding of the cytotoxic antibodies, or antibody binding fragments, to the tumor cells inhibits the growth of or kills the cells.

Antibodies specific for HERV-K env protein may be used in conjunction with other expressed HERV antigens. This may be particularly useful for immunotherapy and antibody treatments of diseases in which several different HERVs are expressed. For example, HERV-E in prostate, ERV3, HERV-E and HERV-K in ovarian cancer, and ERV3, HERV-H, and HERV-W in other cancers.

HERV-K env protein may serve as a tumor-associated antigen which can be used to elicit T cell and B cell responses. In therapeutic applications, this can be used to reduce immune tolerance in, for example, a cancer patient. For example, HERV-K env protein is expressed on both the cell surface and cytoplasm of breast cancer cells, therefore providing a target for both B cells and T cells, and potentially greatly increasing the effectiveness of HERV-K as a tumor-associated antigen.

Autologous dendritic cells pulsed with HERV-K env protein enables autologous professional antigen presenting cells to process and present one or more HERV-K epitopes in association with host human leukocyte antigen (HLA) molecules. Accordingly, in particular embodiments a therapeutic method of the present invention comprises pulsing autologous DCs with HERV-K env protein to treat a HERV-K+ cancer. DCs pulsed with HERV-K env protein induce T cell responses, enhance granzyme B secretion, induce CTL responses, and increase the secretion of several T helper type 1 and 2 cytokines.

Anti-HERV-K T-cell, drug, and vaccine therapeutics that target the HERV-K env protein may be used in conjunction with other expressed HERV antigens. This may be particularly useful for immunotherapy and antibody treatments of diseases in which several different HERVs are expressed. For example, HERV-E in prostate, ERV3, HERV-E and HERV-K in ovarian cancer, and ERV3, HERV-H, and HERV-W in other cancers.

Cytokines in the common gamma chain receptor family (γC) are important costimulatory molecules for T cells that are critical to lymphoid function, survival, and proliferation. IL-15 possesses several attributes that are desirable for adoptive therapy. IL-15 is a homeostatic cytokine that supports the survival of long-lived memory cytotoxic T cells, promotes the eradication of established tumors via alleviating functional suppression of tumor-resident cells, and inhibits activation-induced cell death (AICD). IL-15 is tissue restricted and only under pathologic conditions is it observed at any level in the serum, or systemically. Unlike other γC cytokines that are secreted into the surrounding milieu, IL-15 is trans-presented by the producing cell to T cells in the context of IL-15 receptor alpha (IL-15Ra). The unique delivery mechanism of this cytokine to T cells and other responding cells: (i) is highly targeted and localized, (ii) increases the stability and half-life of IL-15, and (iii) yields qualitatively different signaling than is achieved by soluble IL-15.

### Pharmaceutical Compositions

This specification is also directed to pharmaceutical compositions comprising a therapy that specifically binds to a HERV-K env protein, together with a pharmaceutically acceptable carrier, excipient, or diluent. Such pharmaceutical compositions may be administered in any suitable manner, including parental, topical, oral, or local (such as aerosol or transdermal) or any combination thereof. Suitable regimens also include an initial administration by intravenous bolus injection followed by repeated doses at one or more intervals.

Pharmaceutical compositions of the compounds of the disclosure are prepared for storage by mixing a peptide ligand containing compound having the desired degree of purity with optional pharmaceutically acceptable carriers, excipients, or stabilizers (Remington's Pharmaceutical Sciences 18th ed., 1990), in the form of lyophilized formulations or aqueous solutions. Acceptable carriers, excipients, or stabilizers are nontoxic to recipients at the dosages and concentrations used.

The compositions herein may also contain more than one active compound as necessary for the particular indication being treated, preferably those with complementary activities that do not adversely affect each other. Alternatively, or in addition, the composition may comprise a cytotoxic agent, cytokine, growth inhibitory agent, or cardioprotectant. Such molecules are suitably present in combination in amounts that are effective for the purpose intended.

Vaccines may comprise one or more such compounds in combination with an immunostimulant, such as an adjuvant or a liposome (into which the compound is incorporated). An immunostimulant may be any substance that enhances or potentiates an immune response (antibody and/or cell-mediated) to an exogenous antigen. Vaccine preparation is generally described in, for example, M. F. Powell and M. J. Newman, eds., Vaccine Design (the subunit and adjuvant approach), (Plenum Press, New York, 1995). Pharmaceutical compositions and vaccines within the scope of the present disclosure may also contain other compounds, which may be biologically active or inactive. For example, one or more immunogenic portions of other tumor-associated antigens may be present, either incorporated into a fusion polypeptide or as a separate compound, within the composition or vaccine. Humoral or cellular immune responses against tumor-associated antigen may provide a non-toxic modality to treat cancer. The presence of these antigens is also associated with both specific CD4⁺ and CD8⁺ T cell responses. The pharmaceutical compositions and vaccines within the scope of the present disclosure may capitalize on these responses to increase their clinical benefit.

A vaccine may contain DNA encoding one or more of the polypeptides as described above, such that the polypeptide is generated in situ. In such vaccines, the DNA may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art.

Any of a variety of immunostimulants may be used in the vaccines of this disclosure. For example, an adjuvant may be included.

Within the vaccines provided herein, the adjuvant composition is preferably designed to induce an immune response predominantly of the Th1 type. High levels of Th1-type cytokines (e.g., IFNγ, TNFα, IL-2 and IL-12) tend to favor the induction of cell mediated immune responses to an administered antigen. In contrast, elevated levels of Th2-type cytokines (e.g., IL-4, IL-5, IL-6, and IL-10) tend to favor the induction of humoral immune responses. Following application of a vaccine as provided herein, a patient will support an immune response that includes Th1-type and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann & Coffman, Ann. Rev. Immunol. 7:145-173 (1989).

The invention is further illustrated by the following EXAMPLES, which are not intended to limit the scope or content of the invention in any way.

### Example 1

### Humanized Antibodies Produced Commercially and in the Inventors' Laboratories

The inventors synthesized and purified humanized antibodies targeting HERV-K tumor antigens. HERV-K envelope surface gene tumor antigens (KSU) were derived from human patient cancer cells, rather sequences in GenBank, which are HERV-K gene sequences from normal humans. These HERV-K sequences from cancer patients were shown by the inventors to contain variants that differentiated them from the normal HERV-K sequence. The inventors' humanized antibodies are specific for the HERV-K target found in human cancer cells. This specificity distinguishes the HERV-K target found in human cancer cells from the HERV-K target present in tissues from normal individuals or patients with non-cancer disorders. This specificity also distinguishes the inventors' humanized antibodies to HERV-K tumor antigens from other antibodies to HERV-K tumor antigens.

The human antibodies targeted the full-length surface protein of the HERV-K envelope gene, rather than a peptide or a small fragment of the gene. Full length envelope surface protein is only expressed in cancer cells obtained from cancer patients.

The scFv sequence from a murine antibody against HERV-K envelope surface fusion protein was submitted to a contract research organization (CRO) to produce humanized antibodies, but the CRO could not generate the light chain of the inventors' humanized antibody targeting HERV-K. A second CRO generated a chimeric antibody, but that antibody could not bind the HERV-K envelope surface fusion protein by ELISA, SPR, or Western blot assay, even though binding of the chimeric antibody and the inventors' non-humanized mouse antibody was readily detected by the inventors using ELISA or Western blot assays.

The inventors then generated three humanized antibodies. Only one of the three humanized antibodies (HUM1, expressed in bacterial cells) bound HERV-K antigen. Expressed HUM2 or HUM3 protein did not bind the recombinant SU protein well, and especially not to the SU proteins produced from breast cancer cells (MDA-MB-231).

The inventors then produced an additional humanized antibody, hu6H5. Hu6H5 was expressed in mammalian cells and its antitumor effects were determined. Both the HUM1 and hu6H5 antibodies bound to the full-length KSU antigen.

The difference between HUM1, HUM2, and HUM3 is shown in TABLE 1 for VH below and TABLE 2 for VL below. HUM1, HUM2, and HUM3 were all generated from the same bacterial expression vector. All have the same CDRs for VH and VL. Hu6H5 was generated from a mammalian vector based on the HUM1 sequence. HUM1, HUM2, and HUM3 all bound recombinant KSU protein. Only HUM1 bound the protein isolated from cancer cells.

This EXAMPLE unexpectedly shows that the medically useful functional property of antibody binding to protein isolated from cancer cells is not a property arising from the structure of VH and VL CDRs.

### Example 2

Comparison of hu6H5 Sequence with Sequences of Other Humanized Anti-HERV-K Antibodies, and with Sequences of Other Antibodies that Target Tumor Antigens

An anti-HERV-K antibody that targets amyotrophic lateral sclerosis (ALS), called GN-mAb-Env_K01, binds to an HERV-K envelope linear peptide with the SLDKHKHKKLQSFYP core sequence. See U.S. Pat. No. 10,723,787.

The hu6H5 antibody binds to the longer, full-length HERV-K envelope SU domain, and not a linear peptide.

In contrast to GN-mAb-Env_K01, the inventors' humanized antibody was specific for HERV-K ENV protein from a cancer cell target. The hu6H5 sequence shows little sequence similarity to the GN-mAb-Env_K01 humanized antibody. The identities are 60% for VH and 55% for VL.

A BLAST search showed that other antibodies that target cancer-relevant antigens have a minimal amount of homology with hu6H5 (6-15 peptides). These include 82% identity with the VH of anti-ErbB2 antibody (based on the crystal structure of the anti-ErbB2 Fab2C4) that targets HER2-positive breast cancer, and 88% identity with the VL of anti-DREG-55 [(anti-DREG-55 (L-selectin) immunoglobulin light chain variable region]. The anti-DREG-55 target, L-selectin, mediates adaptive and innate immunity in cancer.

The inventors also identified human germline sequences near the boundaries for CDRs in their humanized anti-HERV-K antibodies. These sequences include VRQAPGKGLEW (SEQ ID NO.: 47). and LQMNSLRAEDTAVYYC (SEQ ID NO.: 48).

### Example 3

### Binding Affinity of Anti-HERV-K Humanized Antibodies to HERV-K SU Protein, and Antibody Internalization

The inventors determined by ELISA assay that the HUM1 humanized antibody affinity toward the HERV-K envelope surface fusion protein was as effective as the affinity of their m6H5 at antibody concentrations above 0.00625 μg/ml and was more effective than most of inventors' other murine mAbs. The inventors' chimeric humanized anti-HERV-K antibody was shown by immunoblot to bind and their m6H5 mAb to two HERV-K Env surface proteins, ERVK6 and HERV-K envelope surface fusion protein.

To determine antibody internalization of humanized and murine antibodies, cells were incubated with HUM1 or m6H5 at 37° C. for different time intervals (0, 5, 30, and 45 minutes). At each time point, cells were fixed. Half of the cells were permeabilized and half were not permeabilized. Anti-human IgG 488 or anti-mouse IgG 488 was used to detect the percentages of antibody-bound HERV-K env protein that remained on the cell surface in the cells that were not permeabilized. A reduced surface expression of HERV-K positive cells was observed. The internalization of rate was determined in the cells that were permeabilized. The equation for the percentage of internalization at each point was 100−(% label at time 0).

Results demonstrated an increased expression of internalized HERV-K positive cells in cells treated with either HUM1 or murine 6H5 mAb, but HUM1 disappeared from the cell surface more rapidly than 6H5, indicating a more rapid uptake of the inventors' humanized antibody than their murine antibody. This rapid uptake supports the unique capability of HUM1 to deliver a payload more rapidly than m6H5.

### Example 4

Generation of hu6H5 Ab by CDR Grafting

TABLE 1

| [symbol] PROTEIN | | | | | | | | | | |
|---|---|---|---|---|---|---|---|---|---|---|
| SEQUENCE & | | | | | | | | | | |
| ALIGNED | | | | | | | | | | |
| CODONS | QCAG | VGTG | KAAG | LCTG | QCAG | QCAG | STCA | GGGA | PCCT | DGAC |
| CHOTHIA | H1 | H2 | H3 | H4 | H5 | H6 | H7 | H8 | H9 | H10 |
| NUMBERING | | | | | | | | | | |

| CHOTHIA | HFR1 | | | | | | | | | |
|---|---|---|---|---|---|---|---|---|---|---|
| REGIONS | | | | | | | | | | |
| [symbol] PROTEIN | | | | | | | | | | |
| SEQUENCE & | | | | | | | | | | |
| ALIGNED | | | | | | | | | | |
| CODONS | LCTG | VGTG | KAAG | PCCT | GGGG | AGCT | STCA | VGTG | KAAG | IATA |
| CHOTHIA | H11 | H12 | H13 | H14 | H15 | H16 | H17 | H18 | H19 | H20 |
| NUMBERING | | | | | | | | | | |

| CHOTHIA | HFR1 | | | | | | | | | | | |
|---|---|---|---|---|---|---|---|---|---|---|---|---|
| REGIONS | | | | | | | | | | | | |
| [symbol] PROTEIN | | | | | | | | | | | | |
| SEQUENCE & | | | | | | | | | | | | |
| ALIGNED | | | | | | | | | | | | |
| CODONS | STCC | CTGC | KAAG | AGCG | STCT | GGGT | YTAC | STCA | FTTC | TACT | GGGC | YTAC |
| CHOTHIA | H21 | H22 | H23 | H24 | H25 | H26 | H27 | H28 | H29 | H30 | H31 | H32 |
| NUMBERING | | | | | | | | | | | | |

| | |
|---|---|
| CHOTHIA | CDR [symbol] |
| REGIONS | |

| [symbol] PROTEIN | | | | | | | | | | | |
|---|---|---|---|---|---|---|---|---|---|---|---|
| SEQUENCE & | | | | | | | | | | | |
| ALIGNED | | | | | | | | | | | |
| CODONS | YTAC | MATG | HCAC | WTGG | VGTG | KAAG | QCAG | SAGC | HCAT | GGGA | KAAG |
| CHOTHIA | H33 | H34 | H35 | H36 | H37 | H38 | H39 | H40 | H41 | H42 | H43 |
| NUMBERING | | | | | | | | | | | |

| | |
|---|---|
| CHOTHIA | HFR2 |
| REGIONS | |

| [symbol] PROTEIN | | | | | | | | | | | | |
|---|---|---|---|---|---|---|---|---|---|---|---|---|
| SEQUENCE & | | | | | | | | | | | | |
| ALIGNED | | | | | | | | | | | | |
| CODONS | SAGC | LCTT | EGAG | WTGG | IATT | GGGA | RCGT | VGTT | NAAT | PCCT | NAAC | SAGT |
| CHOTHIA | H44 | H45 | H46 | H47 | H48 | H49 | H50 | H51 | H52 | H52A | H53 | H54 |
| NUMBERING | | | | | | | | | | | | |

| | |
|---|---|
| CHOTHIA | CDR [symbol] |
| REGIONS | |

| [symbol] PROTEIN | | | | | | | | | | | |
|---|---|---|---|---|---|---|---|---|---|---|---|
| SEQUENCE & | | | | | | | | | | | |
| ALIGNED | | | | | | | | | | | |
| CODONS | GGGT | GGGT | TACA | SAGC | YTAC | NAAC | QCAG | KAAG | FTTC | KAAG | DGAC |
| CHOTHIA | H55 | H56 | H57 | H58 | H59 | H60 | H61 | H62 | H63 | H64 | H65 |
| NUMBERING | | | | | | | | | | | |

| | | |
|---|---|---|
| CHOTHIA | CDR [symbol] | HFR3 |
| REGIONS | | |

| [symbol] PROTEIN | | | | | | | | | | | | |
|---|---|---|---|---|---|---|---|---|---|---|---|---|
| SEQUENCE & | | | | | | | | | | | | |
| ALIGNED | | | | | | | | | | | | |
| CODONS | KAAG | AGCC | IATA | LTTA | TACT | VGTA | DGAC | KAAG | STCA | STCC | SAGC | TACA |
| CHOTHIA | H66 | H67 | H68 | H69 | H70 | H71 | H72 | H73 | H74 | H75 | H76 | H77 |
| NUMBERING | | | | | | | | | | | | |
| CHOTHIA | | | | | | | | | | | | |
| REGIONS | | | | | | | | | | | | |

| [symbol] PROTEIN | | | | | | | | | | | |
|---|---|---|---|---|---|---|---|---|---|---|---|
| SEQUENCE & | | | | | | | | | | | |
| ALIGNED | | | | | | | | | | | |
| CODONS | AGCC | YTAC | MATG | EGAG | LCTC | RCGC | SAGC | LCTG | TACA | STCT | EGAG |
| CHOTHIA | H78 | H79 | H80 | H81 | H82 | H82A | H82B | H82C | H83 | H84 | H85 |
| NUMBERING | | | | | | | | | | | |
| CHOTHIA | | | | | | | | | | | |
| REGIONS | | | | | | | | | | | |

| [symbol] PROTEIN | | | | | | | | | | | | |
|---|---|---|---|---|---|---|---|---|---|---|---|---|
| SEQUENCE & | | | | | | | | | | | | |
| ALIGNED | | | | | | | | | | | | |
| CODONS | DGAC | STCT | AGCG | VGTC | YTAT | YTAC | CTGT | AGCA | RAGA | STCG | KAAA | GGGT |
| CHOTHIA | H86 | H87 | H88 | H89 | H90 | H91 | H92 | H93 | H94 | H95 | H96 | H97 |
| NUMBERING | | | | | | | | | | | | |

| | |
|---|---|
| CHOTHIA | CDR [symbol] |
| REGIONS | |

| [symbol] PROTEIN | | | | | | | | | | | |
|---|---|---|---|---|---|---|---|---|---|---|---|
| SEQUENCE & | | | | | | | | | | | |
| ALIGNED | | | | | | | | | | | |
| CODONS | NAAC | YTAC | FTTC | YTAT | AGCT | MATG | DGAC | YTAC | WTGG | GGGC | QCAA |
| CHOTHIA | H98 | H99 | H100 | H100A | H100B | H100C | H101 | H102 | H103 | H104 | H105 |
| NUMBERING | | | | | | | | | | | |

| | | |
|---|---|---|
| CHOTHIA | CDR [symbol] | HFR4 |
| REGIONS | | |

| [symbol] PROTEIN | | | | | | | | |
|---|---|---|---|---|---|---|---|---|
| SEQUENCE & | | | | | | | | |
| ALIGNED | | | | | | | | |
| CODONS | GGGG | TACC | TACG | VGTC | TACC | VGTC | STCC | STCA |
| CHOTHIA | H106 | H107 | H108 | H109 | H110 | H111 | H112 | H113 |
| NUMBERING | | | | | | | | |
| CHOTHIA | | | | | | | | |
| REGIONS | | | | | | | | |

Predicted CK2 Phosphorylation site
Predicted PKC Phosphorylation site
□ Insertion [symbol]
indicates data missing or illegible when filed

TABLE 2
Displaying 1-111 of 111 residues

| Query protein | | | | | | | | | | | | | | | | | | | | |
|---|---|---|---|---|---|---|---|---|---|---|---|---|---|---|---|---|---|---|---|---|
| sequence | D | I | E | L | T | Q | S | P | A | S | L | A | V | S | L | G | Q | R | A | T |
| Chothia | L1 | L2 | L3 | L4 | L5 | L6 | L7 | L8 | L9 | L10 | L11 | L12 | L13 | L14 | L15 | L16 | L17 | L18 | L19 | L20 |
| numbering | | | | | | | | | | | | | | | | | | | | |

| | |
|---|---|
| CHOTHIA | LFR1 |
| REGIONS | |

| Query protein | | | | | | | | | | | | |
|---|---|---|---|---|---|---|---|---|---|---|---|---|
| sequence | I | S | C | R | A | S | E | S | V | D | S | H |
| Chothia | L21 | L22 | L23 | L24 | L25 | L26 | L27 | L28 | L29 | L30 | L30A | L30B |
| numbering | | | | | | | | | | | | |

| | |
|---|---|
| CHOTHIA | CDR-L1 |
| REGIONS | |

| Query protein | | | | | | | | | | | |
|---|---|---|---|---|---|---|---|---|---|---|---|
| sequence | G | T | S | F | M | H | W | Y | Q | Q | K |
| Chothia | L30C | L30D | L31 | L32 | L33 | L34 | L35 | L36 | L37 | L38 | L39 |
| numbering | | | | | | | | | | | |

| | | |
|---|---|---|
| CHOTHIA | CDR-L1 | LFR2 |
| REGIONS | | |

| Query protein | | | | | | | | | | | | |
|---|---|---|---|---|---|---|---|---|---|---|---|---|
| sequence | P | G | Q | P | P | K | F | L | I | Y | R | A |
| Chothia | L40 | L41 | L42 | L43 | L44 | L45 | L46 | L47 | L48 | L49 | L50 | L51 |
| numbering | | | | | | | | | | | | |

| | |
|---|---|
| CHOTHIA | CDR-L2 |
| REGIONS | |

| Query protein | | | | | | | | | | | |
|---|---|---|---|---|---|---|---|---|---|---|---|
| sequence | S | N | L | E | S | G | I | P | A | R | F |
| Chothia | L52 | L53 | L54 | L55 | L56 | L57 | L58 | L59 | L60 | L61 | L62 |
| numbering | | | | | | | | | | | |

| | | |
|---|---|---|
| CHOTHIA | CDR-L2 | LFR3 |
| REGIONS | | |

| Query protein | | | | | | | | | | | | |
|---|---|---|---|---|---|---|---|---|---|---|---|---|
| sequence | S | G | S | G | S | R | T | D | F | T | L | T |
| Chothia | L63 | L64 | L65 | L66 | L67 | L68 | L69 | L70 | L71 | L72 | L73 | L74 |
| numbering | | | | | | | | | | | | |
| CHOTHIA | | | | | | | | | | | | |
| REGIONS | | | | | | | | | | | | |

| Query protein | | | | | | | | | | | |
|---|---|---|---|---|---|---|---|---|---|---|---|
| sequence | I | N | P | V | E | T | D | D | V | A | I |
| Chothia | L75 | L76 | L77 | L78 | L79 | L80 | L81 | L82 | L83 | L84 | L85 |
| numbering | | | | | | | | | | | |
| CHOTHIA | | | | | | | | | | | |
| REGIONS | | | | | | | | | | | |

| Query protein | | | | | | | | | | | | |
|---|---|---|---|---|---|---|---|---|---|---|---|---|
| sequence | Y | Y | C | Q | Q | S | N | E | D | P | P | T |
| Chothia | L86 | L87 | L88 | L89 | L90 | L91 | L92 | L93 | L94 | L95 | L96 | L97 |
| numbering | | | | | | | | | | | | |

| | |
|---|---|
| CHOTHIA | CDR-L1 |
| REGIONS | |

| Query protein | | | | | | | | | | |
|---|---|---|---|---|---|---|---|---|---|---|
| sequence | F | G | G | G | T | K | L | E | I | K |
| Chothia | L98 | L99 | L100 | L101 | L102 | L103 | L104 | L105 | L106 | L107 |
| numbering | | | | | | | | | | |

| | |
|---|---|
| CHOTHIA | LFR4 |
| REGIONS | |

Design of Humanized Single Chain Variable Fragment (scFv) Antibody

Antibody numbering scheme and CDR definitions: The antibody-numbering server is part of KabatMan database (http://www.bioinf.org.uk/) and was used to number all antibody sequences of this study according to the enhanced Chothia scheme. In this humanization study, the inventors combined the Enhanced Chothia numbering with the Contact CDR definition of antibody sequence to position the CDRs of antibody light chain and heavy chains at the following locations: H-CDR1 30-35, H-CDR2 47-58 H-CDR3 93-101, L-CDR1 30-36, L-CDR2 46-55, and L-CDR3 89-96.

Selection of the human template: To generate humanized scFv gene, six Complementary determine regions (CDRs) of mouse VH and VL were grafted onto selected human Frameworks (FRs) showing the highest amino acids sequence identify to the humanization of the given antibodies. The human immunoglobulin germline sequences were used as the selected human FRs for mouse FWJ antibody clone. Human immunoglobulin germline sequences showing the highest amino acid sequences similarity in FRs between human and mouse FWJ VH and VL were identified independently using from V-que (http://www.imgt.org/IMGT_vquest) and Ig-BLA (http://www.ncbi.nlm.nih.gov/igblast). The selected heavy chain VHIII and the chain based conserved germlines. The consensus human FRs was designed among selected germline gene for grafting CDRs residues of FWJ. The amino acid sequences in FRs of mouse VH and VL that differed from consensus human FRs were substituted with human residues, while preserving mouse residues at position known as Vernier zone residues and chain packing residues.

TABLE 3
VLs form HUM2, HUM3, and mu6H5 were compared below:
VL CDRs CRASESVDSHGTSFMH---RASNLE--QQSNEDPPT

| | |
|---|---|
| HUM2_VL | DIQMTQSPSSLSASVGDRVTITCRASESVDSHGTSFMHWYQQKPGKAPKFLIYRASNLES |
| HUM3_VL | DIQMTQSPSSLSASVGDRVTISCRASESVDSHGTSFMHWYQQKPGKSPKFLIYRASNLES |
| FWJ_MUVH | DIELTQSPASLAVSLGQRATISCRASESVDSHGTSFMHWYQQKPGQPPKFLIYRASNLES |
| | **::****:**:.*:*:*.**:***********************:.************* |
| HUM2_VL | GIPSRFSGSGSGTDFTLTISSVQPEDFAVYYCQQSNEDPPTFGGTKVEIK |
| HUM3_VL | GIPSRFSGSGSGTDFTLTISSVQPEDFAIYYCQQSNEDPPTFGGTKVEIK |
| FWJ_MUVH | GIPSRFSGSGSRTDFTLTINPVETDDVAIYYCQQSNEDPPTFGGTKVEIK |
| | ***:***.*** *******..::.:*.*:*****************:*** |
| HUM2_VL | (SEQ ID NO: 9) |
| HUM3_VL | (SEQ ID NO: 10) |
| FWJ_MUVH | (SEQ ID NO: 11) |

“*” means that the residues or nucleotides in that column are identical in all sequences in the alignment. “:” means that conserved substitutions have been observed, according to the COLOR table above. “.” means that semi-conserved substitutions are observed, i.e., amino acids having similar shape.

As highlighted in yellow background in the table, the CDRs for both the mouse and the human CDRs are identical, but major differences in the remainder of the VL sequences may account in part for the inability of HUM2 and HUM3 to bind to the human cancer cell HERV-K envelope protein.

Construction of scFv and test of biological activity against Human KV and 231 antigens. The clone of variable heavy chain and light chain of FWJ_1 and FWJ_2 antibody gene were amplified and synthesized. The gene encoding the scFv is VH-linker-VL with a standard 20 amino acid linker (Gly₄Ser)₃GGGAR (SEQ ID NO: 14). The amplified gene was digested with BssHII and NheI restriction enzymes and insert into a pET-based vector (PAB-myc) containing a pelB promotor for controlling periplasmic protein expression (Novagen, Madison, WI, USA) along with 6×histidine tag at the C-termini for purification by metal affinity chromatography and transformed into DH5a bacterial strain. The transformed clones were amplified in LB with ampicillin broth overnight. The plasmid DNAs were prepared and sent for DNA sequencing. The correct sequence of scFv plasmid was transformed into the T7 Shuffle bacterial strain and the transformed bacteria were used for soluble protein production in periplasmic compartment.

FWJ 1 and FWJ 2 scFv Gene and Translated Protein Sequences: The SEQUENCE LISTING delineates the heavy and light chains and linker arm of FWJ_1 and FWJ_2_scFv. In the engineering of the FWJ_1 and FWJ_2_scFv gene two epitope tags were engineered onto the C terminus: (1) a six His tag to facilitate purification of the encoded scFv by nickel affinity chromatography; and (2) a myc tag to facilitate rapid immunochemical recognition of the expressed scFv.

Induction of ScFv proteins in a bacterial host: The FWJ_1 and FWJ_2scFv clone were transformed into T7 shuffle bacterial strain. T7 shuffle cells and was grown in 1.4 L 2×YT plus ampicillin medium at 37° C. until log-phage (OD600=0.5), induced with 0.3 mM IPTG, and allowed to grow at 30° C. for an additional sixteen hours. After induction, the bacteria were harvested by centrifugation at 8000 g for fifteen minutes at 4° C., and the pellets were stored in −20° C. for at least two hours. The frozen pellets were briefly thawed and suspended in 40 ml of lysis buffer (1 mg/ml lysozyme in PBS plus EDTA-free protease inhibitor cocktail (Thermo Scientific, Waltham, MA, USA). The lysis mixture was incubated on ice for an hour, and then 10 mM MgCL₂ and 1 μg/ml DNase I were added, and the mixture was incubated at 25° C. for twenty minutes. The final lysis mixture was centrifuged at 12000 g for twenty minutes and the supernatants were collected. This supernatant was termed the periplasmic extract used for nickel column affinity chromatography.

Western blot analysis using FWJ 1 and FWJ_2_scFv protein: Lysate Ag and KSU protein were used as antigens target in Dot-blot analyses. 2-5 μg Ag proteins as non-reduced conditional and 1 μg purified protein as negative control were loaded onto nitrocellulose membranes. The membrane was blocked using 3% skimmed milk in PBS for 3 h at room temperature. After that, the membrane was incubated with periplasmic extract of FWJ_1 and FWJ_2 scFv proteins overnight at 4° C. The membrane was washed with sodium phosphate buffered saline with 0.05% tween 20 buffer (PBST) 3 times. The washed membrane was incubated with anti-c Myc mouse IgG for 1 h at room temperature to recognize the c-Myc tag on the scFv and identify the position of antigens bound by the scFv. After washing with PBST, the membrane was incubated with the goat anti-mouse IgG (H+L) HRP conjugate diluted (1:3000 v/v) in PBS for 1 h at RT, and specific immunoreactive bands were visualized with a mixture of TMB substrate.

The inventors identified anti-HERV-K mAb 6H5 heavy chain CDRs (H-CDR1 30-35, H-CDR2 47-58, H-CDR3 93-101), and light chain CDRs (L-CDR1 30-36, L-CDR2 46-55, and L-CDR3 89-96) and grafted them onto selected human frameworks (FRs) showing the highest amino acid sequence identity to optimize the humanization of the given antibodies. Human immunoglobulin germline sequences showing the highest amino acid sequence similarity in FRs between human and mouse VH and VL were identified independently from the V-quest. (http://www.imgt.org/IMGT_vquest) and IgBLAST (http://www.ncbi.nlm.nih.gov/igblast) servers. The amino acid sequences in FRs of mouse VH and VL that differ from consensus human FRs were substituted with human residues, while preserving mouse residues at positions known as Vernier zone residues and chain packing residues. The clone of VH and VL chains of candidate humanized antibody genes were amplified and synthesized. The gene encoding the scFv, which includes a VH-linker-VL with a standard 20 amino acid linker (Gly4Ser)3 GGGAR, was inserted into a pET based vector (PAB-myc) containing a pelB promotor for controlling periplasmic protein expression (Novagen, Madison, WI) along with a 6×histidine tag at the C-termini for purification by metal affinity chromatography and a myc tag to facilitate rapid immunochemical recognition of the expressed scFv. The correct sequences of the scFv plasmid were used for soluble protein production in the periplasmic compartment. Two hu6H5 clones (FWJ1 and FWJ2) were selected and binding affinities to antigen were determined. Both clones were able to bind antigens produced from recombinant HERV-K Env surface fusion protein (KSU) and lysates from MDA-MB-231 breast cancer cells.

HuVH or HuVL with human IgG1 was cloned into a pcDNA 3.4 vector to produce VH-CH (human IgG1) or VL-CL (human Kappa). The plasmids were transiently transfected into Expi293 cells for mammalian expression. The ratio of H chain vs. L chain plasmids is 2:3. A Western blot was used to detect the VH chain and VL chain of the humanized anti-HERV-K antibody in an SDS-PAGE gel under reducing conditions. A 49 KDa molecular mass for the VH chain and a 23 KDa molecular mass for the VL chain was detected.

Size-exclusion chromatography (SEC) separation by size and/or molecular weight was further used to determine protein expression. Only two peaks were detected and the concentration of peak 2 was greater than 99% of the total combined size of peaks 1 and 2. Finally, the humanized 6H5 antibody (Purity>95%) with an endotoxin level<1 EU/mg was used to determine antitumor effects in vitro and in vivo.

An ELISA assay was used to compare antigen binding sensitivity and specificity of hu6H5 vs. m6H5. No significant difference between these two parameters was detected.

Apoptosis assays were used to determine the cytotoxicity of mouse and humanized anti-HERV-K antibodies toward cancer cells. Cancer cells including MDA-MB-231-pLVXK (231K) (a breast cancer cell line transduced with pLVX vector that expresses HERV-K env protein) or MDA-MB-231-pLVXC (231C) (the same breast cancer cell line transduced with pLVX empty vector) were treated with either m6H5 or hu6H5 (1 or 10 μg per ml) for four hours or sixteen hours. Annexin V and 7AAD were used to determine the percentage of apoptotic cells.

Live/dead cell viability assays were used to assess induction of cell death after anti-HERV-K antibody treatment. MDA-MB-231 cells were seeded overnight in 24-well plates. Cells were treated with various antibodies (10 μg/ml) and incubated for sixteen hours at 37C in a cell culture incubator. Calcein Am (4 μl/10 ml media) and Eth-D1 (20 μl/10 ml media), 200 μl per well, were then added and cells were incubated for thirty minutes at room temperature. EthD-1 penetrates cells with membrane damage and binds to nucleic acids to produce red fluorescence in dead cells. Live cells (green color; Calcein Am) and putative dead cells (red color; EthD-1) were identified using the co-stained Live/Dead Viability Assay. Human IgG or mouse IgG were used control. No red fluorescent cells were observed after treatment with control human or mouse IgG. However, red fluorescent cells were observed in cells treated with humanized or mouse 6H5 anti-HERV-K antibodies.

An MTT (3-(4,5-dimethylthiazol-2-yl)-2,5-diphenyltetrazolium bromide) assay was used to confirm that hu6H5 can inhibit cancer cell growth.

ADCC was used to determine the mechanism of antibody-induced cell killing. Greater ADCC lysis of cancer cells were observed in cells treated with hu6H5 than with m6H5, with increasing percentages of PBMCs.

Flow cytometry was used to determine if hu6H5 can downregulated the expression of p-ERK, Ras, and SIRT-1. 231 C (control) or 231 K (HERV-K transduced) cells were treated with 10 μg per ml of hu6H5 for 16 hr. The expression of HERV-K, SIRT-1, p-ERK and Ras in both cells was determined under perm and non-perm conditions. Down-regulated expression of HERV-K, p-ERK, Ras, and SIRT-1 was demonstrated in 231 K treated with hu6H5 or 231C.

Mice were inoculated with 231K or 231C cells (2 million by subcutaneous injection). The mice were then treated with hu6H5 antibody (n=3; 4 mg/kg, twice per week). Tumor growth was monitored and measured three times per week, and mouse survival was determined. Treatment of the mice with hu6H5 led to longer survival than treatment of another cohort of mice with the control antibody (n=4). Shorter survival was observed in mice inoculated with 231K cells than in mice inoculated with 231C cells, which indicates that overexpression of HERV-K in breast cancer cells shortens tumor-associated survival.

pLVXK is an HERV-K expression vector, and MDA-MB-231 pLVXK are MDA-MB-231 cells that were transduced with pLVXK. Likewise, pLVXC is control expression vector only, and MDA-MB-231 pLVXK are MDA-MB-231 cells that were transduced with pLVXC. NSG female mice (8-week-old), were inoculated with MDA-MB-231 pLVXC (231-C; subcutaneous, 2 million cells) vs, MDA_MB-231 pLVXK (231-K; subcutaneous, 2 million cells). On day 6, mice were treated with hu6H5 (4 mg/kg intraperitoneal, twice weekly for 3 weeks). Tumor growth was monitored and measured every other day. Higher survival was demonstrated in mice bearing 231-C and 231-K cells treated with antibodies. Tumor and lung tissues were collected from each mouse. Larger lymph nodes were detected in some mice bearing 231-K cells but not in mice bearing 231-C cells.

Hematoxylin and eosin (H&E) staining was further used to assess morphological features of tumor tissues and tissues from other organs (lungs and lymph nodes). Tumor viability and tumor necrosis were quantitated by a pathologist by measuring the tumor areas by H&E staining. HERV-K specific T cells (patient 369; IDC) were mixed with autologous mammosphere cells (40:1 ratio) in microengraving plate wells for four hours; live and dead tumor cells, and CD8+. α and β TOR sequences from HERV-K specific TILs. Humanized antibody treatment resulted in smaller tumor volumes, less tumor focality and number, less infiltrative borders, and decreased mitotic activities. A reduced percentage of tumor viability was observed in mice bearing 231-C cells or in 231-K cells treated with antibody. Reduced tumor variability was demonstrated in 231C or 231K cells treated with hu6H5, relative to their controls. Anti-Ki67 and anti-HERV-K mAb were used. Reduced tumor viability was demonstrated in mice treated with hu6H5 (20%; bottom panel) compared with control. The antibody treatment groups were more uniform in appearance, with less pleomorphic nuclei and smaller nucleoli, and tumor-infiltrating lymphocytes were significantly increased in number.

Metastases to lung and lymph nodes were observed in mice inoculated with 231K cells. Metastases to lung or lymph nodes were observed only in mice inoculated with 231K cells. Reduced tumor viability and increased tumor necrosis was detected in lung of mice inoculated with 231K cells and treated with hu6H5. Visibly enlarged lymph nodes were seen in mice inoculated with 231K, but not in mice inoculated with 231C cells. Reduced tumor viability and increased tumor necrosis were detected in lymph nodes from mice inoculated with 231K cells and treated with hu6H5 (KAB) (B18; 40%) vs 231K cells with no added antibody (KCON) (B26>95%). These results show that HERV-K expression is a causal factor for tumor development, and especially for metastasis to distant organ sites. Importantly, our humanized anti-HERV-K antibody can reduce tumor viability, increase tumor necrosis, and decrease metastasis to the lungs and lymph nodes.

### Example 5

### Efficacy of a Bispecific T Cell Engager (BiTE) Targeting HERV-K

A BITE directed against T cell CD3 or CD8 and the tumor-associated antigen HERV-K was produced, comprised of antibodies targeting either CD3 or CD8 and HERV-K. This BiTE was shown to elicit interferon-gamma (IFN gamma) cytotoxic activity towards MDA-MB-231 breast cancer cells expressing major histocompatibility class (MHC) molecules loaded with HERV-K epitopes, with 20-30-fold increases in IFN gamma expression after treatment with the BiTE.

A BITE is a recombinant protein built as a single-chain antibody construct that redirects T cells to tumor cells, and that does not require expansion of endogenous T cells through antigen-presenting cells. See Kontermann & Brinkmann, Drug Discovery Today (2015). BiTE molecules can be administered directly to patients and BITE-mediated T cell activation does not rely on the presence of Major Histocompatibility Complex class I molecules, as does CAR. Given the success of targeting HERV-K Env as a tumor-associated antigen (TAA), and that nearly all breast cancer cell lines express Kenv protein, the inventors hypothesize that a BiTE specific for Kenv and CD3 (K3Bi) effectively treats metastatic disease as did K-CAR. The inventors have designed and synthesized a K3Bi that has dual specificity for Kenv and CD3. Thus, T cells are directed to target HERV-K+ tumor cells. The inventors have generated, purified, and validated the K3Bi and a CD8 BiTE (K8Bi). This was done using the mAb 6H5 that was also used in the CAR construct (see Zhou et al., Oncoimmunology, 4, e1047582 (2015)), and OKT3, an antibody against human CD3 previously used in other BiTEs, which was humanized and connected with a flexible linker plus two C-terminal epitope tags (MYC and FLAG) for purification and staining. A CD8 single chain antibody (scFv) obtained from OKT8 hybridoma cells was generated in the inventors' lab and used to produce K8Bi (VL-VH6H5 linker VH-VLCD8-MYC and FLAG). K3Bi and K8Bi were cloned into the pLJM1-EGFP Lenti or pGEX-6P-1vector for recombinant protein expression. The capacity of the K3Bi or K8Bi to bind to T cells and HERV-K+ breast cancer cell lines was determined by several immune assays. The inventors found that increased numbers of target cells bound to BiTE with increased BiTE concentrations.

The inventors also examined the capacity of the K3Bi to induce T cell activation, proliferation, production of cytokines, and lysis of target tumor cells. Bulk PBMCs (50,000 per well) from healthy controls co-cultured with K3Bi (0, 1, 10, 100, and 1,000 ng/ml) and tumor cells (5,000 per well) to achieve effector cell:target cell ratios of 10:1 as described in Zhang et al., Cancer Immunol. Immunother., 63, 121-132 (2014). PBMC+ MCF-7⁺ K3Bi exhibited increased cancer cell killing compared PBMC+ MCF-7 without K3Bi. An LDH release assay was used for detection of cell viability and cytotoxicity, as the inventors did previously. See Zhou et al., Oncoimmunology, 4, e1047582 (2015). Enhanced IFNγ production, assayed by ELISA was observed in MDA-MB-231, MDA-MB-468, and MCF-7 cells treated with K3Bi. Untreated cells, PBMC only, or BiTE only were used as controls and no IFNγ production was observed in these control groups.

Treatment of immunodeficient NSG mice bearing HERV-K positive MDA-MB-231 breast cancer cells with PBMCs and CD3 HERV-K BiTE plus IL-2 or CD8 HERV-K BiTE plus PBMCs plus IL-2 resulted in greatly decreased tumor growth.

### Example 6

Staining Results of Normal Donor PBMC's Traduced with CAR-A and CAR-B Lentiviral Vectors

PBMCs from normal donors were transduced with two CAR-T lentiviral vector constructs, K-CAR-A (CAR-A) or K-CAR B (CAR-B). pWPT-GFP with psPAX2 and pMD2 g. VH-VLhu6H5-CD8-CD28-4-1BB-CD3zeta. The protocol to generate HERV-Kenv CAR-T cells by an alternate to the Sleeping Beauty transduction process, namely lentiviral transduction, is as follows:
1. Thaw PBMCs (2×107) and deplete monocytes by plastic adherence (one hour's incubation at 37° C., 5% CO2).
2. Culture monocytes depleted PBMCs in RPMI 1640 supplemented with 10% FBS, 100 U/mL penicillin, 100 μg/mL streptomycin (complete medium). Stimulate T cells with anti-CD3/CD28 beads in a 3:1 bead:cell ratio with 40 IU/mL IL-2 for twenty-four hours.
3. Transduce activated T cells with CAR-A or CAR-B lentiviral particles (CD19 CAR as control).
4. Twenty-four hours after transducing, culture T cells in complete medium containing 300 IU/mL IL-2 and γ-irradiated (100 Gy) MDA-MB-231-Kenv (Kenv is the envelope protein of HERV-K) aAPC at a 2:1 aAPC/T cell ratio to stimulate CAR-T cell proliferation. Use γ-irradiated K562-CD19 as the control aAPC.
5. Remove Anti-CD3/CD28 beads on day 5. Replenish CAR-T cells with fresh media containing IL-2 every two-three days.
6. Use CAR-T cells to perform further experiments when proliferation show a decrease from log-phase.

The CAR-A or CAR-B transduced cells were co-cultured with γ-irradiated (100 Gy) MDA MB 231 antigen presenting cells. Soluble IL-2 cytokine (50 U/ml) was added every other day. On day 14 the cells were harvested for staining. They were stained first for twenty minutes at 4° C. with a 1:1000 dilution of BV450 live and dead stain. After twenty minutes, the cells were washed and stained with K10-AF 488 protein (1 μg/ml), CD4 Amcyan, CD3 Pe cy7, and goat anti human IgG Fc AF 594 antibodies according to manufacturers' recommendations for thirty minutes at 4° C. and washed with PBS. The cells were fixed with 4% PFA for 15-30 mins and washed before analyzing in a flow cytometer. The samples were positive for GFP, as they were transfected with GFP+CAR-A/CAR-B.

The percentage of CD4+ cells was determined by gating those populations that were negative for BV450 and positive for respective colors. The percentage of CD4-(called CD8+ cells) were gated by selecting those populations that were negative for BV450 and negative for CD4 Amcyan color. The results show that the percentage of CD4+ PBMC's transduced with CAR-A/CAR-B that get stained with K10 labelled AF488 protein are higher than the percentage of naïve T cells that get stained with K10 labelled AF488 protein. This shows that T cells transduced with CAR-A or CAR-B are stained with the HERV-K10 protein.

T cells expressing a lentiviral CAR expression vector that bears a humanized or fully human HERV-K scFv will effectively lyse and kill tumor cells from several different cancers. Humanized K-CARs expressed from lentiviral vectors are pan-cancer CAR-Ts.

### Example 7

### A HERV-K Specific Humanized Chimeric Antigen Receptor (K-CAR) Therapy

The inventors have produced a humanized single chain variable fragment (scFv) antibody (EXAMPLE 1), which was able to bind antigens produced from recombinant HERV-K Env surface fusion protein (KSU) (EXAMPLE 3) and lysates from MDA-MB-231 breast cancer cells. A CAR produced from this humanized scFv is cloned into a lentiviral vector and is used in combination with therapies that include but are not limited to K-CAR T cells plus checkpoint inhibitors, proinflammatory cytokines such as interleukin (IL)-12 and IL-18, oncolytic viruses, and kinase inhibitors (including but not limited to p-RSK, p-ERK).

### Example 8

Identification of Human Therapeutic Antibodies (hTAbs) from Very Rare B Cells that Exhibit Strong Target Specificity and High Sensitivity

Generation of fully human therapeutic antibodies from the human adaptive immune system: To directly use B cells from breast cancer patients as a source of high-affinity antibodies, the inventors performed an indirect ELISA or immunoblot with HERV-K Env recombinant fusion protein, which the inventors used to detect anti-HERV-K Env specific responses from several different breast cancer patients. Patients with higher titers of anti-HERV-K antibodies were selected for single B cell experiments. PBMCs from breast cancer patients were polyclonally activated: (1) using irradiated 3T3-CD40L fibroblasts for a period of two weeks. This method can efficiently stimulate and expand CD40-B cells to large numbers in high purity (>90%) and induce secretion of their antibodies; and (2) ex vivo with recombinant human IL-21, IL-2, soluble CD40 ligand and anti-APO1 for four days. This second method can enable secretion from the highest percentage of B cells using minimal culture times. IL-21 is known to promote the differentiation to antibody-secreting cells. IL-2 stimulation in vitro can trigger human plasma cell differentiation, which requires appropriate T cell help to reach the induction threshold. sCD40L engages with CD40 expressed on the cell surface of B cells to mimic T cell-mediated activation. Because activation also induces cell death, anti-APO1 is used to rescue B cells from Fas-induced apoptosis Few cytotoxic B cells were detected.

Development of a platform to determine the binding kinetics and cell-to-cell interactions of every cell in a microwell slab. Details of the microengraving process, which enables the screening and monitoring of B cell interactions over time to enable single-cell cloning of antibody-producing B cells. The arrays of nanowells in polydimethyl siloxane (PDMS) are fabricated, and cells from mammospheres from patient breast tumor tissues produced and cultured in the inventors' lab were used as targets for determining the efficacy of breast cancer cell killing. B cells and mammosphere cells (1:1 ratio) from the same donor were loaded onto a nanowell array (one cell per well) and the cells allowed to settle via gravity. A dead tumor cell (red color) and B cell appear in the same well to indicate that the B cell was able to kill the tumor cell. The anti-HERV-K antibody produced by this B cell was detected in the same position of the glass cover slide. The single B cell was then picked by a CellCelector for RT-PCR. Our results show that HERV-K specific memory B cells exhibited anti-HERV-K antibody expression as well as cytotoxicity toward their autologous mammosphere cells.

Therapeutic antibody discovery using an in vivo enrichment (IVE) adaptation: The platform will enable isolation of antibodies that not only bind target cancer cells but can also kill the cells. It will also enable the use of normal donors without memory B cells instead of breast cancer patient donors to generate hTAbs. Since B cells able to produce therapeutic antibodies for treatment are extremely rare even after ex vivo enrichment, the inventors developed the following platform to identify very rare hTAbs:

ELISA was used to detect the anti-HERV-K antibody titers in the mice. Higher titers of antibodies were detected in mice treated with KSU Env protein regardless of CpG or CDN status. Anti-HERV-K antibody titers were detected by ELISA in HTM models inoculated with MDA-MB-231 (HTM1) or MDA-MB-468 (HTM2) and with HM (1-2) immunized with HERV-K SU Env protein using anti-human IgG mAb.

Groups (N=10/group) of wild type Balb/c mice (female, 6-week-old) are immunized on day 1 and boosted on week 3 and week 5. ELISPOT are used to determine IFNγ secretion by CD8⁺ T cells obtained from immunized mice. ELISA assays are used to detect the titers of anti-HERV-K IgG in immunized mouse sera.

### Example 8.1

Adapt an in vivo enrichment technique (IVE: ˜ 20-fold enhancement) in SCID/beige mice, allowing for rapid expansion and B cell activation, with a goal of producing large numbers of antigen-specific plasmablasts. This platform will produce fully human antibodies from B cells in as short a time as eight days. As a proof of principle, the inventors developed an IVE technique to produce fully human anti-Zika antibodies in hybridoma cells generated from splenocytes on day 8 fusion with MFP-2 partner cells.

Recently, humanized mice (HM) and human tumor mice (HTM) were successfully generated by intravenous injection of CD34+ cells (1-2×10⁵/mouse) for HM generation and immunization with HERV-K SU or PD-L1 recombined fusion proteins. The inventors also co-implanted CD34+ hematopoietic stem cells with 5×10⁴-3×10⁶ breast cancer cells triple negative breast cancer patient derived xenografts (TNBC PDX cells, or MDA-MB-231 or MDA-MB-468 TNBC cells) in the mammary fat pad for HTM generation. The percentage of hCD19 or hCD45 cells is higher in mice after a longer period of post-inoculation with CD34 cells. Exposure to antigen was associated with HERV-K expression in the tumor, and a higher antibody titer was detected (HTM 2:40 days vs. HTM 1:30 days). Importantly, this indicates that HTMs can produce anti-HERV-K antibodies in mice inoculated with breast cancer cells. This finding prompted us to explore the use of HM or/and HTM to generate fully hTAbs, and especially to use normal donors who were never exposed to antigen. NSG mice, which lack T-cell, B-cell and NK cell activity, are considered as ideal candidates to establish HM. Mice with a higher engraftment rate of human CD45+ cells than was seen in earlier studies, without any significant toxicity, were developed recently.

Protocol 1. For donors who have cancer with a higher titer of antibodies, the inventors use the protocol using HM instead SCID/beige mice. PBMCs (50×10⁶) from breast cancer patients are polyclonally activated by IL-21, IL-2, soluble CD40 ligand and anti-APO1, and premixed with antigens (HERV-K or PD-L1; 100 μg). B cells isolated from the above PBMCs using an EasySep™ Human B Cell Enrichment Kit (Stemcell Technologies) by negative selection are co-injected with CD34 cells in the mice treated with busulfan. See scientific reference 61. (Fisher: 30 mg/kg intraperitoneally) on day 0. Mice are treated with cytokine cocktails (days 1, 4, and 7) and boosted by antigens on day 2. This protocol can be completed relatively quickly (8 days).

Protocol 2. For normal donors who do not have cancer and who have no memory B cells, the inventors use Protocol 1 with modifications: Mice are treated with cytokine cocktails (days 1, 7, and 14) and boosted by antigens on day 14 and day 21. Sera are collected from mice and binding affinity is tested by ELISA. After increased antibody titers are detected, spleens are harvested, analyzed, and used to make hybridomas. Higher antibody titers were detected in mice using IVE Protocol 2 on week 2.

### Example 8.2

After IVE, half of the spleen is harvested and used for flow cytometric analysis, microengraving and other analyses. Flow cytometric analysis of B cell surface and intracellular markers and CFSE labeling (Invitrogen CellTrace CFSE kit) is performed using the following: Anti-CD19 PECy5, anti-CD27 allophycocyanin, anti-CD38 PECy7, anti-IgG FITC, or anti-IgM PE isotype controls of mouse IgG1k conjugated to FITC, PE, PECy5, PECy7, Alexa 700, or allophycocyanin (all from BD Bioscience). Negative magnetic immunoaffinity bead separation (Miltenyi Biotec) is used to isolate total CD19+ B cells from spleen and stimulate with CpG2006 (10 ng/ml; Oligos, Inc.) in the presence of recombinant human B cell activating factor (BAFF; 75 ng/ml; GenScript), IL-2 (20 IU/ml), IL-10 (50 ng/ml), and IL-15 (10 ng/ml) (all from BD Biosciences) for seventy-two hours. Tumor-killing B cells directly from Protocol 1 or 2 are determined using our multi-well microengraving platform (up to 400,000 wells), with their autologous tumor cells or HERV-K+TNBC cells as target cells. Cells that not only produce antibodies but are also able to bind antigen and kill cancer cells are determined.

### Example 8.3

The inventors then develop human hybridoma cells to ensure long-term antibody availability. To develop a fully human hybridoma, MFP-2 cells are used as a partner to generate hybridomas with the remaining half of the spleen using ClonaCell™-HY (Stemcell Technologies Inc.) following their protocol. Polyethylene glycol (PEG) is used for fusing human lymphocytes with MFP-2 cells and a methylcellulose-based semi-solid media in this kit is used for cloning and selection of hybridoma cells. The clones that grow out after selection are pipetted into 96-well plates and screened for reactivity to HERV-K Env protein by ELISA. The positive clones' isotypes are determined using a Human IgG Antibody Isotyping Kit from Thermo Fisher Scientific. The clones are then adapted to serum-free media conditions and expanded. Hybridoma supernatant is harvested, and antibody is purified using Hi-Trap protein A or protein G columns, depending on the isotype of the human antibody. Protein A columns are known to have high affinity to antibodies of the isotype-IgG1, IgG2, and IgG4, and variable binding to antibodies of the isotype IgM, whereas Protein G columns are known to exhibit high binding to antibodies of the isotype-IgG1, IgG2, IgG3, and IgG4, but do not bind IgM antibodies.

### Example 8.4

The inventors evaluate the antitumor efficacy of candidate B cells obtained from the above protocols in vitro, including effects on cell growth, proliferation, and apoptosis, as the inventors do routinely in our lab. In vivo studies to evaluate the efficacy of the hTAbs in immunodeficient mouse models are also done to evaluate efficacy, using breast cancer cell lines and primary tumor cells, and compared with matched uninvolved control breast cells.

### Example 9

### Combination Therapy

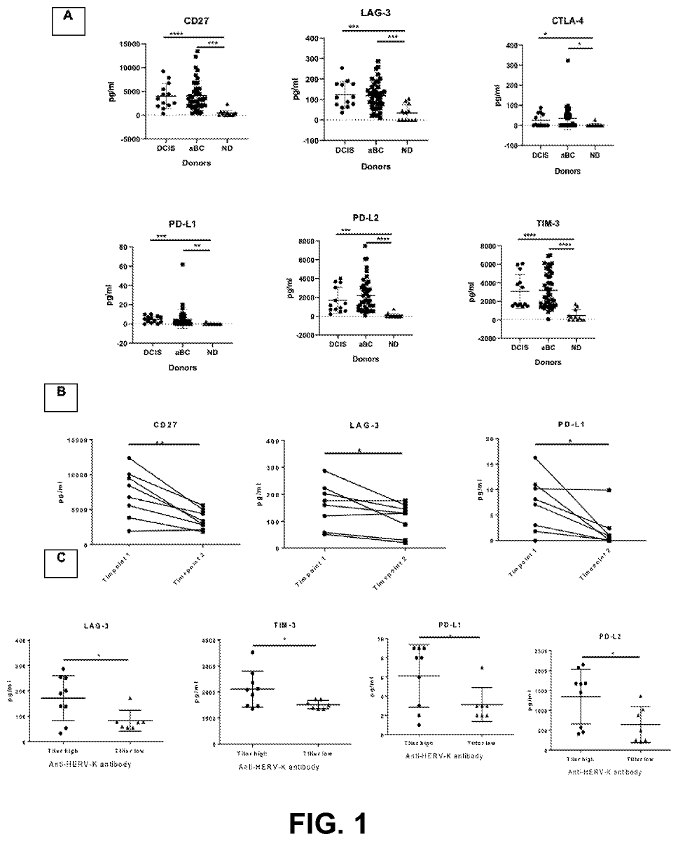

The inventors' breast cancer data from strongly support the potential for combination therapy approaches involving HERV-K. Humanized and fully human antibodies targeting HERV-K will therefore enhance checkpoint blockade antibody treatment efficacy. Effective combined cancer therapies include but are not limited to combinations of (a) HERV-K hTAb (1.5 mg/kg), (b) K-CAR, (c) K-BiTE, (d) HERV-K shRNAs or CRISPR/Cas9 genome editing technology to knock down HERV-K gene expression, (e) or preventative or therapeutic HERV-K vaccines, including full-length and truncated HERV-K Env proteins and HERV-K Env peptides, and (a) anti-ICP antibody (FIG. 1), (b) cancer chemotherapy, (c) 5-azacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion, (f) induction of S or G2 phase cell cycle arrest, (g) inhibitors of PI3K/AKT/mTOR or MAPK/ERK signaling pathways, or (h) signal transduction to HIF1a.

### Example 10

Sequence Data Anti-CD3 and CD8 biTE—Alignment

```
TABLE 4
H3 TNTCAGGTGCAGCTGAAGCAGTCTGGGGCAGAGCTTGTGAAGCCAGGGGCCTCAGTCAAG 60
H6 TTTGAGGTCCAGCTGCAGCAGTCTGGGGCAGAGCTTGTGAAGCCAGGGGCCTCAGTCAAG 60
H7 --TCAGGTCCAACTGCAGCAGCCTGGGGCAGAGCTTGTGAAGCCAGGGGCCTCAGTCAAG 58
H1 TTTGAGGTCCAGCTGCAGCAGTCTGGGGCAGAGCTTGTGAAGCCAGGGGCCTCAGTCAAG 60
      * **** **.***.***** **************************************
H3 TTGTCCTGCACAGCTTCTGGCTTCAACATTAAAGACACCTATATACACTTCGTGAGGCAG 120
H6 TTGTCCTGCACAGCTTCTGGCTTCAACATTAAAGACACCTATATACACTTCGTGAGGCAG 120
H7 TTGTCCTGCACAGCTTCTGGCTTCAACATTAAAGACACCTATATACACTTCGTGAGGCAG 118
H1 TTGTCCTGCACAGCTTCTGGCTTCAACATTAAAGACACCTATATACACTTCGTGAGGCAG 120
      ************************************************************
H3 AGGCCTGAACAGGGCCTGGAGTGGATTGGAAGGATTGATCCTGCGAATGATAATACTTTA 180
H6 AGGCCTGAACAGGGCCTGGAGTGGATTGGAAGGATTGATCCTGCGAATGATAATACTTTA 180
H7 AGGCCTGAACAGGGCCTGGAGTGGATTGGAAGGATTGATCCTGCGAATGATAATACTTTA 178
H1 AGGCCTGAACAGGGCCTGGAGTGGATTGGAAGGATTGATCCTGCGAATGATAATACTTTA 180
      ************************************************************
H3 TATGCCTCAAAGTTCCAGGGCAAGGCCACTATAACAGCAGACACATCATCCAACACAGCC 240
H6 TATGCCTCAAAGTTCCAGGGCAAGGCCACTATAACAGCAGACACATCATCCAACACAGCC 240
H7 TATGCCTCAAAGTTCCAGGGCAAGGCCACTATAACAGCAGACACATCATCCAACACAGCC 238
H1 TATGCCTCAAAGTTCCAGGGCAAGGCCACTATAACAGCAGACACATCATCCAACACAGCC 240
      **********************************************************
H3 TACATGCACCTCTGCAGCCTGACATCTGGGGACACTGCCGTCTATTACTGTGGTAGAGGT 300
H6 TACATGCACCTCTGCAGCCTGACATCTGGGGACACTGCCGTCTATTACTGTGGTAGAGGT 300
H7 TACATGCACCTCTGCAGCCTGACATCTGGGGACACTGCCGTCTATTACTGTGGTAGAGGT 298
H1 TACATGCACCTCTGCAGCCTGACATCTGGGGACACTGCCGTCTATTACTGTGGTAGAGGT 300
      ***********************************************************
H3 TATGGTTACTACGTATTTGAC-CACTGGGGCCAAGGCACCAC---TCTCACANTNNCNNN 356
H6 TATGGTTACTACGTATTTGAC-CACTGGGGCCAAGGCACCAC---TCTCACANTNNCNNN 356
H7 TATGGTTACTACGTATTTGAC-CACTGGGGCCAAGGCACCAC---TCTCACATNTNNNTN 354
H1 TATGGTTACTACGTATTTGACTCACTTGGGCCAAGGCNNTNNNNNTNNCANNNTNNNNNN 360
      ********************* **** ********** .**...* *.*
H3 N- 357 (SEQ ID NO: 24)
H6 A- 357 (SEQ ID NO: 26)
H7 -- 354 (SEQ ID NO: 28)
H1 NN 362 (SEQ ID NO: 22)
```

```
TABLE 5
K4 TTTTGACATTGTGCTGACCCAATCTCCTGCTTCCTTAGCTGTATCTCTGGGGCAGAGGGC 60
K6 TNTTGATATTGTGCTAACTCAGTCTCCTGCTTCCTTAGCTGTATCTCTGGGGCAGAGGGC 60
K8 ---TGACATCCAGCTGACTCAGTCTCCTGCTTCCTTAGCTGTATCTCTGGGGCAGAGGGC 57
            *** **:***.** **.***************************************
K4 CACCATCTCATACAGGGCCAGCAAAAGTGTCAGTACATCTGGCTATAGTTATATGCACTG 120
K6 CACCATCTCATACAGGGCCAGCAAAAGTGTCAGTACATCTGGCTATAGTTATATGCACTG 120
K8 CACCATCTCATACAGGGCCAGCAAAAGTGTCAGTACATCTGGCTATAGTTATATGCACTG 117
      **********************************************************
K4 GAACCAACAGAAACCAGGACAGCCACCCAGACTCCTCATCTATCTTGTATCCAACCTAGA 180
K6 GAACCAACAGAAACCAGGACAGCCACCCAGACTCCTCATCTATCTTGTATCCAACCTAGA 180
K8 GAACCAACAGAAACCAGGACAGCCACCCAGACTCCTCATCTATCTTGTATCCAACCTAGA 177
      **********************************************************
K4 ATCTGGGGTCCCTGCCAGGTTCAGTGGCAGTGGGTCTGGGACAGACTTCACCCTCAACAT 240
K6 ATCTGGGGTCCCTGCCAGGTTCAGTGGCAGTGGGTCTGGGACAGACTTCACCCTCAACAT 240
K8 ATCTGGGGTCCCTGCCAGGTTCAGTGGCAGTGGGTCTGGGACAGACTTCACCCTCAACAT 237
      ***********************************************************
K4 CCATCCTGTGGAGGAGGAGGATGCTGCA-ACCTATTA-CTGTCAGCACATTA-GGGAGCT 297
K6 CCATCCTGTGGAGGAGGAGGATGCTGCA-ACCTATTA-CTGTCAGCACATTA-GGGAGCT 297
K8 CCATCCTGTGGAGGAGGAGGATGCTGCTGACCTATTAGCTGTCAGCACATTATGGGAGCT 297
      ***************************: ******* ************** ******
K4 TACACGTTCGGAGGGGGACCAAGCNNNNNNN--- 328 (SEQ ID NO: 30)
K6 TACACGNNGANAANNNNNNNNNNCNNNNTNNNNN 331 (SEQ ID NO: 32)
K8 TACACGTTCGGNNNNNTNNCTNNTNCNNNNNNNC 331 (SEQ ID NO: 34)
      ******
```

Order of the IgG domains: VL-VH6H5 - - - VH-VLhuCD3 or CD8⁺ c-myc tag+FLAG or vL-VHhu6H5 - - - VH-VLhuCD3 or huCD8⁺ c-myc tag+FLAG

CD8 BiTE: See SEQ ID NOs: 29-30.

CD3 BiTE: See SEQ ID NOs: 31-32.

Mice were immunized with 5 MAPs and sera were collected and tested by ELISA using various HERV fusion proteins. Only HERV-K SU protein was positive. Hybridoma cells were generated from the mice immunized with 5 MAPs and a scFv was selected having the sequence below. For scFv against MAPs of HERV-K (sequence for anti-HERV-K mAb), see SEQ ID NOs: 45-46.

### Example 11

Humanized Antibodies Targeting HERV-K that can be Used for ADCs to Deliver the Drugs into Cancer Cells and Tumors

Recombinant gelonin (r-Gel) toxin was conjugated with 6H5. r-Gel was detected in OVCAR3, SKBr3, MCF-7, and MDA-MB-231 cells after one hour internalization using anti-r-Gel antibody. Gold nanoparticles (GNPs) were detected after two hours incubation with naked GNP or 6H5-GNP by transmission electron microscopy (TEM) in MDA-MB-231 cells. GNPs were detected in MDA-MB-231 or SKBr3 of tumors isolated from mice twenty-four hours post-intravenous-injection with the 6H5-GNP or 6H5scFV-GNP using a silver enhancement assay. GNPs generate heat that kills targeted tumor cells when they are placed in a radiofrequency field.

### Example 12

### In Vivo Imaging of Anti-HERV-K Antibodies in Tumor Nodules of Mice

A higher density of 6H5 was detected in tumor nodules from mice twenty-four hours post-intravenous-injection with the anti-HERV-K-Alexa647 conjugate 6H5-Alexa647 (red color) by in vivo imaging using a Nuance system. This result supports the ability of the 6H5 antibody to specifically target tumors that express HERV-K.

### Example 13

T Cell Receptor (TCR) α/β Chains Generated from Cytotoxic HERV-K Specific Tumor Infiltrating Lymphocytes (TILs)

The inventors developed a high-throughput nanowell microengraving platform for detecting cytotoxicity of K-T cells at the single cell level. The nanowells are fabricated in polydimethyl siloxane (PDMS), allowing inexpensive, rapid, and repeatable fabrication from molds produced on silicon in photoresist using standard photolithography. T cells able to kill autologous tumor cells were identified and picked using the CellCelector™ system. Single isolated T cell colony isolates were co-cultured with HERV-K expressing DCs.

Each of the clonally expanded T cell colonies was picked and deposited into each well of a 96-well PCR plate. The inventors performed a multiplex PCR immediately for molecular analysis of paired αβ TCR chains using primer sets that are specific to the entire repertoire of functional TCR β-gene V-elements and TCR α chains. See Seitz et al., Proc. Natl. Acad. Sci. USA, 103, 12057-12062 (2006). Each band of PCR products was sequenced, and matching TCR sequences were checked using the IMGT database. The inventors confirmed the validity of this approach as both PBMCs (data not shown), and TILs yielded productive TCR αβ rearranged receptors (VP7; T IL+K-GST and #1-α4 and #5-α5) that were sequenced.

This platform enabling functional matching TCR sequences can be acquired from a small number of homogenous HERV-K specific T cell (K-T cell) populations from a single clonally expanded K-T cell.

### Example 14

### Generation of Primary T Cells or Mammosphere Cells

PBMCs were isolated from human breast cancer patients and controls. PBMCs were isolated by density gradient centrifugation with histopaque-107. TIL or normal tissue-infiltrating lymphocyte (NIL) cells were generated from tumor or uninvolved normal breast tissues, respectively. Analysis of subtypes of T cells from PBMCs, normal NIL, and TIL included determining percentages of CD8⁺ and CD4⁺ in CD3⁺ T cells by FACS.

TILs and NILs were cultured for 2-4 weeks in TIL culture medium containing high-dose IL-2. Primary breast cells were isolated from tumor or uninvolved breast tissues from breast cancer patients after collagenase and hyaluronidase digestion. Mammosphere cells were isolated from these tumor and uninvolved cells and were cultured in mammosphere medium for 2 weeks. TILs (Tumor) or NILs (Normal) cells were generated from tumor or uninvolved breast tissues from patients 361 (IDC+DCIS), 364 (IDC), or 370 (DCIS), after 14-day culture. Images were taken on day 14. Mammospheres were generated from patients 361 tumor (T), 369 tumors (T; invasive mammary carcinoma: IMC), or normal (N), and 370 tumor or normal breast tissues.

### Example 15

### Production of Functional HERV-K Specific T Cells

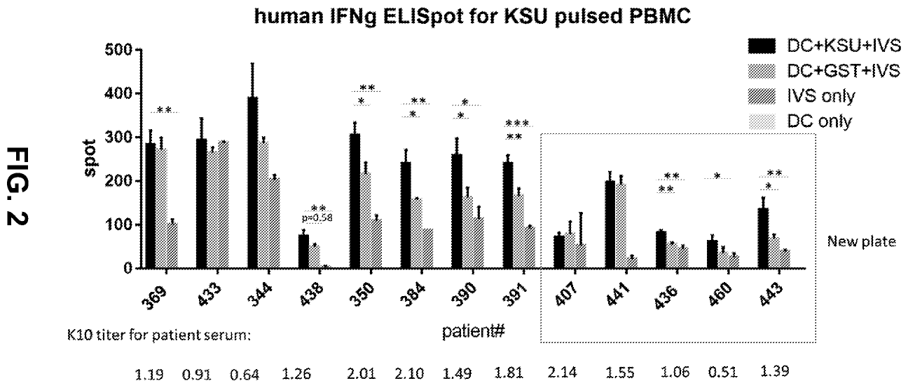

These data showed that HERV-K specific T cells (K-T cells) from tumor infiltrating lymphocytes or PBMCs exhibited secretion of IFNγ by ELISPOT (FIG. 2). K-T TIL cell cytotoxicity toward their autologous mammosphere cells was further demonstrated.

### Example 17

### Circulating Tumor Cells (CTCs) are HERV-K Positive Cells

Circulating tumor cells are considered the seeds of residual disease and distant metastases and their characterization and targeting would guide treatment options. To explore this possibility, multiple biomarkers (cytokeratin (CK) or HERV-K) were used to detect CTCs. These results show that HERV-K staining overlaps in many cases with staining of the serum tumor marker CK. HERV-K can be a CTC marker as well as a target for HERV-K antibody therapy.

These data provide evidence that HERV-K is a stem cell marker, and that targeting of HERV-K may block tumor progression by slowing or preventing growth of cancer stem cells. Targeting of HERV-K with circulating therapeutic antibodies or other therapies may also kill CTCs and prevent metastasis of these circulating cells to distant sites.

### Example 18

### Increased HERV-K Target Expression on Cancer Cells, Thereby Increasing the Efficiency/Efficacy of Cytotoxic or Immunotherapeutic Targeting

The expression of HERV-K was detected in several CRC cell lines and tissues, but not in benign colon tissues. Increased expression of HERV-K was observed in HCT15, SW48 and HCT116 cells treated with Poly I:C (TLR3 & RIG-I activator) or 5-Azacytidine (5-Aza, a demethylation agent). The forced overexpression of HERV-K with these agents that induce expression of HERV-K by innate immune response (Poly I:C treatment) or LTR hypomethylation (5-Aza) would provoke the cancer cells to increase production of a target that would make them more susceptible to targeted therapy to include targeted immunotherapy.

### Example 19

### HERV-K Cancer Vaccine Adjuvants

A major challenge in the cancer vaccine field is the efficient delivery of antigen/adjuvant to secondary lymphoid organs, where immune responses are orchestrated. Most of the protein-based vaccines using currently available adjuvants fail to promote a robust CD8+ T cell response, limiting their potential effectiveness. A priority of this study will be to identify adjuvants that elicit robust CD8+ T cell cytolytic responses against the tumor. For example, (1) CpG, a TLR9 ligand, is a promising adjuvant developed for vaccines because it elicits a strong Th1-biased immune response; 59 use of CpG was recently approved in humans; (2) cyclic dinucleotides (CDN) recognized by STING (STimulator of INterferon Genes) provoke an interferon response that is associated with tumor immunity. In clinical trials CDN is used as a cancer immunostimulator. Because of CDN's small size, which would allow the adjuvant to diffuse away from the antigen, which limits effectiveness of the vaccine, both will be incorporated into the squalene emulsion Addavax, which provides a depot-like effect to prolong release of antigen/adjuvant. The CpG are also relatively small and will be incorporated into Addavax+HERV-K Env to maintain antigen/adjuvant proximity. Addavax is like MF59, an adjuvant approved for use in Europe.

Recombinant HERV-K surface (SU) Env protein (0, 50, or 100 μg) mixed with CpG (10-20 μg; Adjuvant #1) or CDN (10-15 μg; Adjuvant #2), in Addavax (100 μl) are used to immunize mice. See TABLE 6.

TABLE 6
Amounts of antigen and adjuvant to be tested.

| Adjuvant | Dosing Schedule | Amt of Env (ug) | No mice |
|---|---|---|---|
| Adjuvant #1/2 | Single injection | 0 | 10 |
| CpG/CDN + | Mice harvested 2 weeks | 50 | 10 |
| Addavax | later | 100 | 10 |
| Adjuvant #1/2 | 3 rounds of | 0 | 10 |
| CpG/CDN + | immunizations, spaced 2 | 50 | 10 |
| Addavax | weeks apart | 100 | 10 |

The two adjuvants that produce the most IFNγ+spots in this screening assay are further optimized as described below. Groups (N=10/group) of wild type Balb/c mice (female, 6-week-old) are immunized on day 1 and boosted on week 3 and week 5. ELISPOT are used to determine IFNγ secretion by CD8+ T cells obtained from immunized mice. ELISA assays are used to detect the titers of anti-HERV-K IgG in immunized mouse sera.

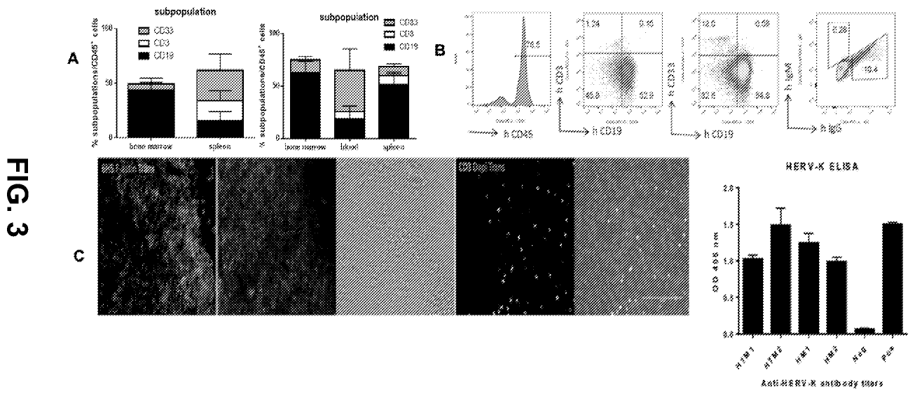

Optimal concentrations of antigen and adjuvant in the vaccine are determined by measuring both humoral and cellular immune response in immunized mice treated with increasing doses of HERV-K SU Env protein, with added CDN or CpG. Maximum tolerated dose (20-100 mg/kg, followed for 3 weeks) are investigated for effects on body weight or other clinical toxicity symptoms. These mice include humanized mice (HM) and human tumor mice (HTM) that have been successfully generated by intravenous injection of CD34+ cells (1-2×10⁵/mouse) for HM generation and immunization with HERV-K SU or treatment with PD-1 recombined fusion proteins. The inventors also co-implanted CD34+ hematopoietic stem cells with 5λ10⁴-3×10⁶ breast cancer cells triple negative breast cancer patient derived xenografts (TNBC PDX cells, or MDA-MB-231 or MDA-MB-468 TNBC cells) in the mammary fat pad for HTM generation. The percentage of hCD19 or hCD45 cells is higher in mice after a longer period of post-inoculation with CD34 cells. See FIG. 3.

The vaccine is delivered either subcutaneously (s.c.) for CpG or CDN or intraperitoneally (i.p.) for HMW poly (I:C), a TLR3/RIG-1 agonist that is currently undergoing clinical trials. This protocol activated both TLR3 and cytosolic RIG-1 sensors to maintain Type I IFN activity, allowing for sustained adjuvant activity. HERV expression activates the innate sensor response including RIG1, MDA5, and TLR3 in cytosol to activate the Type I IFN response. See Chiappinelli et al., Cell, 16, 974-986 (2015).

### Example 20

### Evaluating Baseline Immune Status in Relation to HERV-K Status in Breast Cancer Patients: Combined HERV-K and Immune Checkpoint Assays

Expression of soluble immune checkpoint proteins (ICPs) was determined by Luminex assay in breast cancer patients including DCIS and aggressive breast cancer vs. normal donors. A striking and previously unreported finding was a significantly enhanced expression of six circulating ICPs in the plasma of breast cancer patients (FIG. 1). A further finding was a marked drop in ICP levels in patients at six (Timepoint 2) or eighteen months post-surgery vs. pre-surgery (Timepoint 1). Importantly, a positive association between soluble ICP molecule levels and HERV-K antibody titers induced by HERV-K expression in the tumor was observed. Thus, HERV-K antibody titers can influence ICP levels in breast cancer. The expression of HERV-K can thus control immune responses of breast cancer patients.

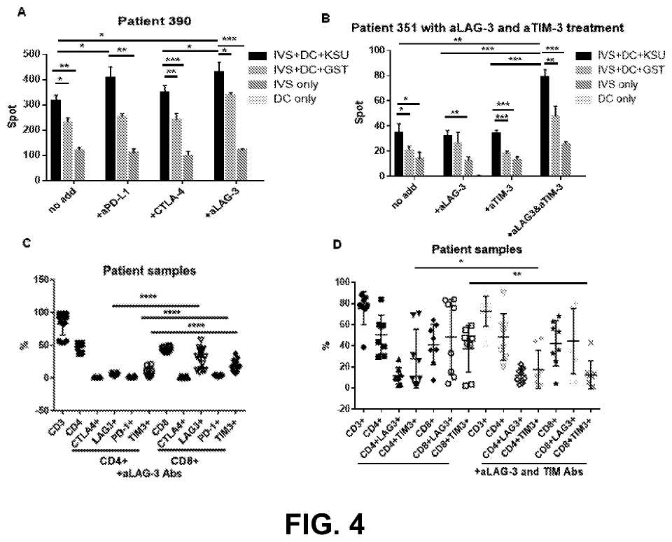

Enhanced spots of IFN-gamma with combinations of inhibitors of ICPs and HERV-K specific T cells: In an initial experiment in cancer patient #390 and #351, dendritic cells (DCs) that were transfected with HERV-K surface (SU) envelope (Env) protein had a much greater number of IFNγ spots than DCs transfected with a control GST protein (FIG. 4A), indicating a much greater immune response. When the PBMCs or TILs were also treated with anti-PD-L1, anti-CTLA-4, anti-LAG-3, and anti-TIM-3 antibodies the immune response was even stronger, especially using both anti-LAG-3 and anti-TIM-3 antibodies (FIG. 4B) in comparison to both anti-PD-1 and anti-CTLA-4 antibodies (data not shown). Thus, LAG-3 and TIM-3 exhaustion in the HERV K-T cells can be countered by anti-LAG3 and anti-TIM-3 therapy.

Several classes of K-specific T cells were determined by flow cytometry (FIG. 4C and FIG. 4D). The percentage of CTLA-4+ (1.25% and 1.05%) or PD-1+ (1.07% and 3.75%) is much lower than LAG-3 (4.16% and 31.87%) or TIM-3 (7.76% and 18.46%) in CD4+ (47.86%) and CD8+ T cells (43.41%; N=8), respectively. The TIM-3+CD8+ fraction showed a more significant decrease than the TIM-3+CD4⁺ fraction of K-specific T cells treated with anti-LAG-3 plus anti-TIM-3 antibodies (FIG. 4D).

These striking findings support the concept that HERV-K triggers an immune response that can be complemented by immune checkpoint protein therapy. Therefore HERV-K effectively convert breast cancer from cold into hot tumors if it combines with the correct checkpoint blockade therapy partners.

### Example 21

### HERV-K Breast Cancer Vaccines

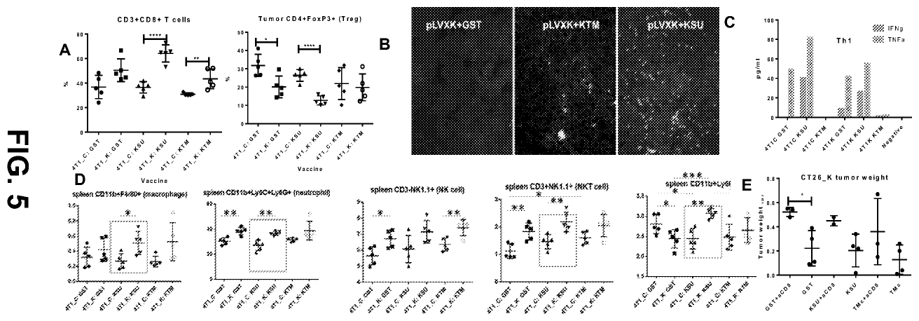

In vivo HERV-K models: Murine mammary tumor cells (4T1), melanoma cells (B16F10) or colon cancer cells (CT26) were engineered to express HERV-K Env34 to produce unique syngeneic models of HERV-K crucial for studying the role of the anti-tumor immune response (FIG. 5). This was accomplished by stably transfecting cells with pLVX-Kenv (full length HERV-K env, expressing both extracellular SU and TM domains) or pLVX vector only (control).

For cancer prevention vaccine studies, recombinant HERV-K SU or TM protein (0, 50, or 100 μg) is mixed with CpG (10-20 μg) or cyclic dinucleotides (CDN: 10-15 μg) in Addavax (100 μl). CDN, recognized by STING (STimulator of INterferon Genes), is used in clinical trials as a cancer immunostimulator. CDN provokes an interferon response that is associated with tumor immunity. This vaccine is used to immunize mice on weeks −5, −3, and −1, and 4T1pLVX-Kenv or 4T1pLVX (3×10⁵) cells are injected s.c. in the 4th mammary fat pad on day 0. Tumor growth is monitored, and tumor tissues are harvested. Significantly increased percentages of CD8 T cells infiltrated tumors of mice inoculated with 4T1-pLVXKenv (4T1_K) cells and immunized with either HERV-K full-length surface protein (KSU) or full-length TM protein (KTM), in comparison to mice inoculated with cells transduced with vector only (4T1_C) and immunized with KSU or KTM. GST protein was a control antigen. However, significantly decreased Treg cell percentages were detected in tumors from mice inoculated with 4T1_K than with 4T1_C cells and immunized with KSU (FIG. 5A), a change not observed for KTM immunosuppressive protein62 immunization. Interestingly, more CD8 T cells were detected in 4T1 (FIG. 5A) or B16F10-pLVXKenv (B16F10_K; FIG. 5B) tumors from mice immunized with KSU protein>KTM>GST. Also, IFNγ and TNF-α cytokines (FIG. 5C) were detected in mice immunized with KSU protein. Increased macrophage (FIG. 5D), neutrophil, NK, NKT cells, and myeloid-derived suppressor cells (MDSC) were detected in mice immunized with KSU than with KTM or GST, after challenge with tumor cells expressing HERV-K. In addition, increased tumor weights were observed in vaccinated mice inoculated with CT26_K cells and treated with anti-CD8 antibody (FIG. 5E). Cytokine array results revealed increased secretion of cytokines specific for KSU include IFN-gamma, TNF-alpha, IL-17A, IL5, and IL-4, whereas decreased secretion of cytokines specific for KSU are IL-23 and IL-12 was observed. Increased secretion of cytokines specific for KTM include IL-1, IL-6, and IL-2. The data indicate that CD8 T cells play a role in killing tumor cells expressing HERV-K in vaccinated mice.

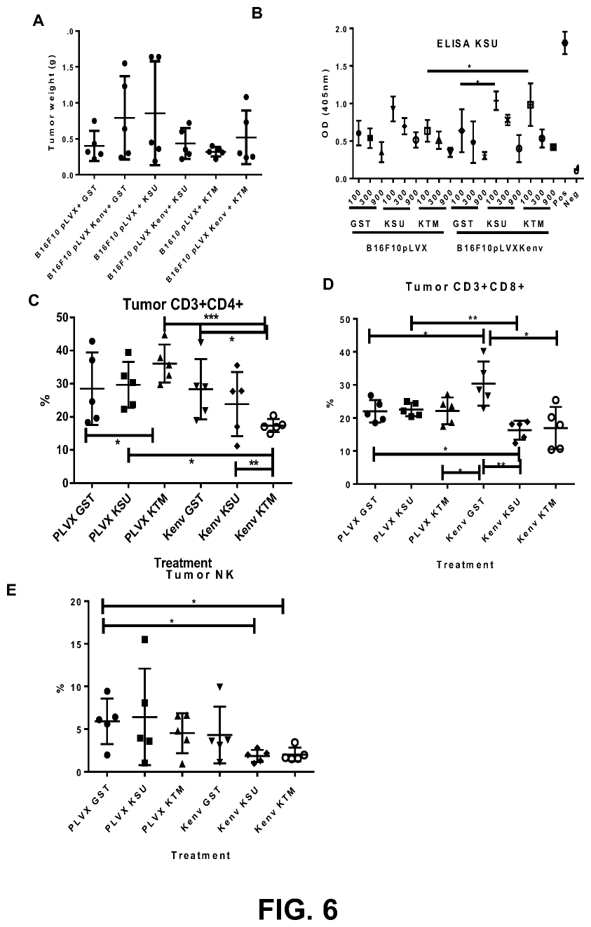

Using this model (FIG. 6), increased weight of pLVXKenv-transduced tumors relative to pLVX control cell tumors was observed in mice immunized with GST (2-fold increased weight). In contrast, the inventors observed reduced weight of pLVXKenv relative to pLVX tumors in mice immunized with KSU (50% reduced weight), showing the protective effect of KSU vaccination. This protective effect disappeared in mice immunized with the TM (1.65-fold increased tumor weight), indicating that the immunosuppressive domain (ISD) of TM62 may prevent an immune response to the vaccine.

For cancer vaccine treatment, CpG, a TLR9 ligand, is a promising adjuvant developed for vaccines because it elicits a strong Th1-biased immune response; 59 use of CpG was recently approved in humans. Because optimal vaccines tend to encapsulate antigen and adjuvant together, they are both taken up into the same APC, so the inventors have formulated these with an amphiphile (Amph), a liposomal mix that allows for conjugation of adjuvant and antigen together. Amph vaccines have provided a simple, broadly applicable strategy that simultaneously increases the potency and safety of subunit vaccines.63 BALB/c female mice (6 weeks old) are inoculated subcutaneously with 4T1_K (1×10⁵ cells) on day 0. Mice are treated with HERV-K surface protein (HERV-K SU) (100 μg), or with Amph-CpG (1.2 nmol) or CpG (1.2 nmol) on day 6, day 13, and day 19 after tumor inoculation (N=6/group). Tumor size is monitored longitudinally throughout the study. Spleen and tumor are collected for analysis of various cell populations as in FIG. 6.

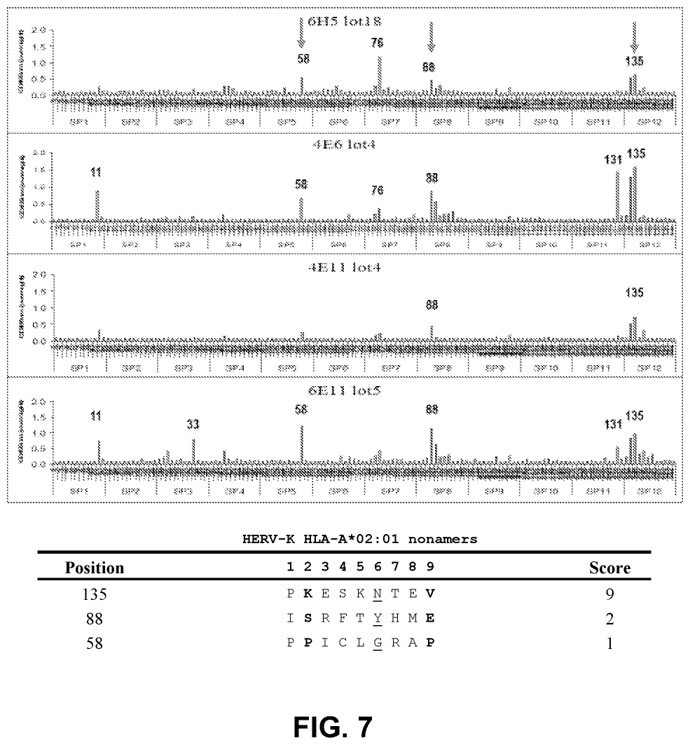

Peptide models: HERV-K peptide mapping: The inventors used peptide mapping to identify B cell specific peptides of the full-length HERV-K env protein. The inventors chemically synthesized 144 15-mer peptides covering the full-length HERV-K env gene sequence with an overlap of eleven amino acids. The 144 peptides were divided into twelve small pools (each consisting of twelve peptides). Anti-HERV-K Env mAbs were screened by ELISA for reactivity with individual peptides (FIG. 7). The inventors found that 3 peptides bound consistently to HERV-K mAbs from several lots (red arrows). These peptides are translated into HERV-K-specific vaccines, starting with peptide #135, which binds to all the anti-HERV-K mAbs.

Vaccine preparation: An N-terminal cysteine residue is added to peptide #135 to enable attachment of the peptide on the surface of keyhole limpet hemocyanin (KLH) protein. The peptide is coupled to the KLH carrier with the bifunctional cross-linker N-[γ-maleimidobutyryloxy]succinimide ester (GMBS), as described64. The vaccine is prepared by mixing HERV-K peptide conjugate in a 1:1 (vol/vol) ratio with Adju-Phos adjuvant (InvivoGen) in a final dose of 300 μl that contains 100 μg of conjugated peptide.

Vaccination protocol: NSG mice inoculated with human breast cancer cells in the fourth mammary fat pad are immunized with five subcutaneous injections of the vaccine, starting at 6-8 weeks of age, followed by the second injection 2 weeks later and thereafter on a biweekly schedule. Control NGS mice not inoculated with human breast cancer cells will receive adjuvant mixed 1:1 with PBS in a final dose volume of 300 μl. Blood is collected from the tail vein periodically and analyzed for anti-HERV-K antibody immune response to the vaccine by evaluating binding of serial dilutions of serum to the peptide antigen coated on an ELISA plate. The isotypic profile of vaccine-induced antibodies (IgG1, IgG2a, IgG2b, IgG2c and IgM) is determined. This vaccination protocol produced high levels of IgG1 antibodies and a predominately Th2 immune response 65, 64 presumably induced by the Th2 promoting adjuvant, which would promote B cell activation and antibody production. Panels of CD4 and CD8 lymphocyte subsets are also evaluated to establish predictors of the IgG immune response 65. Mice are monitored for development of primary xenograft tumors and the experiment is terminated when the tumor volume reaches ˜2.0 cm³.

### Example 22

### HERV-K Blockade Leads to Increased Sensitivity to Chemotherapy and Therapeutic Drug Treatment

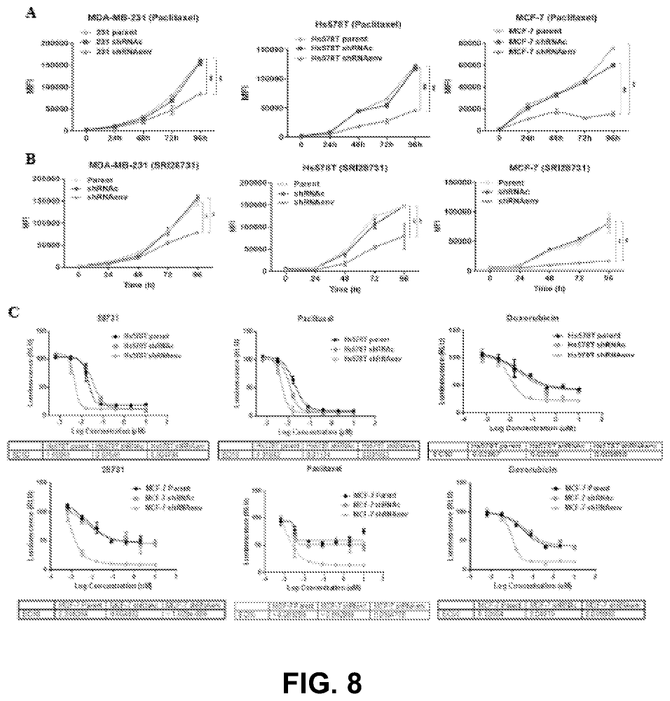

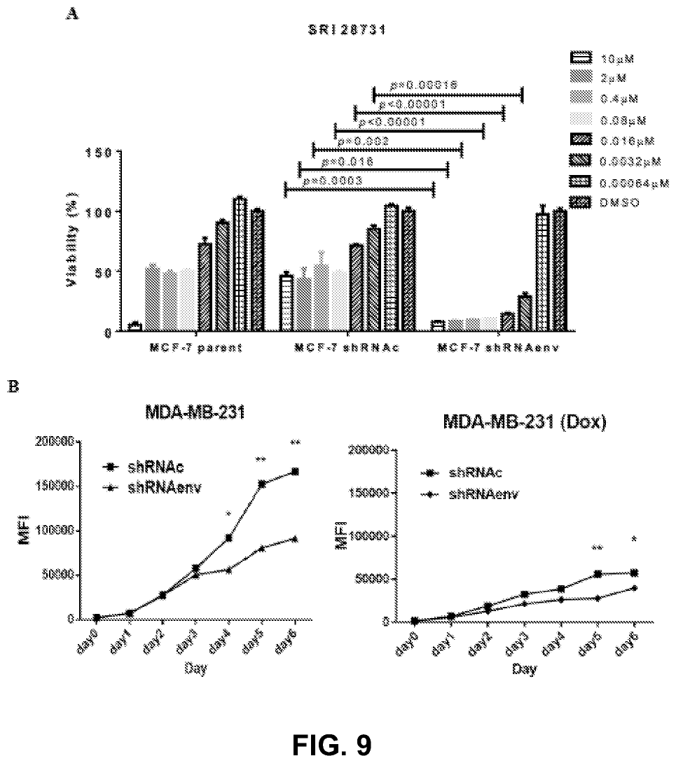

The inventors previously reported significantly reduced breast cancer cell proliferation after HERV-K KD with shRNAenv 10. Here, the inventors further determined the effects of HERV-K env gene KD in breast cancer cells treated with breast cancer chemotherapy drugs. Significantly reduced cell proliferation was observed in MDA-MB-231, Hs578T, and MCF-7 breast cancer cells treated with paclitaxel (FIG. 8A) or a highly potent and selective MAP4K4 (HGK) inhibitor SRI-28731 (FIG. 8B) if the HERV-K env gene was knocked down by stable transduction of shRNAenv, compared with their parent cells or shRNAc. Enhanced sensitivity of breast cancer cells toward SRI-28731, paclitaxel, and doxorubicin was demonstrated in MCF-7 and Hs578T cell lines (FIG. 8C) stably transduced with shRNAenv compared with cells transduced with shRNAc or parent cells. There was a decrease in EC50 by of at least 5-fold, compared to drug treatment alone or to cells that had been transduced with the scrambled control shRNAc. Reduced viability after KD of HERV-K was demonstrated in MCF-7 cells treated with SRI-28731 (FIG. 9A), and in MDA-MB-231 cells treated with doxorubicin (FIG. 9B), compared to their control cells (shRNAc). In summary, transduction of breast cancer cells with our shRNAenv inhibitor of HERV-K env mRNA showed synergy with standard of care therapy effects on cell proliferation and progression. Thus, the sensitivity of breast cancer cells toward anticancer agents was greatly increased by a factor of at least five after KD of HERV-K.

### Example 23

### HERV-K Presence Promotes Migration and Invasion of Breast Cancer Cells

The effects of KD of HERV-K env on migration and invasion were investigated using transwell plates. After transwell plates (8 μm) were rehydrated for 2 hours, 2.5×10⁴ cells were seeded into the transwell and cultured. The transwell was then removed and the cells that had migrated into the lower chamber were counted. For the invasion assay, transwells were coated with Matrigel, and cells remaining in the upper chamber were removed with a cotton swab. The invaded cells on the reverse side of the Matrigel were counted under a light microscope (40× magnification) after the cells were fixed with methanol and stained with Giemsa. Ten random fields were counted. The average number of invaded cells per field was presented as mean±SD (n=10 fields). Triplicate assays were performed for each experiment.

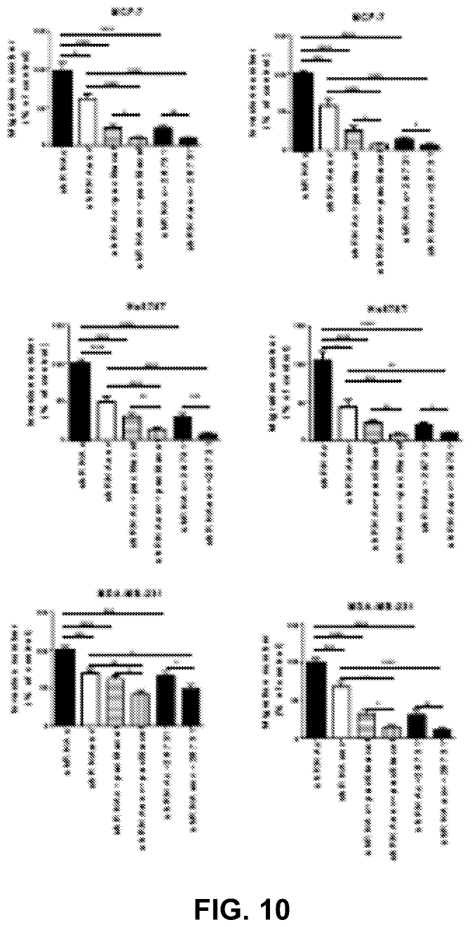

Significantly reduced migration and invasion was demonstrated in MCF-7 (FIG. 10A, HS578T cells (FIG. 25B, or MDA-MB-231 (FIG. 10C cells after treatment with paclitaxel or SRI-28731 (0.1 M), or after KD of HERV-K (FIG. 10D. Effects on migration and invasion were especially prominent in shRNAenv KD cells.

### Example 24

### HERV-K Knockdown Induces Cell Cycle Arrest

Cell cycle distribution was determined in breast cancer cell lines transduced with shRNAenv vs. shRNAc using FACS, S phase arrest was observed in MCF-7 and Hs578T breast cancer cell lines transduced with shRNAenv compared with control cells. In addition, G2 arrest was observed in the Hs578T cells treated with paclitaxel or SRI-28731, especially in the shRNAₑₙᵥ cells.

### Example 25

### Phosphoprotein Array and RNA-Seq Analyses Reveal Strong Effects of HERV-K Knockdown on Signal Transduction Pathways Relevant to Cancer

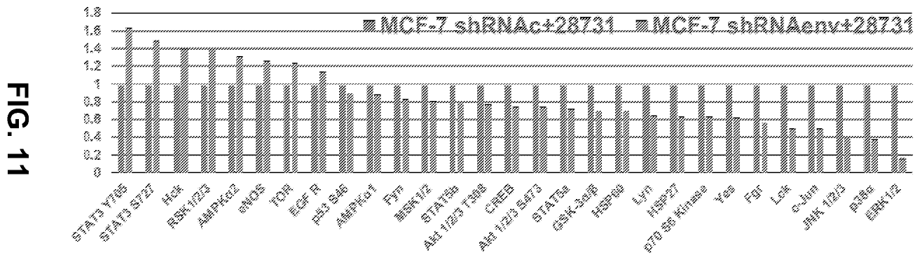

Phosphoprotein array analysis of MCF-7 cells transduced with shRNAenv or with shRNAc (blue) and treated with SRI-28731 revealed STAT3 Y705, STAT3 S727, Hck, RSK1/2/3, AMPKα2 as the five major upregulated proteins, and ERK1/2, p38×, JNK1/2/3, c-Jun, and Lck as the five major downregulated proteins after HERV-K KD cells were treated with SRI-28731. See FIG. 11.

The inventors' phosphoprotein array data support the concept that HERV-K expression in cancer activates two important signaling pathways: MAPK/Ras and P13K/AKT.

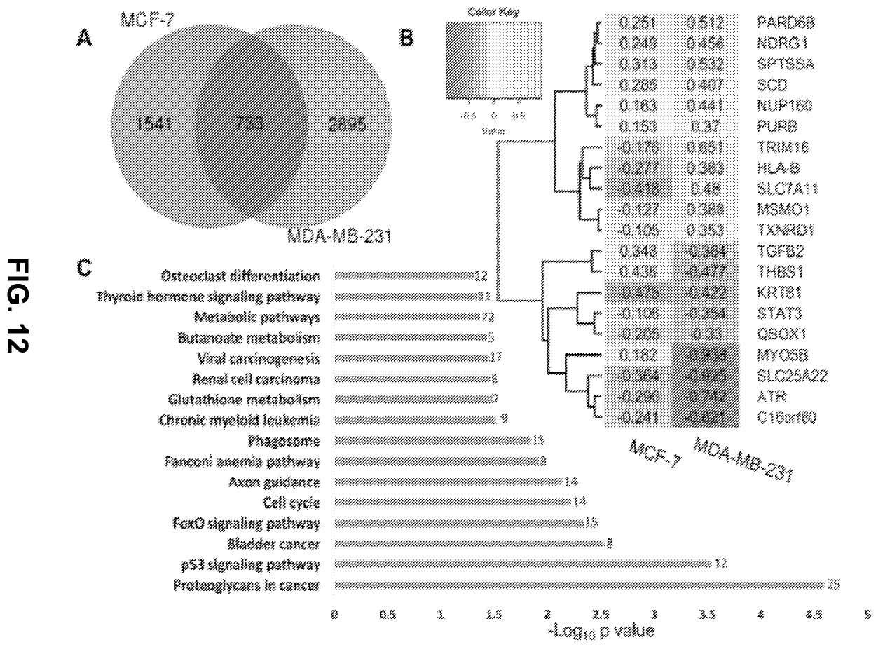

The inventors also used RNA-Seq data to determine differentially expressed genes in cancer cells after HERV-K KD. A Venn diagram of significant differentially expressed genes in MCF-7 and MDA-MB-231 after shRNA KD of HERV-K using RNA-Seq is shown in FIG. 12A and FIG. 12B. Clustering of the top 20 differentially expressed genes showed a divergence between the two cells after HERV-K KD by shRNA. Visualization and Integrated Discovery (DAVID) pathway analysis revealed the most differentially expressed classes to be proteoglycans in cancer (proteoglycans were recently shown to be critical for HERV-K entry into cells), p53 signaling pathway and others. See FIG. 12C. This is the first time that HERV-K expression was so closely associated with proteoglycans in cancer, indicating that HERV-K KD in cancer cells would have a very strong effect on expression of proteoglycans in these cells. In addition, all the proteins and pathways in this figure have the potential to be altered by the HERV-K status of the breast cancer cell.

### Example 26

### Increased Expression of HERV-K in Drug Resistant Breast Cancer Cell Lines

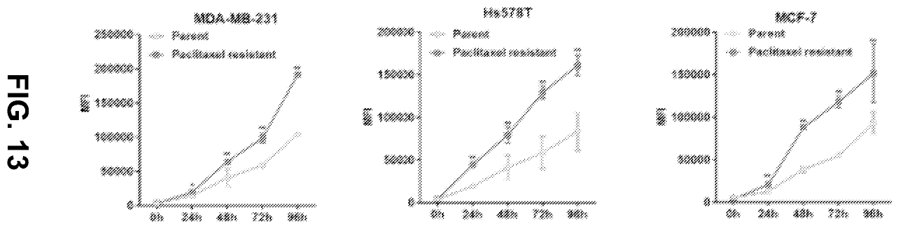

Enhanced expression of HERV-K was detected in three paclitaxel-resistant breast cancer cell lines (Tax) developed in our laboratory, compared with their parent cells (P) by RT-PCR (FIG. 13A) or FACS (FIG. 13B). Significantly increased cell proliferation was demonstrated in paclitaxel-resistant breast cancer cell lines (FIG. 13C).

### Example 27

Serum Levels of H₂O₂, Malondialdehyde, and Catalase Activity in Breast Cancer Patients Resistant to Chemotherapy

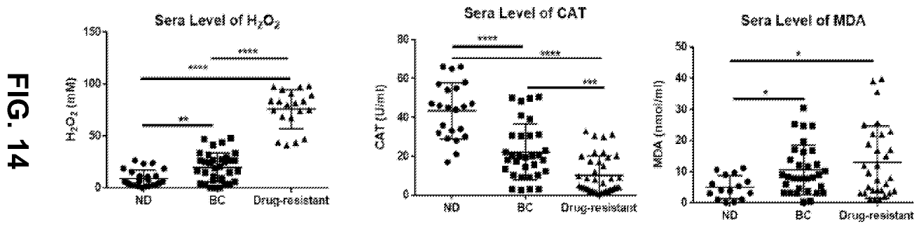

H₂O₂ is an endogenous reactive oxygen species. Serum levels of H₂O₂, the antioxidant catalase (CAT), and the marker of antioxidant damage malondialdehyde (MDA) were evaluated in patients with breast cancer (n=34) or paclitaxel-resistant breast cancer (n=20) compared with normal female donors (n=23; TABLE 7 and FIG. 14). Significantly increased serum levels of the reactive oxygen species H₂O₂ and malondialdehyde but decreased serum levels of catalase were observed in patients with breast cancer, especially in paclitaxel-resistant breast cancer patients.

TABLE 7
Characteristics of patients with ductal carcinoma

| Variable | Result (n = 78) | |
|---|---|---|
| Age (years) ± SD | 48.9 ± 13.4 | |
| Histological type | | |
| Ductal | 78 | (100%) |
| Stage, n (%) | | |
| I | 7 | (9%) |
| II | 26 | (33.3%) |
| III | 29 | (37.2%) |
| IV | 16 | (20.5%) |
| Grade | | |
| G1 | 11 | (14.1%) |
| G2 | 50 | (64.1%) |
| G3 | 17 | (21.8%) |
| lymph node positive | | |
| No | 52 | (66.7%) |
| Yes | 26 | (33.3%) |
| Lymphovascular invasion | | |
| No | 70 | (89.7%) |
| Yes | 8 | (10.3%) |
| HER2 | | |
| Negative | 59 | (75.6%) |
| Positive | 19 | (24.4%) |
| ER | | |
| negative | 29 | (37.1%) |
| positive | 49 | (62.8%) |

TABLE 7
Characteristics of patients with ductal carcinoma

| Variable | Result (n = 78) |
|---|---|
| Age (years) ± SD | 48.9 ± 13.4 |
| Histological type | |
| PR | |
| negative | 36 (46.9%) |
| positive | 42 (53.8%) |

### Example 28

### Reactive Oxygen Species Induces HERV-K Expression, Cancer Cell Proliferation, and Cancer Cell Migration

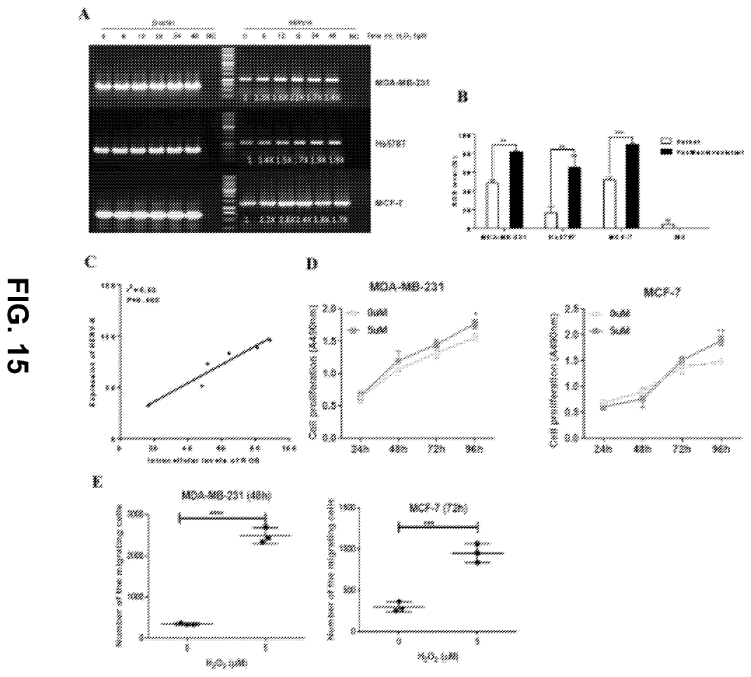

These findings led us to explore the relationship between the expression of HERV-K and reactive oxygen species concentration, with a finding of elevated expression of HERV-K mRNA in three breast cancer cell lines treated with H₂O₂ (5 M) at various exposure times (0 hours to 48 hours; FIG. 15A). Reactive oxygen species levels (FIG. 15B) were increased in the three paclitaxel-resistant breast cancer cell lines compared with their parent cells, and intracellular levels of ROS were positively associated with HERV-K expression, as assessed using Pearson's correlation (FIG. 15C). Significantly increased cell proliferation was observed after 96 hours in breast cancer cell lines treated with H₂O₂ (5 μM) (FIG. 15D) and increased migration was observed after 48 hours for MDA-MB-231 and after 72 hours for MCF-7 cells (FIG. 15E).

### Example 29

### Reactive Oxygen Species and Chemotherapeutic Agents Regulate the Expression of HERV-K, HIF-1α, P-RSK, P-ERK, and Ras

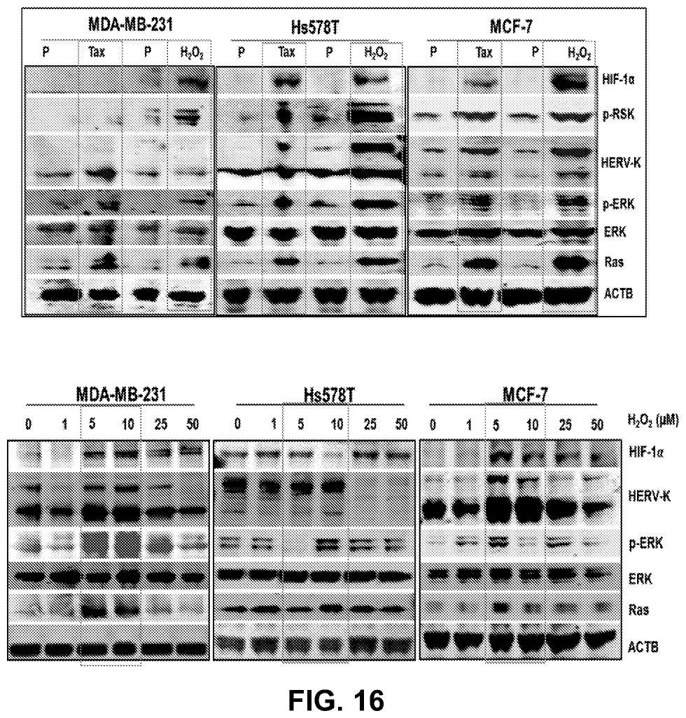

The inventors found increased expression of the breast cancer relevant signaling proteins HIF-1α, HERV-K, p-RSK, p-ERK, ERK, and Ras in breast cancer cells treated with paclitaxel (Tax, 0.1 μM; FIG. 16A) or H₂O₂ (5 μM or 10 μM; FIG. 16A and FIG. 16B) compared with their parent cells by immunoblot. Cells treated with graded concentrations of H₂O₂ ranging from 1-50 μM showed enhanced expression of HERV-K, Ras, p-ERK, and HIF-1a proteins at H₂O₂ concentrations of 5 μM and 10 μM in the three breast cancer cell lines (FIG. 16B).

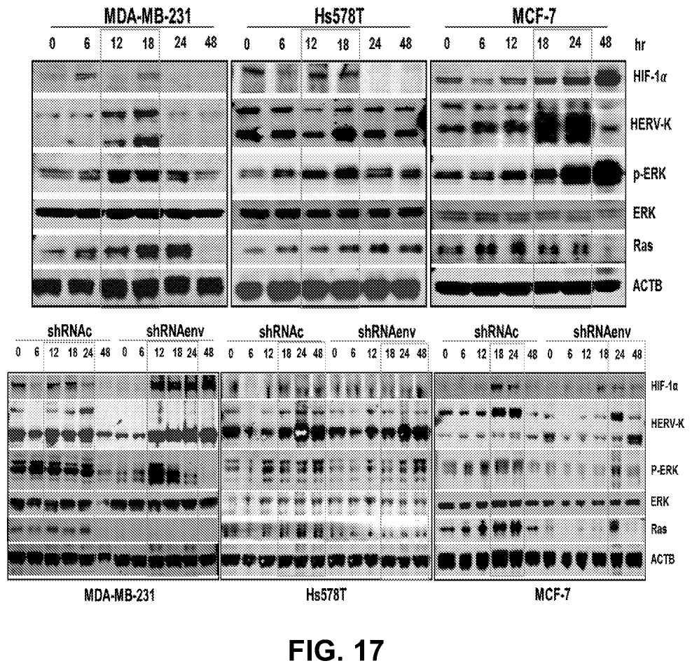

A time course study revealed increased expression of HERV-K and other signaling proteins in a time dependent manner, with peak expression at 12 hours to 18 hours in MDA-MB-231 and Hs578T TNBC cells, and 18 hours to 24 hours in MCF-7 breast cancer cells after treatment with 5 μM H₂O₂ (FIG. 17A). Low concentrations of H₂O₂ (5 μM) reversed the shRNAenv-induced block in expression of HERV-K, HIF-1α, P-RSK, and P-ERK in a time-dependent manner, apart from Ras expression, which remained refractory to H₂O₂ treatment (except for MCF-7 cells at 24 hours) (FIG. 17B).

These studies were confirmed in MDA-MB-231 and MCF-7 breast cancer cells by FACS analysis.

### Example 30

### Reactive Oxygen Species Increases Biomarkers of EMT Via Induction of HERV-K Expression

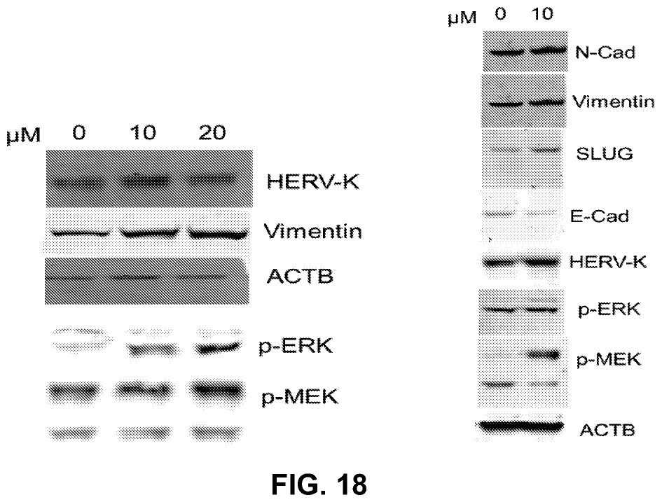

FACS analysis revealed expression of HERV-K and the EMT-associated proteins β-catenin and Slug in cells treated with H₂O₂ (10 μM). Immunoblots revealed enhanced expression of HERV-K, p-MEK, and p-ERK, as well as expression favoring EMT markers such as E-cadherin, N-cadherin, vimentin, and Slug in breast cancer cells treated with H₂O₂ for 18 hours (FIG. 18). Increased expression of p-MEK p-ERK, N-cadherin, vimentin and Slug and decreased expression of E-cadherin was correlated with enhanced expression of HERV-K.

### Example 31

### Significance of Reactive Oxygen Species/HERV-K/EMT Interactions

Enhanced expression of HERV-K Env protein was demonstrated in paclitaxel-resistant breast cancer cell lines and in cancer cells treated with H₂O₂. Of interest, enhanced expression of HIF-1α, p-RSK, p-ERK, and Ras was also observed. Increased expression of EMT markers including N-cadherin, vimentin, and Slug, and decreased expression of E-Cadherin was demonstrated in MDA-MB-231 cells treated with H₂O₂. These changes were associated with increased expression of HERV-K Env, p-ERK, and p-MEK. These data demonstrate that HERV-K is an upstream modulator of the Ras/ERK signaling pathway, and its expression is stimulated by physiological levels of reactive oxygen species. Thus, ROS (5 μM to 10 μM) upregulates the expression of HERV-K, and HERV-K in turn induces EMT. Collectively these data indicate that ROS and/or HERV-K inhibitors can blockade the EMT that initiates invasion and metastasis of cancer cells.

### Example 32

### HERV-K Exhibits Specific Anti-Tumor Cell Cytotoxicity in Drug-Resistant Breast Cancer

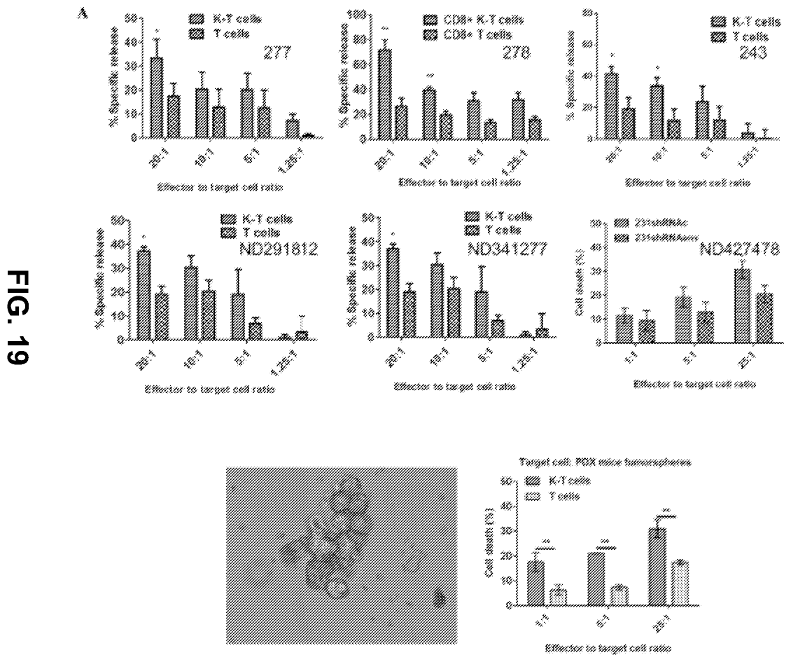

Anti-tumor effects were determined in drug-resistant breast cancer cells. A CTL assay was used to determine HERV-K specific cytotoxicity toward MDA-MB-231 cells using PBMCs obtained from breast cancer patients (patient #277 and 278 diagnosed with invasive ductal carcinoma (IDC), and #243 diagnosed with ductal carcinoma in situ (DCIS) or normal female donors (ND291812, ND341277, and ND427478). PBMCs were in vitro stimulated (IVS) with their autologous dendritic cells pulsed with KSU protein (K-T cells) for one week and cell death by target cell lysis was determined. See FIG. 19A. An enhanced percentage of target cell lysis was observed using CD8⁺ K-T effector cells, when compared with CD8⁺ T cells (patient #278, FIG. 19A top middle). There was also a decrease in lysis of target cells with HERV-K shRNAenv KD, compared to the shRNA control (ND427478, FIG. 19A bottom right), which reflects the specificity of K-T cells for the HERV-K target.

One PDX from tumor tissue of a TNBC patient diagnosed with IDC was generated and the expression of HERV-K was detected by immunohistochemistry (IHC) using 6H5 mAb. Mammospheres were cultured from the tumor tissue (FIG. 36B, left panel) and used as target cells for CTL assays (FIG. 19B, right panel). Significantly increased killing of the PDX mammosphere cells by K-T cells was demonstrated, compared with T cell killing.

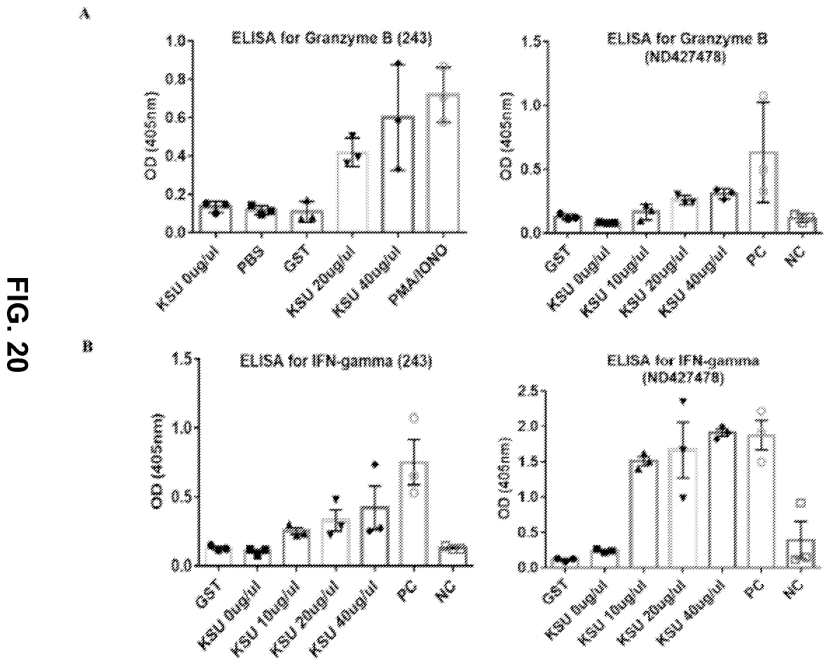

ELISA assays were used to detect secretion of granzyme B (FIG. 20A) and IFNγ (FIG. 20B) by IVS cells generated from PBMCs of a patient (#243) or a normal donor (ND427478). A greater release of IFNγ cytokine and granzyme B was detected with increased concentrations of KSU used to pulse IVS cells.

### Example 33

Administration of T Cells Pulsed with Dendritic Cells Loaded with HERV-K (K-T Cells) LED to Reduced Tumor Weights and Decreased Expression of Cell Signaling Pathway Intermediates that are Integral to the Formation and Growth of Cancer

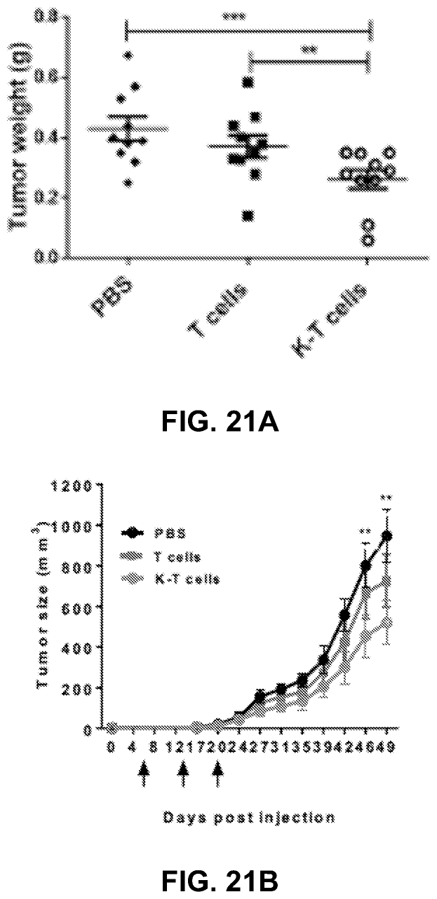

Mice were treated with PBS, T cells, or K-T cells on days 5, 13, and 21 post-inoculation with MDA-MB-231 cells. Significantly reduced tumor weights (FIG. 21A) and growth (FIG. 21B) were observed in mice treated with K-T cells than with T cells and/or PBS.

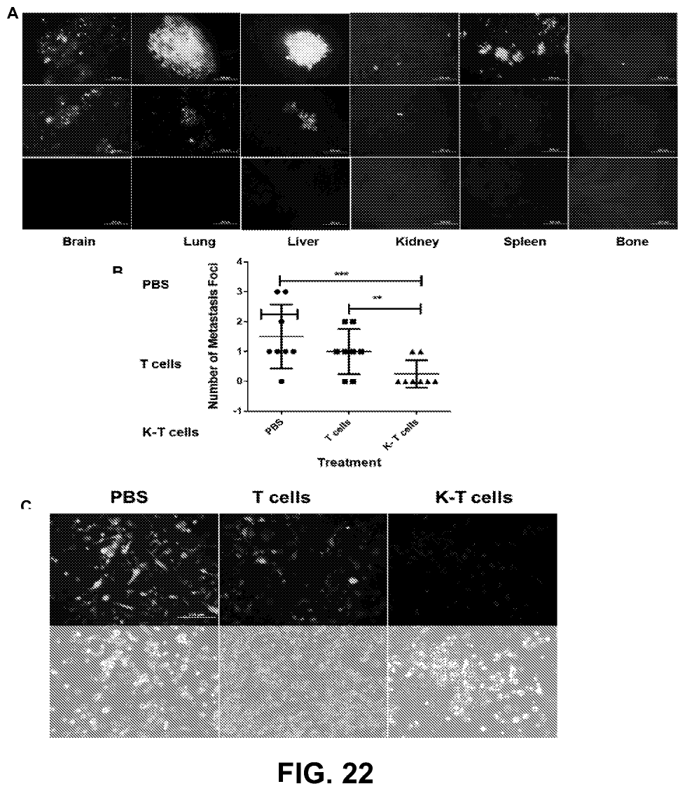

Metastatic cells (green color, FIG. 22A) in various organs including tumor, lung, liver, kidney, and brain were compared among various treatments and numbers of metastatic foci were determined. See FIG. 22B. Lung cells were cultured in RPMI, and metastatic cells (green fluorescence) were observed only in tissues from mice treated with T cells or PBS. See FIG. 22C.

The expression of HERV-K was evaluated in tumors and other organs by IHC or by FACS using 6H5 mAb. Significantly reduced expression of HERV-K Env protein was demonstrated in tumor or lung tissues of mice treated with K-T cells.

qRT-PCR was used to determine the expression of HERV-K, TP53, MDM2, and CDK5 using their specific primer pairs. Reduced expression of MDM2 or CDK5 and increased expression of P53 correlated with decreased expression of HERV-K in mice treated with K-T cells compared with other cell therapies. Decreased expression of HERV-K Env protein, MDM2, p-ERK and Ras was further demonstrated by immunoblot in tumor tissues of mice treated with K-T cells. CDK5, which plays a role in the development and progression of many human cancers, localizes in the mitochondria, a key determinant of apoptotic cell death. CDK5 loss increases chemotherapy-induced apoptosis.

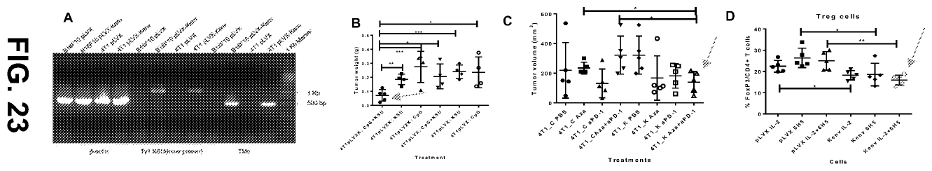

HERV-K/checkpoint blockade, HERV-K/DNA hypomethylation and HERV-K/interleukin combined therapy. Murine mammary tumor cells (4T1) or melanoma cells (B16F10) were engineered to express HERV-K Env34 to produce syngeneic models of HERV-K crucial for studying the role of the anti-tumor immune response. This was accomplished by stably transfecting cells with pLVXKenv [full length HERV-K env, expressing both extracellular surface (SU) a transmembrane (TM) domains] or pLVX vector only (control; FIG. 23A). Preliminary breast cancer data from our lab has indicated the potential for combination approaches with anti-HERV-K therapy including CpG with KSU (FIG. 23B), Aza combined with anti-PD-1 antibody (blue arrow: FIG. 23C) or 6H5 combined with IL-2 (blue arrow: FIG. 23D). Using this model, increased weight of pLVXKenv tumors relative to pLVX control cell tumors was observed in mice immunized with GST (2-fold increased weight). In contrast, the inventors observed reduced weight of pLVXKenv relative to pLVX tumors in mice immunized with KSU (50% reduced weight), showing the protective effect of KSU vaccination. This protective effect disappeared in mice immunized with the TM (1.65-fold increased tumor weight), indicating that the immunosuppressive domain (ISD) of TM62 may prevent an immune response to the vaccine.

### Example 34

### Induction of Immune Response in Human Cells:

The inventors tested for the presence of anti-HERV-K T-cell responses in human PBMCs from ovarian cancer patients and normal donors. We determined whether a CTL immune response can be elicited in ovarian cancer patients. DCs were generated from adherent PBMCs in cultures containing the cytokine combination of GM-CSF and IL-4. Immature DCs were pulsed with or without HERV-K proteins or cRNA and TNF-α for maturation. We compared the level of HERV-K-specific T-cell responses in normal female donors to ovarian cancer patients using in vitro stimulated (IVS) PBMC. The DCs were pulsed with HERV-K cRNA and mixed with autologous PBMC for 7 days to generate singly stimulated IVS cells. HERV-K-specific T-cell proliferation and CTL activity was determined, using INFγ ELISPOT.

CD4+ T cell proliferation was compared in freshly isolated (ex vivo) PBMC versus IVS cells pulsed with HERV-K SU protein from two ovarian cancer patients (#810806 and #807218) and two patients with fibrous adhesions and cyctic benign serous cyst, no malignancy identified (#811578) and benign serous cystadenoma (#819581). Marked HERV-K-specific proliferation was detected after IVS of PBMC from OC patients (#810806 and #807218), with a significant difference between IVS generated by DC pulsed with HERV-K SU env protein (DC+K10) than by DC pulsed with HPV 16 E6 protein (DC+E6, as control). No HERV-K-specific proliferation was detected after IVS of PBMC from a patient with benign serous cyst (#810806) and benign serous cystadenoma (#807218). Proliferation was greater in IVS obtained from OC patients than in IVS obtained from control subjects, which indicates that HERV-K env protein capable of inducing a CD4⁺ T cell response in OC.

### Example 35

HERV-K Protein Stimulation of Breast Cancer Patient PBMCs In Vitro with and without Checkpoint Blockade.

MCF7 cells were co-incubated for one week with T cells in PBMC from invasive ductal carcinoma patients BC351 and BC373 that were pulsed with KSU, transmembrane proteins TMC or TMV, or GST. The percentage for MCF7 lysis after co-incubation was greatest for both patients when T cells were pulsed with KSU or TMV and was further increased in cells treated with anti-PD-L1 antibody. The pulsed TMV transmembrane variant showed greater efficacy in cancer cell killing than the pulsed TMC consensus TM sequence

PBMCs from 3 breast cancer patients and 1 normal donor were pulsed with the HERV-K proteins KSU, TMC, or TMV. HERV-K stimulated PBMCs from these subjects showed increased percentages of CD3, CD4, and CD8, as well as exhaustion markers PD-1, CTLA-4 and LAG3, with the LAG3 plus either CD4 or CD8 showing especially large increase in the breast cancer patients.

The induction of a cancer-specific CD4+ T cell response in both breast and ovarian cancers, after pulsing T cells or PBMCs with HERV-K reflects a unique ability of this viral target to promote an innate immune response in multiple cancers.

### Example 36

### HERV-K SU/TM Specific T-Cell Responses in Human Breast Cancer Patients and Healthy Donors by Anti-Human IFN-γ ELISPOT Assay

T cell responses against the HERV-K SU and TM domain induced in breast cancer patients was evaluated by isolating PBMCs from the blood of breast cancer patients and corresponding healthy female donors. PBMCs, IVS-SU, and IVS-TM cells (5×10⁴ cells per well) from two patients with breast cancer and two healthy donors were co-cultured with autologous protein-pulsed DCs for 18-24 hrs. All samples were tested in triplicate. After in vitro stimulation of PBMCs by co-culturing with autologous DCs pulsed with HERV-K TM fusion protein for 1 week in the presence of rhIL-2, an antihuman IFN-γ ELISPOT assay was performed with the two breast cancer patients and two healthy donors. For the breast cancer patient samples, a significantly increased (P<0.05) number of spots was observed in the wells with IVS TM and IVS SU cells restimulated with autologous DCs pulsed with HERV-K TM protein compared to those co-cultured with the DCs pulsed with control KLH protein. In contrast, in the two healthy donors, an increase in the number of IFN-γ secreting cells was not observed in the IVS/TM cells co-cultured with TM-pulsed DCs, compared with numbers of the IVS/TM cells with KLH-pulsed DCs. Furthermore, a much higher IFN-γ response was induced after IVS using autologous TM-pulsed DCs in the two patient samples than in the two normal donors. The similar increased IFNγ production was evident in the IVS/SU cells re-stimulated by protein pulsed DCs in the two cancer patients compared to the two healthy individuals.

### Example 37

### Humoral and Cellular Immune Responses Against TM Protein in Mice

To investigate whether the TM fusion protein could induce a humoral TM-targeted immune response in mice, ELISA assays were used to analyze the antibody production by mice immunized with the recombinant TM fusion protein. The HERV-K TM fusion protein was produced in E. coli and purified. HLA-A2 transgenic mice were immunized subcutaneously followed by three boosts at 1-week intervals. Mice mock injected with PBS were used as a control. Sera were collected 10 days after the last boost and a serial dilution was prepared for testing the anti-TM antibodies by ELISA using TM fusion protein as the antigen. The HERV-K TM fusion protein was able to induce much higher TM-specific IgG titers (p<0.0001) in all the immunized mice (n=3) than in the mock-immunized mice (n=3).

Spleen cells were isolated from mice immunized with HERV-K TM protein or were mock-immunized (PBS) and then re-stimulated with TM protein (20 μg/mL) in ELISPOT plates (5×10⁵ cells per well) for 18-24 hours prior to spot development and counting. Data are presented as IFN-γ secreting spleen cells per half million spleen cells in TM-stimulated samples minus that measured in KLH samples. Significantly more IFN-γ secreting spleen cells were detected in the immunized mice than in the mock mice (P<0.05).

### Example 38

### ELISPOT Assay of IFN-γ-Secreting T Cells in Breast Cancer Patients and Healthy Control Donors

ELISPOT assays tested the HERV-K SU and TM specific T cell responses in 21 patients with breast cancer as well as 12 healthy normal donors. IFN-γ secreting IVS cells were co-cultured with either KLH-pulsed DCs or with DCs pulsed with HERV-K TM or SU protein. The IFNγ ELISPOT data revealed that, compared to the healthy donors, the number of IFNγ secreting cells from the breast cancer patient samples was significantly greater in IVS TM cells as well as IVS SU cells (FIG. 42(ii)B) after re-stimulation with autologous DCs pulsed with the corresponding proteins. The data suggest that specific T cell responses against both TM and SU protein were induced in breast cancer patients.

### Example 39

### Overexpression of the HERV-K Env Gene Activate Unmutated Ras Expression and the Ras/Raf/MEK/ERK Signaling Pathway in Cancer Cells

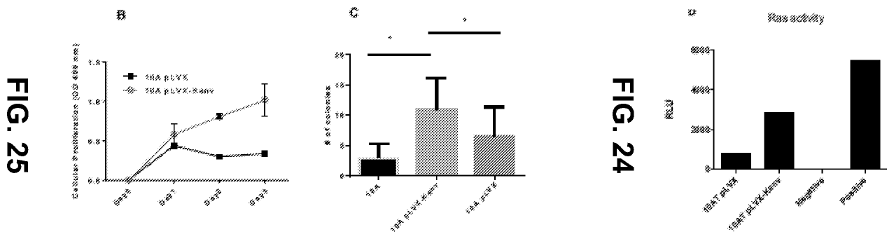

Effects on Ras expression in breast cancer cells transduced with shRNAenv to knock down the endogenous retrovirus HERV-K were compared with effects in cells transduced with a control shRNAc. KD of HERV-K led to decreased expression of oncogenic K-Ras, N-Ras, and H-Ras. See FIG. 24A and FIG. 24B. When the expression of HERV-K was restored (pLVXKenv, FIG. 24A and Kenv, FIG. 24B), reactivation of expression of Ras (FIG. 24A) and other proteins in the Ras/Raf/MEK/ERK pathway (FIG. 24B) was demonstrated. Overexpression of HERV-K in immortalized but non-tumorigenic MCF-10AT (FIG. 24) or MCF-10A (FIG. 24) breast cells resulted in increased expression of K-Ras and N-Ras (FIG. 24C or FIG. 25A) and increased Ras activation (FIG. 24D) as well as transformation in MCF-10A+pLVXKenv cells. Of interest, no RAS mutations in K-RAS, N-RAS, or H-RAS were detected by sequencing in MCF-10AT cells stably transfected with pLVXKenv. The significance of these findings to breast cancer is that the three Ras genes in humans, which are key molecular regulators controlling cell proliferation, transformation, differentiation, and survival are activated by HERV-K using a mechanism not involving mutational activation of Ras.

These results showed enhanced expression of HERV-K in three breast cancer cell lines that developed paclitaxel-resistance in their lab, or that were treated with the ROS hydrogen peroxide (H₂O₂), when compared with their parent cells by RT-PCR or Western blot. Overexpression of HIF-1α, HERV-K Env protein, p-RSK, p-ERK, and Ras were also discovered in paclitaxel-resistant or H₂O₂-treated breast cancer cells. The significance of these studies is that HIF-1×, a key transcription factor activated by ROS, and whose expression increases in breast cancer and indicates poor patient prognosis, is upregulated in tandem with HERV-K and Ras signaling pathway intermediates in several breast cancer cell lines. These results thus show that blocking HIF-1α expression via HERV-K KD can be an avenue for cancer therapy.

### Example 40

### Combination Therapy

The inventors' breast cancer data from strongly support the potential for combination therapy approaches involving HERV-K. Humanized and fully human antibodies targeting HERV-K will therefore enhance checkpoint blockade antibody treatment efficacy. Effective combined cancer therapies include but are not limited to combinations of (a) HERV-K hTAb (1.5 mg/kg), (b) K-CAR, (c) K-BiTE, (d) HERV-K shRNAs or CRISPR/Cas9 genome editing technology to knock down HERV-K gene expression, (e) or preventative or therapeutic HERV-K vaccines, including full-length and truncated HERV-K Env proteins and HERV-K Env peptides, and (a) anti-ICP antibody, (b) cancer chemotherapy, (c) 5-azacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion, (f) induction of S or G2 phase cell cycle arrest, (g) inhibitors of PI3K/AKT/mTOR or MAPK/ERK signaling pathways, or (h) signal transduction to HIF1a.

### Example 41

Envelope (Env), Gag, and Pol HERV-K RNA Sequences of a Viral Particle Isolated from a Breast Cancer Patient.

Viral peptide sequences were obtained from breast cancer patients.

### LIST OF EMBODIMENTS

Specific compositions and methods of HERV-K antibody therapeutics. The scope of the invention should be defined solely by the claims. A person having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

Among the embodiments provided in this specification are the following:
1. An isolated antibody that binds to human endogenous retrovirus-K (HERV-K), comprising a heavy chain variable region (HCVR) and a light chain variable region (LCVR). Humanized anti-HERV-K antibody is able reduce tumor growth, especially reduce metastasis to lung, lymph nodes and other organs.
2. The antibody, comprising a humanized or human framework region.
3. The antibody, wherein the antibody is an HERV-K antagonist.
4. An isolated nucleic acid comprising a nucleotide sequence encoding the HCVR, the LCVR, or a combination thereof.
5. An expression vector comprising the nucleic acid.
6. A host cell transformed with an expression vector.
7. A method of producing an antibody comprising a HCVR, a LCVR, or a combination thereof, the method comprising: growing the host cell, under conditions such that the host cell expresses the antibody comprising the HCVR, the LCVR, or a combination thereof; and isolating the antibody comprising the HCVR, the LCVR, or combination thereof.
10. A method of treating cancer in a mammal, comprising administering an effective amount of the antibody to a mammal in need thereof.
11. A method for treating cancer comprising administering, to an individual in need thereof, an effective amount of an ADC comprising an antibody comprising a heavy chain variable region (VH) and a light chain variable region (VL), wherein: the VH region comprises a CDR1, a CDR2, and a CDR3, and the VL region comprises a CDR1, a CDR2, and a CDR3, wherein the antibody is conjugated to a cytotoxic drug, an auristatin or a functional peptide analog or derivate thereof via a linker.
12. The method of embodiment 11, wherein the ADC is administered in combination with one or more additional therapeutic agents.
13. The method of embodiment 11, wherein the one or more additional therapeutic agents includes a chemotherapeutic agent.
14. The method of embodiment 11, wherein the cancer is selected from the group consisting of melanoma, chronic lymphocytic leukemia, breast cancer, pancreatic cancer, head and neck cancer, ovarian cancer, cervical cancer, colorectal cancer, testicular cancer, stomach cancer, kidney cancer, endometrial cancer, uterine cancer, bladder cancer, prostate cancer, esophageal cancer, liver cancer, and non-small cell lung cancer.
15. The humanized antibody developed for CAR T, CAR NK, and BITE studies.
16. The method of embodiment 11, wherein the antibody is a full-length antibody.
17. The method of embodiment 11, wherein the antibody is a human monoclonal IgG1 or IgG4 antibody.
18. The method of embodiment 11, wherein the auristatin is monomethyl auristatin E (MMAE).
19. The method of embodiment 11, wherein the auristatin is monomethyl auristatin F (MMAF).
20. The method of embodiment 11, wherein the cytotoxic drug is emtansine (DM1).
21. The method of embodiment 11, wherein the cytotoxic drug is ozagamicin (calicheamicin).
22. The method of embodiment 11, wherein the cytotoxic drug is deruxtecan (DXd).
23. The method of embodiment 11, wherein the cytotoxic drug is govitecan (SN-38).
24. The method of embodiment 11, wherein the cytotoxic drug is mafodotin (MMAF).
25. The method of embodiment 11, wherein the cytotoxic drug is duocarmazine (duocarmycin).
26. The method of embodiment 11, wherein the cytotoxic drug is BAT8001 (maytansinoid) soravtansine (DM4).
27. The method of embodiment 11, wherein the cytotoxic drug is tesirine (PBD).
28. The method of embodiment 11, wherein the linker is attached to sulphydryl residues of the antibody obtained by partial reduction of the antibody.
29. The method of embodiment 11, wherein the linker-auristatin is vcMMAF or vcMMAE.
30. The early detection, metastasis, or HERV-K plus immune checkpoint biomarkers, substantially as described herein.
31. Antibody-based therapeutics, substantially as described herein.
32. Cancer cells overexpressing HERV-K as targets for the anti-HERV-K humanized antibodies and ADCs of the invention.
33. The hu6H5 clones (FWJ1 and FWJ2) generated from bacteria (HUM1 and HUM2) or mammalian cells.
34. A BiTE directed against T cell CD3 or CD8 and a humanized scFv against the tumor-associated antigen HERV-K, comprising antibodies targeting either CD3 or CD8 and HERV-K.
35. T cells expressing a lentiviral CAR expression vector that bears a humanized or fully human HERV-K scFv.
36. A humanized single chain variable fragment (scFv) antibody able to bind antigens produced from recombinant HERV-K Env surface fusion protein (KSU) and lysates from cancer cells expressed HERV-K Env proteins.
37. A CAR produced from the humanized scFv of embodiment 28.
38. A CAR produced from the humanized scFv of embodiment 28, which is cloned into a lentiviral vector.
39. A CAR produced from the humanized scFv of embodiment 28, which is cloned into a lentiviral vector, for use in combination therapies.
40. An improved in vivo enrichment method for rapid expansion and B cell activation for donors who have no memory B cells, comprising the steps of: treating mice with cytokine cocktails on days 1, 7, and 14, and boosting the mice by antigens on days 14 and 21.
41. Cells that not only produce antibodies, but the antibodies are also able to bind antigen and kill cancer cells, and cells that express the antigen are able to be killed by the antibodies.
42. A significantly enhanced expression of six circulating immune checkpoint proteins in the plasma of breast cancer patients.
43. A method of blockading of the immunosuppressive domain (ISD) with immune checkpoint inhibitors of HERV-K.
44. The method of embodiment 42, wherein the immune checkpoint inhibitors of HERV-K are selected from the group consisting of monoclonal antibodies and drugs targeting the ISD of HERV-K.
45. Humanized and fully human antibodies targeting HERV-K, for use in enhancing checkpoint blockade antibody treatment efficacy.
46. A method to produce new antibodies from mice immunized with 5 multiple antigen peptides (MAPs) that are generated from HERV-K SU protein produced by cancer patients.
47. A method to produce to produce HERV-K CAR A: VH-VLhu6H5-CD8-CD28-4-1BB-CD3zeta.
48. Full length HERV-K genes with open reading frames of gag, pol, and env in cancer patient blood and tissues.
49. Full length HERV-K envelope (ENV) and surface (SU) proteins in invasive cancer patient sera and tissues, but not in sera and tissues of normal females.
50. Enhanced reverse transcriptase (RT) activities in cancer patients, relative to activities in benign or normal female donors without cancer.
51. The HERV-K env gene isolated from a viral particle promotes tumor development and metastasis, in vitro and in vivo.
52. The early detection, metastasis, or HERV-K plus immune checkpoint biomarkers, substantially as described herein.
53. Anticancer vaccines, substantially as described herein.
54. Unique syngeneic models of HERV-K and human tumor mouse (HTM) models that can be immunized with HERV-K Env protein.
55. TCR sequences generated from TILs that recognize HERV-K antigens as peptides bound to the Major Histocompatibility Complex (MHC), resulting in an interaction between the HLA-peptide complex and the CD8 or CD3 TCR.
56. The combination of checkpoint inhibition and HERV-K therapies that include antibodies, T cell receptors (TCRs), vaccines, peptides, shRNAs, and other drugs, resulting in better cancer cell killing efficacy.
57. A platform enabling functional matching TCR sequences, which is acquired from a small number of homogenous HERV-K specific T cell (K-T cell) populations from a single clonally expanded K-T cell.
58. HERV-K specific T cells (K-T cells) from tumor infiltrating lymphocytes or peripheral blood mononuclear cells exhibiting secretion of IFNγ.
59. The use of HERV-K as a stem cell marker.
60. A method for the overexpression of HERV-K, comprising the steps of: administering cancer cells with agents that induce expression of HERV-K by innate immune response (Poly I:C treatment) or LTR hypomethylation (5-Aza), wherein the administration provokes the cancer cells to increase production of a target that makes the cancer cells more susceptible to targeted therapy to include targeted immunotherapy.
61. A platform to determine the binding kinetics and cell-to-cell interactions of every cell in a microwell slab.
62. A significantly enhanced expression of six circulating immune checkpoint proteins in the plasma of breast cancer patients.
63. Dendritic cells that were transfected with HERV-K surface (SU) envelope (Env) protein.
64. The use of the immunosuppressive domain (ISD) of HERV-K as an as an immune checkpoint on cancer cells.
65. A method of blockading of the immunosuppressive domain (ISD) with immune checkpoint inhibitors of HERV-K.
66. The method of claim 42, wherein the immune checkpoint inhibitors of HERV-K are selected from the group consisting of monoclonal antibodies and drugs targeting the ISD of HERV-K.
67. The use of reactive oxygen species to induce HERV-K expression, cancer cell proliferation, and cancer cell migration.
68. Blockade of reactive oxygen species signaling by inhibition of HERV-K expression.
69. A breast cancer cell line that has been treated with H₂O₂ intracellular levels of reactive oxygen species that are positively associated with HERV-K expression.
70. A combination of reactive oxygen species and chemotherapeutic agents to regulate the expression of HERV-K, HIF-1α, P-RSK, P-ERK, and Ras.
71. Cells treated with graded concentrations of H₂O₂ ranging from 1-50 μM showed enhanced expression of HERV-K, Ras, p-ERK, and HIF-1a proteins at H₂O₂ concentrations of 5 M and 10 μM in the three breast cancer cell lines.
72. The use of reactive oxygen species to increase biomarkers of EMT via induction of HERV-K expression.
73. Inhibition of HERV-K expression to decrease reactive oxygen species-mediated induction of EMT.
74. The HERV-K env gene promotes expression of multiple oncogenes including Ras (especially KRas), p-ERK, c-myc, HIF-1alpha, and AMPK beta; and downregulates expression of caspases 3 and 9, p-RB, CIDEA, p-P38, eNOS, and AMPK alpha.
75. The HERV-K env gene promotes expression of multiple oncogenes including Ras (especially KRas), p-ERK, c-myc, HIF-1alpha, and AMPK beta; and downregulates expression of caspases 3 and 9, p-RB, CIDEA, p-P38, eNOS, and AMPK alpha.
76. The use of HERV-K as an upstream modulator of the Ras/ERK signaling pathway.
77. Peripheral blood mononuclear cells that have been in vitro stimulated with their autologous dendritic cells pulsed with KSU protein (K-T cells).
78. A method for evaluating the expression of HERV-K in tumors and other organs by immunohistochemistry or by FACS using 6H5 mAb.
79. The use of the three Ras genes in humans, when activated by HERV-K using a mechanism not involving mutational activation of Ras.
80. Decreased activity of wild-type (non-mutated) and mutated Ras and Ras-associated signaling pathways by inhibition of HERV-K expression.
81. Sequences of HERV-K Envelope (Env), Gag, and Pol genes and RNAs extracted from viral particles isolated from breast cancer patients.
82. Viral particles and the oncogene of Kenv isolated from the viral particles.

### REFERENCES

A person having ordinary skill in the molecular biological art of can use the following patents, patent applications, and scientific references as guidance to predictable results when making and using the invention.

### Patent References

U.S. Pat. No. 9,243,055 (Wang-Johanning). This patent discloses and claims cancer diagnostics and therapy. Methods and compositions for detecting, preventing, and treating HERV-K+ cancers are provided. One method is for preventing or inhibiting cancer cell proliferation by administering to a subject a cancer cell proliferation blocking or reducing amount of a HERV-K env protein binding antibody.
International Pat. Publ. WO 2010/138803 (Board of Regents, the University of Texas System) discloses an isolated antibody that hinds to human endogenous retrovirus-K (HERV-K), comprising a heavy chain variable region (HCVR) and a light chain variable region (LCVR) (HERV-K protein recognized by an antibody, with a light chain variable region and a heavy chain variable region.
International Pat. Publ. WO 2014/186469 (Board of Regents, the University of Texas System). This patent publication concerns methods and compositions for immunotherapy using a modified T cell comprising a chimeric antigen receptor (CAR). CAR-expressing T-cells are produced using electroporation in conjunction with a transposon-based integration system to produce a population of CAR-expressing cells that require minimal ex vivo expansion or that can be directly administered to patients for cancer treatment.
International Pat. Publ. WO 2019/104037 A1 (The Brigham and Women's Hospital, Inc.)

### Non-Patent References

Burstein & Winer, Refining therapy for human epidermal growth factor receptor 2-positive breast cancer: T stands for trastuzumab, tumor size, and treatment strategy. J. Clinical Oncol., 27, 5671-5673 (2009).
Buscher et al., Expression of human endogenous retrovirus K in melanomas and melanoma cell lines. Cancer Research, 65, 4172-4180 (2005).
Cao, Human endogenous retroviruses in clear cell renal cell carcinoma: biological functions and clinical values.” Duvtlμress Web (Aug. 7, 2020).
Chae et al., Epithelial-mesenchymal transition (EMT) signature is inversely associated with T-cell infiltration in non-small cell lung cancer (NSCLC). Science Reports, 8, 2918 (2018).
Chiappinelli et al., Inhibiting DNA methylation causes an interferon response in cancer via dsRNA including endogenous retroviruses. Cell, 16, 974-986 (2015).
Choi et al., Human B cell development and antibody production in humanized NOD/SCID/IL-2Rgamma (null) (NSG) mice conditioned by busulfan. J. Clinical Immunol., 31, 253-264 (2011).
Denne et al., Physical and functional interactions of human endogenous retrovirus proteins Np9 and rec with the promyelocytic leukemia zinc finger protein. J Virol., 81, 5607-5616 (2007).
Downey et al., Human endogenous retrovirus K and cancer: Innocent bystander or tumorigenic accomplice? Int. J. Cancer, 137, 1249-1257 (2015).
Ejthadi et al., A novel multiplex RT-PCR system detects human endogenous retrovirus-K in breast cancer. Arch Virol. 2005; 150:177-184.
Elgueta et al., Molecular mechanism and function of CD40/CD40L engagement in the immune system. Immunol. Rev., 229, 152-172 (2009).
Etkind, Lumb, Du, & Racevskis, Type 1 HERV-K genome is spliced into subgenomic transcripts in the human breast tumor cell line T47D. Virology. 1997; 234:304-308.
Ettinger et al., IL-21 induces differentiation of human naive and memory B cells into antibody-secreting plasma cells. J Immunol. 2005; 175:7867-7879.
Feldhaus et al., Flow-cytometric isolation of human antibodies from a nonimmune Saccharomyces cerevisiae surface display library. Nature Biotechnol. 2003; 21:163-170.
Fischer et al., Human endogenous retrovirus np9 gene is over expressed in chronic lymphocytic leukemia patients. Leuk. Res. Rep., 3, 70-72 (2014).
Gonzalez-Cao et al., Human endogenous retroviruses and cancer. Cancer Biol Med., 13, 483-488 (2016).
Good, Bryant, & Tangye, Kinetics of human B cell behavior and amplification of proliferative responses following stimulation with IL-21. J. Immunol., 177, 5236-5247 (2006).
Grandi, HERV envelope proteins: Physiological role and pathogenic potential in cancer and autoimmunity. Frontiers in Microbiology. Web (Mar. 14, 2018).
Helsen et al., The chimeric TAC receptor co-opts the T cell receptor yielding robust anti-tumor activity without toxicity. Nature Commun. 2018; 9:3049.
Herve et al., Autoantibodies to human endogenous retrovirus-Kare frequently detected in health and disease and react with multiple epitopes (Apr. 28, 2002) discloses antibodies specific to HERV-K (autoantibodies specific to HERV-K).
Hipp et al., IL-2 imprints human naive B cell fate towards plasma cell through ERK/ELK1-mediated BACH2 repression. Nature Commun., 8, 1443 (2017).
Hughes & Coffin, Evidence for genomic rearrangements mediated by human endogenous retroviruses during primate evolution. Nature Genetics, 29, 487-489 (2001). HERVs originated from thousands of ancient integration events which incorporated retrovirus DNA into germline cells.
Johanning et al., Expression of human endogenous retrovirus-K is strongly associated with the basal-like breast cancer phenotype. Sci. Rep., 7, 41960 (2017). The inventors observed the expression of HERVs, especially HERV-K sequences, in breast, lung, prostate, ovarian, colon, pancreatic, and other solid tumors. They also found that the expression of HERV-K env transcripts in breast cancer was specifically associated with basal breast cancer, a particularly aggressive subtype.
Kleiman et al., HERV-K (HML-2) GAG/ENV antibodies as indicator for therapy effect in patients with germ cell tumors. Int J Cancer, 110, 459-461 (2004).
Koncz & Hueber, The Fas/CD95 receptor regulates the death of autoreactive B cells and the selection of antigen-specific B cells. Front. Immunol., 3, 207 (2012).
Kontermann & Brinkmann, Bispecific antibodies. Drug Discovery Today (2015).
Kontsekova et al., First-in-man tau vaccine targeting structural determinants essential for pathological tau-tau interaction reduces tau oligomerisation and neurofibrillary degeneration in an Alzheimer's disease model. Alzheimers Res Ther., 6, 44 (2014).
Kraus et al., Vaccination directed against the human endogenous retrovirus-K envelope protein inhibits tumor growth in a murine model system. PLOS One, 8, e72756 (2013).
Krishnamurthy et al., Genetic Engineering of T Cells to Target HERV-K, an Ancient Retrovirus on Melanoma. Clin Cancer Research 2015; 21:3241-3251. The inventors observed the expression of HERVs, especially HERV-K sequences, in breast, lung, prostate, ovarian, colon, pancreatic, and other solid tumors.
Lander et al., Initial sequencing and analysis of the human genome. Nature, 409, 860-921 (2001). HERVs are well-known as genomic repeat sequences, with many copies in the genome, such that approximately 8% of the human genome is of retroviral origin.
Lanzavecchia, Corti, & Sallusto, Human monoclonal antibodies by immortalization of memory B cells. Current Opinion Biotechnol., 18, 523-528 (2007).
Larsson Kato & Cohen, Human endogenous proviruses. Current Topics Microbiol. Immunol., 148, 115-132 (1989). The most biologically active HERVs are members of the HERV-K family. HERV-K has a complete sequence capable of expressing all the elements needed for a replication-competent retrovirus
Larsson, Kato, & Cohen, Human endogenous proviruses. Current Topics Microbiol. Immunol.; 148, 115-132 (1989). The most biologically active HERVs are members of the HERV-K family. HERV-K has a complete sequence capable of expressing all the elements needed for a replication-competent retrovirus.
Lemaitre et al., A human endogenous retrovirus-derived gene that can contribute to oncogenesis by activating the ERK pathway and inducing migration and invasion. PLOS Pathog., 13, e1006451 (2017).
Li et al., Down-regulation of human endogenous retrovirus type K (HERV-K) viral env RNA in pancreatic cancer cells decreases cell proliferation and tumor growth. Clinical Cancer Research, 23, 5892-5911 (2017). The inventors observed the expression of HERVs, especially HERV-K sequences, in breast, lung, prostate, ovarian, colon, pancreatic, and other solid tumors.
Liu et al., Structure-based programming of lymph-node targeting in molecular vaccines. Nature, 507, 519-522 (2014).
Maldini, Dual CD4-based CART cells with distinct costimulatory domains mitigate HIV pathogenesis in vivo 1. Nature Medicine Web (Aug. 31, 2020).
Mazor et al., Isolation of engineered, full-length antibodies from libraries expressed in Escherichia coli. Nature Biotechnol., 25, 563-565 (2007).
Meijer et al., Isolation of human antibody repertoires with preservation of the natural heavy and light chain pairing. J. Mol. Biol., 358, 764-772 (2006).
Morozov, Dao Thi, & Denner, The transmembrane protein of the human endogenous retrovirus-K (HERV-K) modulates cytokine release and gene expression. PLOS One. 8, e70399 (2013).
Natsume, Niwa, & Satoh, Improving effector functions of antibodies for cancer treatment: Enhancing ADCC and CDC. Drug Des Devel Ther. 2009; 3:7-16.
Novak et al., Safety and immunogenicity of the tau vaccine AADvac1 in patients with Alzheimer's disease: A randomised, double-blind, placebo-controlled, phase 1 trial. Lancet Neurol., 16, 123-134 (2017).
Ono, Kawakami, & Ushikubo, Stimulation of expression of the human endogenous retrovirus genome by female steroid hormones in human breast cancer cell line T47D. J Virol. 1987; 61:2059-2062.
Ono, Yasunaga, Miyata, & Ushikubo, Nucleotide sequence of human endogenous retrovirus genome related to the mouse mammary tumor virus genome. J. Virol. 60, 589-598 (1986). The most biologically active HERVs are members of the HERV-K family. HERV-K has a complete sequence capable of expressing all the elements needed for a replication-competent retrovirus.
Oricchio et al., Distinct roles for LINE-1 and HERV-K retroelements in cell proliferation, differentiation and tumor progression. Oncogene, 26, 4226-4233 (2007).
Robinson-Mccarthy et al., Reconstruction of the cell entry pathway of an extinct virus. PLOS Pathog., 14, e1007123 (2018).
Rycaj et al., Cytotoxicity of human endogenous retrovirus K-specific T cells toward autologous ovarian cancer cells. Clin Cancer Res. 2015; 21:471-483. The inventors observed the expression of HERVs, especially HERV-K sequences, in breast, lung, prostate, ovarian, colon, pancreatic, and other solid tumors.
Scheid et al., Broad diversity of neutralizing antibodies isolated from memory B cells in HIV-infected individuals. Nature, 458, 636-640 (2009).
Seifarth et al., Retrovirus-like particles released from the human breast cancer cell line T47-D display type B- and C-related endogenous retroviral sequences. J Virol. 1995; 69:6408-6416.
Seitz et al., Reconstitution of paired T cell receptor alpha- and beta-chains from microdissected single cells of human inflammatory tissues. Proc. Natl. Acad. Sci. USA, 103, 12057-12062 (2006).
Serafino et al., The activation of human endogenous retrovirus K (HERV-K) is implicated in melanoma cell malignant transformation. Exp. Cell Res., 315, 849-862 (2009).
Smith et al., Rapid generation of fully human monoclonal antibodies specific to a vaccinating antigen. Nature Protoc., 4, 372-384 (2009).
Song & Liu, A TLR9 agonist enhances the anti-tumor immunity of peptide and lipopeptide vaccines via different mechanisms. Scientific Reports, 5, 12578 (2015).
Spector & Blackwell, Understanding the mechanisms behind trastuzumab therapy for human epidermal growth factor receptor 2-positive breast cancer. J. Clinical Oncol. 2009; 27:5838-5847.
Wallace et al., Elevated HERV-K mRNA expression in PBMC is associated with a prostate cancer diagnosis particularly in older men and smokers. Carcinogenesis., 35, 2074-2083 (2014).
Wang-Johanning et al., Detecting the expression of human endogenous retrovirus E envelope transcripts in human prostate adenocarcinoma. Cancer, 98, 187-197 (2003). The inventors observed the expression of HERVs, especially HERV-K sequences, in breast, lung, prostate, ovarian, colon, pancreatic, and other solid tumors.
Wang-Johanning et al., Expression of human endogenous retrovirus k envelope transcripts in human breast cancer. Clin. Cancer Res. 7, 1553-1560 (2001).
Wang-Johanning et al., Expression of multiple human endogenous retrovirus surface envelope proteins in ovarian cancer. Int J Cancer. 2007; 120:81-90.
Wang-Johanning et al., Human endogenous retrovirus K triggers an antigen-specific immune response in breast cancer patients. Cancer Res. 2008; 68:5869-5877. The inventors observed the expression of HERVs, especially HERV-K sequences, in breast, lung, prostate, ovarian, colon, pancreatic, and other solid tumors.
Wang-Johanning et al., Human endogenous retrovirus type K antibodies and mRNA as serum biomarkers of early-stage breast cancer. Int. J. Cancer. 2014; 134:587-595.
Wang-Johanning et al., Immunotherapeutic potential of anti-human endogenous retrovirus-K envelope protein antibodies in targeting breast tumors. J. Natl. Cancer Inst., 104, 189-210 (2012). The inventors showed that the HERV-K Env protein is commonly expressed on the surface of breast cancer cells. The inventors observed the expression of HERVs, especially HERV-K sequences, in breast, lung, prostate, ovarian, colon, pancreatic, and other solid tumors.
Wang-Johanning et al., Quantitation of HERV-K env gene expression and splicing in human breast cancer. Oncogene, 22, 1528-1535 (2003). The inventors observed the expression of HERVs, especially HERV-K sequences, in breast, lung, prostate, ovarian, colon, pancreatic, and other solid tumors.
Wang-Johanning et al., Tumor microenvironment predicts aggressive breast cancer: Combination of HERV-K, immune checkpoint and activation status of CD8+ T cells. Cancer Res., 77, Abstract nr LB-221 (2017).
Zahavi, Monoclonal Antibodies in Cancer Therapy. MDPI Web. (Jul. 20, 2020).
Zhang et al., An EpCAM/CD3 bispecific antibody efficiently eliminates hepatocellular carcinoma cells with limited galectin-1 expression. Cancer Immunol. Immunother., 63, 121-132 (2014).
Zhao et al., Expression of Human Endogenous Retrovirus Type K Envelope Protein is a Novel Candidate Prognostic Marker for Human Breast Cancer. Genes Cancer, 2:914-922 (2011). The inventors observed the expression of HERVs, especially HERV-K sequences, in breast, lung, prostate, ovarian, colon, pancreatic, and other solid tumors.
Zhou et al., Activation of HERV-K Env protein is essential for tumorigenesis and metastasis of breast cancer cells. Oncotarget, 7, 84093-84117 (2016).
Zhou et al., Chimeric antigen receptor T cells targeting HERV-K inhibit breast cancer and its metastasis through downregulation of Ras. Oncoimmunology, 4, e1047582 (2015). The inventors observed the expression of HERVs, especially HERV-K sequences, in breast, lung, prostate, ovarian, colon, pancreatic, and other solid tumors.
Zhou, Zou, Zhang, & Marks, Internalizing cancer antibodies from phage libraries selected on tumor cells and yeast-displayed tumor antigens. J. Mol. Biol., 2010; 404:88-99.

SEQUENCE LISTING
Sequence of the Mouse ScFV gene
SEQ ID NO: 1

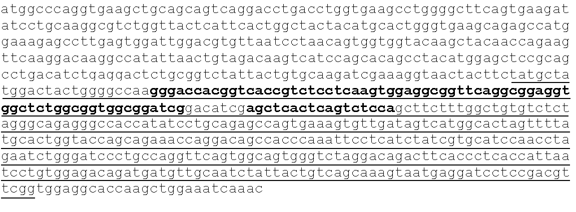

Sequence of the Mouse ScFV gene: VH
SEQ ID NO: 2
caggtgaagctgcagcagtcaggacctgacctggtgaagcctggggcttcagtgaagatatcctg
caaggcgtctggttactcattcactggctactacatgcactgggtgaagcagagccatggaaaga
gccttgagtggattggacgtgttaatcctaacagtggtggtacaagctacaaccagaagttcaag
gacaaggccatattaactgtagacaagtcatccagcacagcctacatggagctccgcagcctgac
atctgaggactctgcggtctattactgtgcaagatcgaaaggtaactacttctatgctatggact
actggggccaagggaccacggtcaccgtctcctcaa
Sequence of the Mouse ScFV gene: >FWJ_VH
SEQ ID NO: 3
VH CDRs SCKASGYSFTGYYMHWV---IGRVNPNSGGTSYNQKFKD---CARSKGNYFYAMDY
QVKLQQSGPDLVKPGASVKISCKASGYSFTGYYMHWVKQSHGKSLEWIGRVNPNSGGTSYNQKFK
DKAILTVDKSSSTAYMELRSLTSEDSAVYYCARSKGNYFYAMDYWGQGTTVTVSS
Sequence of the Mouse ScFV gene: VL
SEQ ID NO: 4
gacatcgagctcactcagtctccagcttctttggctgtgtctctagggcagagggccaccatatc
ctgcagagccagtgaaagtgttgatagtcatggcactagttttatgcactggtaccagcagaaac
caggacagccacccaaattcctcatctatcgtgcatccaacctagaatctgggatccctgccagg
ttcagtggcagtgggtctaggacagacttcaccctcaccattaatcctgtggagacagatgatgt
tgcaatctattactgtcagcaaagtaatgaggatcctccgacgttcggtggaggcaccaagctgg
aaatcaaac
Sequence of the Mouse ScFV gene: >FWJ_VL
SEQ ID NO: 5
DIELTQSPASLAVSLGQRATISCRASESVDSHGTSFMHWYQQKPGQPPKFLIYRASNLESGIPAR
FSGSGSRTDFTLTINPVETDDVAIYYCQQSNEDPPTFGGGTKLEIK
Sequence of the Humanized ScFV gene: >HUM1-FWJVH
SEQ ID NO: 6
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSS
Sequence of the Humanized ScFV gene: >Hum2 FWJVH VH
SEQ ID NO: 7
EVQLVESGGGLVQPGGSLKVSCKASGYSFTGYYMHWVRQASGKGLEWIGRVNPNSGGTSYNQKFK
DRFTISRDKSISTLYLQMSSLRSEDTAVYYCARSKGNYFYAMDYWGQGTLVTVSS
Sequence of the Mouse ScFV gene: >FWJ_VH
SEQ ID NO: 8
QVKLQQSGPDLVKPGASVKISCKASGYSFTGYYMHWVKQSHGKSLEWIGRVNPNSGGTSYNQKFK
DKAILTVDKSSSTAYMELRSLTSEDSAVYYCARSKGNYFYAMDYWGQGTTVTVSS
Sequence of the Humanized ScFV gene: >HUM1FWJVL
SEQ ID NO: 9
DIQMTQSPSSLSASVGDRVTITCRASESVDSHGTSFMHWYQQKPGKAPKFLIYRASNLESGIPSR
FSGSGSGTDFTLTISSVQPEDFAVYYCQQSNEDPPTFGGGTKVEIK
Sequence of the Humanized ScFV gene: >HUM2FWJVL
SEQ ID NO: 10
DIQMTQSPSSLSASVGDRVTISCRASESVDSHGTSFMHWYQQKPGKSPKFLIYRASNLESGIPSR
FSGSGSGTDFTLTISSLQPEDFAIYYCQQSNEDPPTFGGGTKVEIK
Sequence of the Mouse ScFV gene: >FWJ_VL
SEQ ID NO: 11
DIELTQSPASLAVSLGQRATISCRASESVDSHGTSFMHWYQQKPGQPPKFLIYRASNLESGIPAR
FSGSGSRTDFTLTINPVETDDVAIYYCQQSNEDPPTFGGGTKLEIK
Final humanized version of scFv gene scFv-1
SEQ ID NO: 12
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSSGGGGSGGGGS
GGGGSDIQMTQSPSSLSASVGDRVTITCRASESVDSHGTSFMHWYQQKPGKAPKFLIYRASNLES
GIPSRFSGSGSSGTDFTLTISSVQPEDFAVYYCQQSNEDPPTFGGGTKVEIK
Final humanized version of scFv gene scFv-2
SEQ ID NO: 13
EVQLVESGGGLVQPGGSLKVSCKASGYSFTGYYMHWVRQASGKGLEWIGRVNPNSGGTSYNQKFK
DRFTISRDKSISTLYLQMSSLRSEDTAVYYCARSKGNYFYAMDYWGQGTLVTVSSGGGGSGGGGS
GGGGSDIQMTQSPSSLSASVGDRVTISCRASESVDSHGTSFMHWYQQKPGKSPKFLIYRASNLES
GIPSRFSGSSGSGTDFTLTISSLQPEDFAIYYCQQSNEDPPTFGGGTKVEIK
Standard 20 amino acid linker
SEQ ID NO: 14
GGGGSGGGGSGGGGSGGGAR
Final humanized version of scFv gene: Humanized scFv-1 FWJ_humscFv-1
SEQ ID NO: 15
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSSGGGGSGGGGS
GGGGSDIQMTQSPSSLSASVGDRVTITCRASESVDSHGTSFMHWYQQKPGKAPKFLIYRASNLES
GIPSRFSGSGSGTDFTLTISSVQPEDFAVYYCQQSNEDPPTFGGGTKVEIK
Final humanized version of scFv gene: >Humanized scFv-
1_polynucleotide
SEQ ID NO: 16

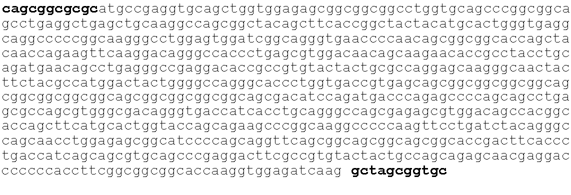

Final humanized version of scFv gene
SEQ ID NO: 17
gaggtgcagctggtggagagcggcggcggcctggtgcagcccggcggcagcctgaggctgagctg
caaggccagcggctacagcttcaccggctactacatgcactgggtgaggcaggcccccggcaagg
gcctggagtggatcggcagggtgaaccccaacagcggcggcaccagctacaaccagaagttcaag
gacagggccaccctgagcgtggacaacagcaagaacaccgcctacctgcagatgaacagcctgag
ggccgaggacaccgccgtgtactactgcgccaggagcaagggcaactacttctacgccatggact
actggggccagggcaccctggtgaccgtgagcagcggcggcggcggcagcggcggcggcggcagc
ggcggcggcggcagcgacatccagatgacccagagccccagcagcctgagcgccagcgtgggcga
cagggtgaccatcacctgcagggccagcgagagcgtggacagccacggcaccagcttcatgcact
ggtaccagcagaagcccggcaaggcccccaagttcctgatctacagggccagcaacctggagagc
ggcatccccagcaggttcagcggcagcggcagcggcaccgacttcaccctgaccatcagcagcgt
gcagcccgaggacttcgccgtgtactactgccagcagagcaacgaggacccccccaccttcggcg
gcggccaaggtggagatcaag
Final humanized version of scFv gene: Humanized FWJ_humscFv-2
SEQ ID NO: 18
EVQLVESGGGLVQPGGSLKVSCKASGYSFTGYYMHWVRQASGKGLEWIGRVNPNSGGTSYNQKFK
DRFTISRDKSISTLYLQMSSLRSEDTAVYYCARSKGNYFYAMDYWGQGTLVTVSSGGGGSGGGGS
GGGGSDIQMTQSPSSLSASVGDRVTISCRASESVDSHGTSFMHWYQQKPGKSPKFLIYRASNLES
GIPSRFSGSGSGTDFTLTISSLQPEDFAIYYCQQSNEDPPTFGGGTKVEIK
Final humanized version of scFv gene: >>Humanized scFv-2_polypeptide
SEQ ID NO: 19

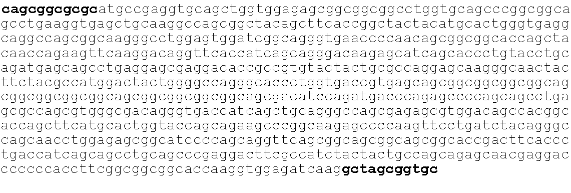

Final humanized version of scFv gene: Protein
SEQ ID NO: 20
GAGGTGCAGCTGGTGGAGAGCGGCGGCGGCCTGGTGCAGCCCGGCGGCAGCCTGAAGGTGAGCTG
CAAGGCCAGCGGCTACAGCTTCACCGGCTACTACATGCACTGGGTGAGGCAGGCCAGCGGCAAGG
GCCTGGAGTGGATCGGCAGGGTGAACCCCAACAGCGGCGGCACCAGCTACAACCAGAAGTTCAAG
GACAGGTTCACCATCAGCAGGGACAAGAGCATCAGCACCCTGTACCTGCAGATGAGCAGCCTGAG
GAGCGAGGACACCGCCGTGTACTACTGCGCCAGGAGCAAGGGCAACTACTTCTACGCCATGGACT
ACTGGGGCCAGGGCACCCTGGTGACCGTGAGCAGCGGCGGCGGCGGCAGCGGCGGCGGCGGCAGC
GGCGGCGGCGGCAGCGACATCCAGATGACCCAGAGCCCCAGCAGCCTGAGCGCCAGCGTGGGCGA
CAGGGTGACCATCAGCTGCAGGGCCAGCGAGAGCGTGGACAGCCACGGCACCAGCTTCATGCACT
GGTACCAGCAGAAGCCCGGCAAGAGCCCCAAGTTCCTGATCTACAGGGCCAGCAACCTGGAGAGC
GGCATCCCCAGCAGGTTCAGCGGCAGCGGCAGCGGCACCGACTTCACCCTGACCATCAGCAGCCT
GCAGCCCGAGGACTTCGCCATCTACTACTGCCAGCAGAGCAACGAGGACCCCCCCACCTTCGGCG
GCGGCACCAAGGTGGAGATCAAG
OKT8 Heavy chains: (Anti-CD8 mAb sequence): >H1
SEQ ID NO: 21
tttgaggtccagctgcagcagtctggggcagagcttgtgaagccaggggcctcagtcaagttgtc
ctgcacagcttctggcttcaacattaaagacacctatatacacttcgtgaggcagaggcctgaac
agggcctggagtggattggaaggattgatcctgcgaatgataatactttatatgcctcaaagttc
cagggcaaggccactataacagcagacacatcatccaacacagcctacatgcacctctgcagcct
gacatctggggacactgccgtctattactgtggtagaggttatggttactacgtatttgaccact
tgggccaaggcnntnnnnntnncannntnnnnnnn
OKT8 Heavy chains: (Anti-CD8 mAb sequence): >H1
SEQ ID NO: 22
FEVQLQQSGAELVKPGASVKLSCTASGFNIKDTYIHFVRQRPEQGLEWIGRIDPANDNTLYASKF
QGKATITADTSSNTAYMHLCSLTSGDTAVYYCGRGYGYYVFDHLGQGXXXXXXXX
OKT8 Heavy chains: (Anti-CD8 mAb sequence): >H3
SEQ ID NO: 23
tntcaggtgcagctgaagcagtctggggcagagcttgtgaagccaggggcctcagtcaagttgtc
ctgcacagcttctggcttcaacattaaagacacctatatacacttcgtgaggcagaggcctgaac
agggcctggagtggattggaaggattgatcctgcgaatgataatactttatatgcctcaaagttc
cagggcaaggccactataacagcagacacatcatccaacacagcctacatgcacctctgcagcct
gacatctggggacactgccgtctattactgtggtagaggttatggttactacgtatttgaccact
ggggccaaggcaccactctcacantnncnnnn
OKT8 Heavy chains: (Anti-CD8 mAb sequence): >3
SEQ ID NO: 24
XQVQLKQSGAELVKPGASVKLSCTASGFNIKDTYIHFVRQRPEQGLEWIGRIDPANDNTLYADKF
QGKATITADTSSNTAYMHLCSLTSGDTAVYYCGRGYGYYVFDHWGQGTTLTXXX
OKT8 Heavy chains: (Anti-CD8 mAb sequence): >H6
SEQ ID NO: 25
tttgaggtccagctgcagcagtctggggcagagcttgtgaagccaggggcctcagtcaagttgtc
ctgcacagcttctggcttcaacattaaagacacctatatacacttcgtgaggcagaggcctgaac
agggcctggagtggattggaaggattgatcctgcgaatgataatactttatatgcctcaaagttc
cagggcaaggccactataacagcagacacatcatccaacacagcctacatgcacctctgcagcct
gacatctggggacactgccgtctattactgtggtagaggttatggttactacgtatttgaccact
ggggccaaggcaccactctcacantnncnnna
OKT8 Heavy chains: (Anti-CD8 mAb sequence): >H6
SEQ ID NO: 26
FEVQLQQSGAELVKPGASVKLSCTASGFNIKDTYIHFVRQRPEQGLEWIGRIDPANDNTLYASKF
QGKATITADTSSNTAYMHLCSLTSGDTAVYYCGRGYGYYVFDHWGQGTTLTXXX
OKT8 Heavy chains: (Anti-CD8 mAb sequence): >H7
SEQ ID NO: 27
tcaggtccaactgcagcagcctggggcagagcttgtgaagccaggggcctcagtcaagttgtcct
gcacagcttctggcttcaacattaaagacacctatatacacttcgtgaggcagaggcctgaacag
ggcctggagtggattggaaggattgatcctgcgaatgataatactttatatgcctcaaagttcca
gggcaaggccactataacagcagacacatcatccaacacagcctacatgcacctctgcagcctga
catctggggacactgccgtctattactgtggtagaggttatggttactacgtatttgaccactgg
ggccaaggcaccactctcacatntnnn
OKT8 Heavy chains: (Anti-CD8 mAb sequence): >H7
SEQ ID NO: 28
QVQLQQPGAELVKPGASVKLSCTASGFNIKDTYIHFVRQRPEQGLEWIGRIDPANDNTLYASKFQ
GKATITADTSSNTAYMHLCSLTSGDTAVYYCRGYGYYVFDHWGQGTTLTXX
CD8 BiTE
SEQ ID NO: 29
accggtatggatatcgagctgacccagagccctagcagcctggccgtgtcactgggccagagagc
caccatcagctgcagagcctccgagagcgtggatagccacggcaccagcctgatgcactggtatc
agcagaagcccggccagccccccaagttcctgatctaccgggccagcaacctggaaagcggcatc
cccgccagattttccggcagcggcagcagaaccgacttcaccctgaccatcaaccccgtggagac
agacgacgtggccatctactactgccagcagagcaacgaggaccctcccacctttggcggaggca
ccaagctggaactgaaggagggcggaggaagcggagggggaggatctggcggaggcggcagcgcc
caggtgaagctgcagcagagcggccctgatctggtgaagcctggcgccagcgtgaagatcagctg
caaggccagcggctacagcttcaccggctactacatgcactgggtgaaacagagccacggcaaga
gcctggaatggatcggcagagtgaaccccaatagcggcggcaccagctacaaccagaagttcaag
gacaaggccatcctgaccgtggacaagagcagcagcaccgcctacatggaactgcggagcctgac
cagcgaggacagcgccgtgtactactgcgcccggtccaagggcaactacttctacgccatggact
actggggccagggcaccaccgtgaccgtgtctagcagcggcggcggcggaagcgaagtgcagctg
cagcagtccggagcagaactggtgaaacccggagccagtgtgaagctgagctgtacagcctccgg
ctttaatatcaaggacacctacatccacttcgtgcgccaacggccagaacagggtctggagtgga
ttggcaggatcgatccagcaaatgataacaccctgtacgcaagcaaatttcagggcaaagccacg
ataaccgccgatacatctagtaatacggcttacatgcacctctgctccctgacttccggggacac
cgccgtgtattattgcgggcgcggatacggttactacgtgttcgatcattggggtcagggcacca
ccctcacagtctccagtgcaggaggcggaggtagtggaggcggaggctctggcggtggcgggtca
gacatagtgctgacacagtctcccgccagtctggcagtgagccttggccagagggctacaatttc
ctatagggcgtctaaatccgttagcacttcaggttactcttatatgcactggaaccagcagaagc
ctggtcagccccccaggcttcttatttacctggtcagcaatctcgagtctggcgtgcccgctaga
ttttccggcagcgggagtgggactgacttcactctgaacatccacccagtggaggaagaggatgc
cgcaacctactattgtcagcatatccgggaattgaccagatcagagggcggaccttcctggaagg
agcagaagctgattagcgaggaagatctggattataaggacgacgatgacaaatgagaattc
CD8 BiTE
SEQ ID NO: 30
TGMDIELTQSPSSLAVSLGQRATISCRASESVDSHGTSLMHWYQQKPGQPPKFLIYRASNLESGI
PARFSGSGSRTDFTLTINPVETDDVAIYYCQQSNEDPPTFGGGTKLELKEGGGSGGGGSGGGGSA
QVKLQQSGPDLVKPGASVKISCKASGYSFTGYYMHWVKQSHGKSLEWIGRVNPNSGGTSYNQKFK
DKAILTVDKSSSTAYMELRSLTSEDSAVYYCARSKGNYFYAMDYWGQGTTVTVSSSGGGGSEVQL
QQSGAELVKPGASVKLSCTASGFNIKDTYIHFVRQRPEQGLEWIGRIDPANDNTLYASKFQGKAT
ITADTSSNTAYMHLCSLTSGDTAVYYCGRGYGYYVFDHWGQGTTLTVSSAGGGGSGGGGSGGGGS
DIVLTQSPASLAVSLGQRATISYRASKSVSTSGYSYMHWNQQKPGQPPRLLIYLVSNLESGVPAR
FSGSGSGTDFTLNIHPVEEEDAATYYCQHIRELTRSEGGPSWKEQKLISEEDLDYKDDDDKEF
CD3 BiTE
SEQ ID NO: 31
accggtatggatatcgagctgacccagagccctagcagcctggccgtgtcactgggccagagagc
caccatcagctgcagagcctccgagagcgtggatagccacggcaccagcctgatgcactggtatc
agcagaagcccggccagccccccaagttcctgatctaccgggccagcaacctggaaagcggcatc
cccgccagattttccggcagcggcagcagaaccgacttcaccctgaccatcaaccccgtggagac
agacgacgtggccatctactactgccagcagagcaacgaggaccctcccacctttggcggaggca
ccaagctggaactgaaggagggcggaggaagcggagggggaggatctggcggaggcggcagcgcc
caggtgaagctgcagcagagcggccctgatctggtgaagcctggcgccagcgtgaagatcagctg
caaggccagcggctacagcttcaccggctactacatgcactgggtgaaacagagccacggcaaga
gcctggaatggatcggcagagtgaaccccaatagcggcggcaccagctacaaccagaagttcaag
gacaaggccatcctgaccgtggacaagagcagcagcaccgcctacatggaactgcggagcctgac
cagcgaggacagcgccgtgtactactgcgcccggtccaagggcaactacttctacgccatggact
actggggccagggcaccaccgtgaccgtgtctagcagcggcggcggcggaagcgaggttcagctg
gtggagtctggcggtggcctggtgcagccagggggctcactccgtttgtcctgtgcagcttctgg
ctactcctttaccggctacactatgaactgggtgcgtcaggccccaggtaagggcctggaatggg
ttgcactgattaatccttataaaggtgtttccacctataaccagaaattcaaggatcgtttcacg
atatccgtagataaatccaaaaacacagcctacctgcaaatgaacagcctgcgtgctgaggacac
tgccgtctattattgtgctagaagcggatactacggcgatagcgactggtattttgacgtctggg
gtcaaggaaccctggtcaccgtctcctcgggtggaggcggttcaggcggaggtggctctggcggt
ggcggatcggatatccagatgacccagtccccgagctccctgtccgcctctgtgggcgatagggt
caccatcacctgtcgtgccagtcaggacatccgtaattatctcaactggtatcaacagaaaccag
gaaaagctccgaaactactgatttactatacctcccgcctggagtctggagtcccttctcgcttc
tctggttctggttctgggacggattacactctgaccatcagcagtctgcaaccggaggacttcgc
aacttattactgtcagcaaggtaatactctgccgtggacgttcggacagggcaccaaggtggaga
tcaaagagcagaagctgattagcgaggaagatctggattataaggacgacgatgacaaatgagaa
ttc
CD3 BiTE
SEQ ID NO: 32
TGMDIELTQSPSSLAVSLGQRATISCRASESVDSHGTSLMHWYQQKPGQPPKFLIYRASNLESGI
PARFSGSGSRTDFTLTINPVETDDVAIYYCQQSNEDPPTFGGGTKLELKEGGGSGGGGSGGGGSA
QVKLQQSGPDLVKPGASVKISCKASGYSFTGYYMHWVKQSHGKSLEWIGRVNPNSGGTSYNQKFK
DKAILTVDKSSSTAYMELRSLTSEDSAVYYCARSKGNYFYAMDYWGQGTTVTVSSSGGGGSEVQL
VESGGGLVQPGGSLRLSCAASGYSFTGYTMNWVRQAPGKGLEWVALINPYKGVSTYNQKFKDRFT
ISVDKSKNTAYLWMNSLRAEDTAVYYCARSGYYGDSDWYFDVWGQGTLVTVSSGGGGSGGGGSGG
GGSDIQMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKEQKLISEEDLDYKDDDDKEF
FLAG-tag
SEQ ID NO: 33
DYKDDDDK
Myc-tag
SEQ ID NO: 34
EQKLISEEDL
Heavy chain sequence
Color scheme: Signal peptide-VH-CH (Human IgG1)
SEQ ID NO: 35
GCGGCCGCAAACTACAAGACAGACTTGCAAAAGAAGGCATGCACAGCTCAGCACTGCTCTGTTGC
CTGGTCCTCCTGACTGGGGTGAGGGCCGAGGTGCAGCTGGTGGAGAGCGGGGGGGGACTGGTGCA
GCCAGGAGGAAGCCTGAGACTGAGCTGTAAGGCTTCAGGATACAGCTTCACAGGGTACTATATGC
ACTGGGTGAGACAGGCCCCCGGAAAAGGACTGGAGTGGATCGGCAGAGTGAACCCCAACAGCGGA
GGCACCAGCTACAACCAGAAATTCAAGGACCGGGCCACCCTGAGCGTGGACAACAGCAAAAACAC
AGCCTACCTGCAGATGAACAGCCTGCGGGCCGAGGACACCGCCGTGTACTATTGTGCCAGAAGCA
AGGGCAACTACTTCTACGCCATGGACTATTGGGGCCAGGGAACACTGGTGACCGTGAGCAGCGCT
AGCACCAAGGGCCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAAACTCACACATGCC
CACCGTGCCCAGCACCTGAACTCCTGGGGGGACCGTCAGTCTTCCTCTTCCCCCCAAAACCCAAG
GACACCCTCATGATCTCCCGGACCCCCGAGGTCACATGCGTGGTGGTGGACGTGAGCCACGAAGA
CCCTGAGGTCAAGTTCAACTGGTACGTGGACGGCGTGGAGGTGCATAATGCCAAGACAAAGCCGC
GGGAGGAGCAGTACAACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCATCCCGGGAGG
AGATGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTC
CGACGGCTCCTTCTTCCTCTACAGCAAGCTCACCGTGGACAAGAGCAGGTGGCAGCAGGGGAACG
TCTTCTCATGCTCCGTGATGCATGAGGCTCTGCACAACCACTACACGCAGAAGAGCCTCTCCCTG
TCTCCGGGTAAATGATTCTAGA
Amino acid sequence:
SEQ ID NO: 36
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSSASTKGPSVFP
LAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSPGK
Light chain sequence
Color scheme: Signal peptide-VL-CL (Human Kappa)
SEQ ID NO: 37
GCGGCCGCAAACTACAAGACAGACTTGCAAAAGAAGGCATGCACAGCTCAGCACTGCTCTGTTGC
CTGGTCCTCCTGACTGGGGTGAGGGCCGACATTCAGATGACCCAGAGCCCCAGCAGCCTGAGCGC
CAGCGTGGGAGACAGAGTGACCATCACCTGCAGAGCCAGCGAGAGCGTGGACAGCCATGGCACCT
CCTTTATGCACTGGTACCAGCAGAAGCCCGGCAAGGCCCCCAAATTCCTGATTTACAGAGCCAGC
AACCTGGAGAGCGGCATCCCCTCAAGATTCTCAGGCAGCGGAAGCGGAACCGACTTCACACTGAC
TATCAGCAGCGTGCAGCCCGAAGATTTTGCTGTGTACTACTGCCAGCAGAGCAACGAAGATCCTC
CTACCTTCGGGGGCGGCACTAAGGTGGAGATCAAGCGTACGGTGGCTGCACCATCTGTCTTCATC
TTCCCGCCATCTGATGAGCAGTTGAAATCTGGAACTGCCTCTGTTGTGTGCCTGCTGAATAACTT
CTATCCCAGAGAGGCCAAAGTACAGTGGAAGGTGGATAACGCCCTCCAATCGGGTAACTCCCAGG
AGAGTGTCACAGAGCAGGACAGCAAGGACAGCACCTACAGCCTCAGCAGCACCCTGACGCTGAGC
AAAGCAGACTACGAGAAACACAAAGTCTACGCCTGCGAAGTCACCCATCAGGGCCTGAGTTCGCC
CGTCACAAAGAGCTTCAACAGGGGAGAGTGTTGATTCTAGA
Amino acid sequence
SEQ ID NO: 38
DIQMTQSPSSLSASVGDRVTITCRASESVDSHGTSFMHWYQQKPGKAPKFLIYRASNLESGIPSR
FSGSGSGTDFTLTISSVQPEDFAVYYCQQSNEDPPTFGGGTKVEIKRTVAAPSVFIFPPSDEQLK
SGTASVVCLLNNFYPREAKVQWKVDNALQSGNSQESVTEQDSKDSTYSLSSTLTLSKADYEKHKV
YACEVTHQGLSSPVTKSFNRGEC
scFv against MAPs of HERV-K (sequence for anti-HERV-K mAb)
SEQ ID NO: 39
atggcccaggtgcaactgcagcagtcaggggctgagcttgtgaagcctggggcttcagtgaagat
gtcctgcaaggcttctggctacaccttcaccagctactggataacctgggtgaagcagaggcctg
gacaaggccttgagtggattggagatatttatcctggtagtggtagtactaactacaatgagaag
ttcaagagcaaggccacactgactgtagacacatcctccagcacagcctacatgcagctcagcag
cctgacatctgaggactctgcggtctattactgtgcaagatggcgggacgggtactatgctatgg
actactggggccaagggaccacggtcaccgtctcctcaagtgaaggcggttcaggcggaggttgc
tctggcggtggcggatcggacatcgagctcactcagtctccaaccaccatggctgcatctcccgg
ggagaagatcactatcacctgcagtgccagctcaagtataagttccaattacttgcattggtatc
agcagaagccaggattctcccctaaactcttgatttataggacatccaatctggcttctggagtc
ccagctcgcttcagtggcagtgggtctgggacctcttactctctcacaattggcaccatggaagc
tgaagatgttgccacttactactgccagcagggtagtagtataccattcacgttcggctcgggga
cgaagttggagctgaaacgggcgggccgcaggtgcgccggtgccgtatccg
scFv against MAPs of HERV-K (sequence for anti-HERV-K mAb)
SEQ ID NO: 40
MAQVQLQQSGAELVKPGASVKMSCKASGYTFTSYWITWVKQRPGQGLEWIGDIYPGSGSTNYNEK
FKSKATLTVDTSSSTAYMQLSSLTSEDSAVYYCARWRDGYYAMDYWGQGTTVTVSSSEGGSGGGC
SGGGGSDIELTQSPTTMAASPGEKITITCSASSSISSNYLHWYQQKPGFSPKLLIYRTSNLASGV
PARFSGSGSGTSYSLTIGTMEAEDVATYYCQQGSSIPFTFGSGTKLELKRAGRRCAGAVS
Humanized FWJ humscFv-2
SEQ ID NO: 41
EVQLVESGGGLVQPGGSLKVSCKASGYSFTGYYMHWVRQASGKGLEWIGRVNPNSGGTSYNQKFK
DRFTISRDKSISTLYLQMSSLRSEDTAVYYCARSKGNYFYAMDYWGQGTLVTVSSGGGGSGGGGS
GGGGSDIQMTQSPSSLSASVGDRVTISCRASESVDSHGTSFMHWYQQKPGKSPKFLIYRASNLES
GIPSRFSGSGSGTDFTLTISSLQPEDFAIYYCQQSNEDPPTFGGGTKVEIK
>>Codon for Humanized scFv-2 nucleotide
SEQ ID NO: 42

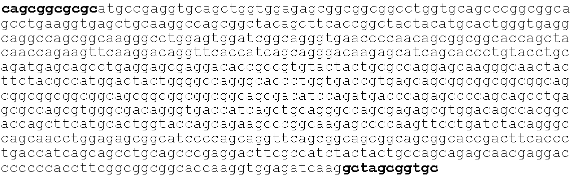

Env sequence from breast cancer patient viral peptide.
SEQ ID NO: 43
gtaacaccagtcacatggatggataatcctatagaagtatatgttaatgatagtgtatgg
gtacctggccccacagatgatcgctgccctgccaaacctgaggaagaagggatgatgata
Env sequence from breast cancer patient viral peptide.
SEQ ID NO: 44
aatatttccattgggtatcgttatcctcctatttgcctagggagagcaccaggatgtttaatgcc
tgcagtccaaaattggttggtagaagtacctactgtcagtcccatcagtagattcacttatcaca
tggtaagcgggatgtcactcaggccacgggtaaattatttacaagacttttcttatcaaagatca
ttaaaatttagacctaaagggaaaccttgccccaaggaaattcccaaagaatcaaaaaatacaga
agttttagtttgggaagaatgtgtggccaatagtgcggtgatattacaaaacaatgaattcggaa
ctattatagattgggcacctcgaggtcaattctaccacaattgctcaggacaaactcagtcgtgt
ccaagtgcacaagtgagtccagctgttgatagcgacttaacagaaagtttagacaaacataagca
taaaaaattgcagtctttctacccttgggaatggggagaaaaaggaatctctaccccaagaccaa
aaataataagtcctgtttctggtcctgaacatccagaattatggaggcttactgtggcctcacac
cacattagaatttggtctggaaatcaaactttagaaacaagagatcgtaagccattttatactgt
cgacctaaattccagtctaacagttcctttacaaagttgcgtaaagcccccttatatgctagttg
taggaaatatagttattaaaccagactcccaaactataacctgtgaaaattgtagattgcttact
tgcattgattcaacttttaattggcaacaccgtattctgctggtgagagcaagagagggcgtgtg
gatccctgtgtccatggaccgaccgtgggaggcctcaccatccgtccatattttgactgaagtat
taaaaggtgttttaaatagatccaaaagattcatttttactttaattgcagtgattatgggatta
attgcagtcacagctacggctgctgtagcaggagttgcattgcactcttctgttcagtcagtaaa
ctttgttaatgattggcaaaaaaattctacaagattgtggaattcacaatctagtattgatcaaa
aattggcaaatcaaattaatgatcttagacaaactgtcatttggatgggagacagactcatgagc
ttagaacatcgtttccagttacaatgtgactggaatacgtcagatttttgtattacaccccaaat
ttataatgagtctgagcatcactgggacatggttagacgccatctacagggaagagaagataatc
tcactttagacatttccaaattaaaagaacaaattttcgaagcatcaaaagcccatttaaatttg
gtgccaggaactgaggcaattgcaggagttgctgatggcctcgcaaatcttaaccctgtcacttg
ggttaagaccattggaagtactacgattataaatctcatattaatccttgtgtgcctgttttgtc
tgttgttagtctgcaggtgtacccaacagctccgaagagacagcgaccatcgagaacgggccatg
atgacgatggcggttttgtcgaaaagaaaagggggaaatgtggggaaaagcaagagagatcaaat
tgttactgtgtctgtgtag
Env sequence from breast cancer patient viral peptide.
SEQ ID NO: 45
VTPVTWMDNPIEVYVNDSVWVPGPTDDRCPAKPEEEGMMINISIGYRYPPICLGRAPGCLMPAVQ
NWLVEVPTVSPISRFTYHMVSGMSLRPRVNYLQDFSYQRSLKFRPKGKPCPKEIPKESKNTEVLV
WEECVANSAVILQNNEFGTIIDWAPRGQFYHNCSGQTQSCPSAQVSPAVDSDLTESLDKHKHKKL
QAFYPWEWGEKGISTPRPKIISPVSGPEHPELWRLTVASHHIRIWSGNQTLETRDRKPFYTVDLN
SSLTVPLQSCVKPPYMLVVGNIVIKPDSQTITCENCRLLTCIDSTFNWQHRILLVRAREGVWIPV
SMDRPWEASPSVHILTEVLKGVLNRSKRFIFTLIAVIMGLIAVTATAAVAGVALHSSVQSVNFVN
DWQKNSTRLWNSQSSIDQKLANQINDLRQTVIWMGDRLMSLEHRFQLQCDWNTSDFCITPQIYNE
SEHHWDMVRRHLQGREDNLTLDISKLKEQIFEASKAHLNLVPGTEAIAGVADGLANLNPVTWVKT
IGSTTIINLILILVCLFCLLLVCRCTQQLRRDSDHRERAMMTMAVLSKRKGGNVGKSKRDQIVTV
SV
Gag sequence from breast cancer patient viral peptide
SEQ ID NO: 46
tatgcctcttatctcagctttattaaaattcttttaaaaagagggggagttaaagtatctacaaa
aaatctaatcaagctatttcaaataatagaacaattttgcccatggtttccagaacaaggaactt
tagatctaaaagattggaaaagaattggtaaggaactaaaacaagcaggtaggaagggtaatatc
attccacttacagtatggaatgattgggccattattaaagcagctttagaaccatttcaaacaga
agaagatagcgtttcagtttctgatgcccctggaagctgtttaatagattgtaatgaaaagacaa
ggaaaaaatcccagaaagaaacggaaagtttacattgcgaatatgtagcagagccggtaatggct
cagtcaacgcaaaatgttgactataatcaattacaggaggtgatatatcctgaaacgttaaaatt
agaaggaaaaggtcccgaattaatggggccatcagagtctaaaccacgaggcacaagtcctcttc
cagcaggtcaggtgcctgtaacattacaacctcaaacgcaggttaaagaaaataagacccaaccg
ccagtagcctatcaatactggccgccggctgaacttcagtatcggccacccccagaaagtcagta
tggatatccaggaatgcccccagcaccacagggcagggcgccataccctcagccgcccactagga
gacttaatcctatggcaccacctagtagacagggtagtgaattacatgaaattattgataaatca
agaaaggaaggagatactgaggcatggcaattcccagtaacgttagaaccgatgccacctggaga
aggagcccaagagggagagcctcccacagttgaggccagatacaagtctttttcgataaaaatgc
taaaagatatgaaagagggagtaaaacagtatggacccaactccccttatatgaggacattatta
gattccattgctcatggacatagactcattccttatgattgggagattctggcaaaatcgtctct
ctcaccctctcaatttttacaatttaagacttggtggattgatggggtacaagaacaggtccgaa
gaaatagggctgccaatcctccagttaacatagatgcagatcaactattaggaataggtcaaaat
tggagtactattagtcaacaagcattaatgcaaaatgaggccattgagcaagttagagctatctg
ccttagagcctgggaaaaaatccaagacccaggaagtacctgcccctcatttaatacagtaagac
aaggttcaaaagagccctatcctgattttgtggcaaggctccaagatgttgctcaaaagtcaatt
gccgatgaaaaagcccgtaaggtcatagtggagttgatggcatatgaaaacgccaatcctgagtg
tcaatcagccattaagccattaaaaggaaaggttcctgcaggatcagatgtaatctcagaatatg
taaaagcctgtgatggaatcggaggagctatgcataaagctatgcttatggctcaagcaataaca
ggagttgttttaggaggacaagttagaacatttggaggaaaatgttataattgtggtcaaattgg
tcacttaaaaaagaattgcccagtcttaaataaacagaatataactattcaagcaactacaacag
gtagagagccacctgacttatgtccaagatgtaaaaaaggaaaacattgggctagtcaatgtcgt
tctaaatttgataaaaatgggcaaccattgtcgggaaacgagcaaaggggccagcctcaggcccc
acaacaaactggggcattcccaattcagccatttgttcct
Gag sequence from breast cancer patient viral peptide
SEQ ID NO: 47
YASYLSFIKILLKRGGVKVSTKNLIKLFQIIEQFCPWFPEQGTLDLKDWKRIGKELKQAGRKGNI
IPLTVWNDWAIIKAALEPFQTEEDSVSVSDAPGSCLIDCNEKTRKKSQKETESLHCEYVAEPVMA
QSTQNVDYNQLQEVIYPETLKLEGKGPELMGPSESKPRGTSPLPAGQVPVTLQPQTQVKENKTQP
PVAYQYWPPAELQYRPPPESQYGYPGMPPAPQGRAPYPQPPTRRLNPMAPPSRQGSELHEIIDKS
RKEGDTEAWQFPVTLEPMPPGEGAQEGEPPTVEARYKSFSIKMLKDMKEGVKQYGPNSPYMRTLL
DSIAHGHRLIPYDWEILAKSSLSPSQFLQFKTWWIDGVQEQVRRNRAANPPVNIDADQLLGIGQN
WSTISQQALMQNEAIEQVRAICLRAWEKIQDPGSTCPSFNTVRQGSKEPYPDFVARLQDVAQKSI
ADEKARKVIVELMAYENANPECQSAIKPLKGKVPAGSDVISEYVKACDGIGGAMHKAMLMAQAIT
GVVLGGQVRTFGGKCYNCGQIGHLKKNCPVLNKQNITIQATTTGREPPDLCPRCKKGKHWRGQPQ
APQQTGAFPIQPFVPASQCRSKFDKNGQPLSGNEQ
Pol sequence from breast cancer patient viral peptide
SEQ ID NO: 48
aataaatcaagaaagagaaggaatagggtatcctttttaggggcggccactgtagagcctcctaa
acccataccattaacttggaaaacagaaaaaccagtgtgggtaaatcagtggccgctaccaaaac
aaaaactggaggctttacatttattagcaaatgaacagttagaaaagggtcatattgagccttca
ttctcaccttggaattctcctgtgtttgtaattcagaagaaatcaggcaaatggcgtatgttaac
tgacttaagggccgtaaacgccgtaattcaacccatggggcctctccaacccgggttgccctctc
cggccatgatcccaaaagattggcctttaattataattgatctaaaggattgcttttttaccatc
cctctggcagagcaggattgtgaaaaatttgcctttactataccagccataaataataaagaacc
agccaccaggtttcagtggaaagtgttacctcagggaatgcttaatagtccaactatttgtcaga
cttttgtaggtcgagctcttcaaccagttagagaaaagttttcagactgttatattattcattat
attgatgatattttatgtgctgcagaaacgaaagataaattaattgactgttatacatttctgca
agcagaggttgccaatgctggactggcaatagcatctgataagatccaaacctctactccttttc
attatttagggatgcagatagaaaatagaaaaattaagccacaaaaaatagaaataagaaaagac
acattaaaaacactaaatgattttcaaaaattgctgggagatattaattggattcggccaactct
aggcattcctacttatgccatgtcaaatttgttctctatcttaagaggagactcagacttaaata
gtaaaagaatgttaaccccagaggcaacaaaagaaattaaattagtggaagaaaaaattcagtca
gcgcaaataaatagaatagatcccttagccccactccaacttttgatttttgccactgcacattc
tccaacaggcatcattattcaaaatactgatcttgtggagtggtcattccttcctcacagtacag
ttaagacttttacattgtacttggatcaaatagctacattaatcggtcagacaagattacgaata
ataaaattatgtggaaatgacccagacaaaatagttgtccctttaaccaaggaacaagttagaca
agcctttatcaattctggtgcatggcagattggtcttgctaattttgtgggaattattgataatc
attacccaaaaacaaagatcttccagttcttaaaattgactacttggattctacctaaaattacc
agacgtgaacctttagaaaatgctctaacagtatttactgatggttccagcaatggaaaagcagc
ttacacagggctgaaagaacgagtaatcaaaactccatatcaatcggctcaaagagcagagttgg
ttgcagtcattacagtgttacaagattttgaccagcctatcaatattatatcagattctgcatat
gtagtacaggctacaagggatgttgagacagctctaattaaatatagcatggatgatcagttaaa
ccagctattcaatttattacaacaaactgtaagaaaaagaaatttcccattttatattactcata
ttcgagcacacactaatttaccagggcctttgactaaagcaaatgaacaagctgacttactggta
tcatctgcactcataaaagcacaagaacttcatgct
Pol sequence from breast cancer patient viral peptide
SEQ ID NO: 20XX
NKSRKRRNRVSFLGAATVEPPKPIPLTWKTEKPVWVNQWPLPKWKLEALHLLANEQLEKGHIEPS
FSPWNSPVFVIQKKSGKWRMLTDLRAVNAVIQPMGPLQPGLPSPAMIPKDWPLIIIDLKDCFFTI
PLAEQDCEKFAFTIPAINNKEPATRFQWKVLPQGMLNSPTICQTFVGRALQPVREKFSDCYIIHY
IDDILCAAETKDKLIDCYTFLQAEVANAGLAIASDKIQTSTPFHYLGMQIENRKIKPQKIEIRKD
TLKTLNDFQKLLGDINWIRPTLGIPTYAMSNLFSILRGDSDLNSKRMLTPEATKEIKLVEEKIQS
AQINRIDPLAPLQLLIFATAHSPTGIIIQNTDLVEWSFLPHSTVKTFTLYLDQIATLIGQTRLRI
IKLCGNDPDKIVVPLTKEQVRQAFINSGAWQIGLANFVGIIDNHYPKTKIFQFLKLTTWILPKIT
RREPLENALTVFTDGSSNGKAAYTGLKERVIKTPYQSAQRAELVAVITVLQDFDQPINIISDSAY
VVQATRDVETALIKYSMDDQLNQLFNLLQQTVRKRNFPFYITHIRAHTNLPGPLTKANEQADLLV
SSALIKAQELHA

What is claimed is:

1. A HERV-K env gene, wherein the gene has been isolated from a viral particle and promotes tumor development and metastasis, in vitro and in vivo.

2. A method for measuring the increased risk of cancer metastasis in early stages of cancer, comprising the step of:
measuring the increase of HERV-K expression in early stages of cancer;
wherein an increase of HERV-K expression in early stages of cancer indicates the increased risk of cancer metastasis.

3. A human tumor mouse (HTM) model selected from the group consisting of MDA-MB-231 (HTM1) or MDA-MB-468 (HTM2).

4. TCR sequences generated from tumor infiltrating lymphocytes (TILs) that recognize HERV-K antigens as peptides bound to the Major Histocompatibility Complex (MHC), resulting in an interaction between the HLA-peptide complex and the CD8 TCR or CD3 TCR.

5. A platform for enabling functional matching T cell receptor (TCR) sequences, wherein the platform comprises a small number of homogenous HERV-K specific T cell (K-T cell) populations each obtained from a single clonally expanded K-T cell.

6. A method for obtaining cells that secrete IFNγ, comprising the step of obtaining HERV-K specific T cells (K-T cells) from tumor infiltrating lymphocytes or peripheral blood mononuclear cells.

7. A dendritic cell that has been transfected with HERV-K surface (SU) envelope (Env) protein for use as a cancer vaccine.

8. A peripheral blood mononuclear cell, wherein the cell has been in vitro stimulated with their autologous dendritic cells pulsed with KSU protein (K-T cells).

9. A combination therapy method of treating cancer, comprising the administration to a subject in need thereof a therapeutically effective amount of:
(a) a checkpoint inhibitor; and
(b) a HERV-K therapy selected from the group consisting of antibodies, T cell receptors (TCRs), vaccines, peptides, shRNAs, and other drugs.

10. A method of producing antibodies from mice immunized with 5 multiple antigen peptides (MAPs) that are generated from HERV-K SU protein produced by cancer patients.

11. The method of claim 9, wherein the cancer is selected from the group consisting of melanoma, chronic lymphocytic leukemia, breast cancer, pancreatic cancer, head and neck cancer, ovarian cancer, cervical cancer, colorectal cancer, testicular cancer, stomach cancer, kidney cancer, endometrial cancer, uterine cancer, bladder cancer, prostate cancer, esophageal cancer, liver cancer, and non-small cell lung cancer.

12. The use of HERV-K as a stem cell marker.

13. A method for the overexpression of HERV-K, comprising the step of: administering cancer cells with agents that induce expression of HERV-K by innate immune response (Poly I:C treatment) or LTR hypomethylation (5-Aza), wherein the administration provokes the cancer cells to increase production of a target that makes the cancer cells more susceptible to targeted therapy to include targeted immunotherapy.

14. A platform to determine the binding kinetics and cell-to-cell interactions of every cell in a microwell slab, comprising:
(a) polydimethyl siloxane (PDMS) arrays of nanowells, and
(b) cultured cells from mammospheres obtained from patient breast tumor tissues.

15. The use of the immunosuppressive domain (ISD) of HERV-K as an immune checkpoint on cancer cells.

16. The method of claim 15, wherein the immune checkpoint inhibitors of HERV-K are selected from the group consisting of monoclonal antibodies and drugs targeting the ISD of HERV-K.

17. A method of regulating chemotherapeutic drug sensitivity, comprising the step of regulating HERV-K activity.

18. A method of blockading reactive oxygen species signaling, comprising the step of inhibiting HERV-K expression.

19. A method of decreasing reactive oxygen species-mediated induction of epithelial-mesenchymal transition (EMT), comprising the step of inhibiting HERV-K expression.

20. The use of the HERV-K env gene to:
(a) promote expression of an oncogene selected from the group consisting of Ras, p-ERK, c-myc, HIF-1alpha, and AMPK beta; or
(b) downregulate expression of a gene selected form the group consisting of caspase 3, caspase 9, p-RB, CIDEA, p-P38, eNOS, and AMPK alpha.

21. The use of HERV-K as an upstream modulator of the Ras/ERK signaling pathway.

22. A method for activating Ras in humans, that does not involve the mutational activation of Ras genes, comprising the step of increasing HERV-K activity.

23. A method for decreasing Ras activity in humans, comprising the step of decreasing HERV-K expression.

24. Sequences of HERV-K Envelope (Env), Gag, and Pol genes and RNAs extracted from viral particles isolated from breast cancer patients.
